(19) United States
(12) Patent Application Publication
(10) Pub. No.: US 2018/0073073 A1
(43) Pub. Date: Mar. 15, 2018

(54) METHODS AND COMPOSITIONS FOR LABELING TARGETS AND HAPLOTYPE PHASING

(71) Applicant: Cellular Research, Inc., Menlo Park, CA (US)

(72) Inventors: Glenn Fu, Menlo Park, CA (US); Stephen P.A. Fodor, Menlo Park, CA (US)

(21) Appl. No.: 15/557,789

(22) PCT Filed: Mar. 16, 2016
(86) PCT No.: PCT/US2016/022712

§ 371 Date: Sep. 12, 2017

Related U.S. Application Data
(60) Provisional application No. 62/135,018, filed on Mar. 18, 2015.

(51) Int. Cl. C12Q 1/68; C12N 15/10

(52) U.S. Cl. CPC C12Q 1/6874; C12N 15/1065

(57) ABSTRACT

Disclosed herein are methods for estimating the copy number of a target chromosome in a sample. In some embodiments, the methods include: partitioning a sample comprising one or more copies of the first target chromosome, into a plurality of partitioned samples; stochastically barcoding the one or more copies of the first target chromosome in the plurality of partitioned samples using a first plurality of stochastic barcodes, wherein each of the first plurality of stochastic barcodes comprises a first chromosome label and a first molecular label; and estimating the copy number of the first target chromosome in the sample using the first chromosome label and the second molecular label. The disclosed methods can be used for haplotype phasing, aneuploidy determination, and DNA sequencing.

### RELATED APPLICATIONS

The present application claims priority under 35 U.S.C. § 119(e) to U.S. Provisional Application No. 62/135,018, filed on Mar. 18, 2015. The content of this related application is herein expressly incorporated by reference in its entirety.

### BACKGROUND

The present disclosure relates generally to the field of molecular biology and more particularly to haplotype phasing and DNA sequencing.

### DESCRIPTION OF THE RELATED ART

Methods and techniques such as in situ hybridization have been developed for estimation of chromosome copy number in a sample and determination of the aneuploidy of cells. Methods and techniques such as computational phasing allow haplotype phase estimation. However, these methods and techniques can be expensive or can have low accuracy.

### SUMMARY

Disclosed herein are methods for estimating copy number of a target chromosome in a sample. In some embodiments, the methods comprise: providing a sample comprising one or more copies of a first target chromosome; partitioning the sample into a plurality of partitioned samples, wherein each of at least 10% of the plurality of partitioned samples comprises one copy of the first target chromosome; stochastically barcoding the one or more copies of the first target chromosome in the plurality of partitioned samples using a first plurality of stochastic barcodes, wherein each of the first plurality of stochastic barcodes comprises a first chromosome label and a first molecular label; and estimating the copy number of the first target chromosome in the sample using the first chromosome label and the first molecular label.

In some embodiments, partitioning the sample comprises adjusting the volume of the sample to alter the concentration of the first target chromosome in the sample. In some embodiments, partitioning the sample comprises adjusting the volume of the sample partitioned into each of the plurality of partitioned samples.

In some embodiments, each of at least 25% of the plurality of partitioned samples comprises one copy of the first target chromosome. In some embodiments, each of at least 10% of the plurality of partitioned samples comprises one chromosome. In some embodiments, each of at least 25% of the plurality of partitioned samples comprises one chromosome.

In some embodiments, partitioning the sample comprises introducing the plurality of partitioned samples into a plurality of wells of a substrate. Each of the plurality of partitioned samples can be introduced to a well of the plurality of wells. Each of the plurality of partitioned samples can be a droplet in an emulsion.

In some embodiments, stochastically barcoding the one or more copies of the first target chromosome in the plurality of partitioned samples comprises hybridizing the first plurality of stochastic barcodes to the one or more copies of the first target chromosome. Stochastically barcoding the one or more copies of the first target chromosome in the plurality of partitioned samples can comprise generating one or more copies of a stochastically barcoded first target chromosome. Stochastically barcoding the one or more copies of the first target chromosome can comprise generating an indexed library of the stochastically barcoded first target chromosome.

In some embodiments, stochastically barcoding the one or more copies of the first target chromosome comprises fragmenting the one or more copies of the first target chromosome to generate fragments of the first target chromosome. The fragments of the first target chromosome can be at least 10 kilo bases, 100 kilo bases, or 1000 kilo bases in length. The stochastically barcoded first target chromosome can comprise stochastically barcoded fragments of the first target chromosome.

In some embodiments, the first plurality of the stochastic barcodes is associated with a solid support. The solid support can be a synthetic particle. The first molecular labels of the first plurality of stochastic barcodes on the solid support can differ by at least one nucleotide. The first chromosome labels of the first plurality of stochastic barcodes on the solid support can be the same. The first chromosome label can be about 5-20 nucleotides long. The molecular label can be about 5-20 nucleotides long. The synthetic particle can be a bead. The bead can a silica gel bead, a controlled pore glass bead, a magnetic bead, a Dynabead, a Sephadex/Sepharose bead, a cellulose bead, a polystyrene bead, or any combination thereof.

In some embodiments, estimating the copy number of the first target chromosome in the sample comprises determining sequences of at least some of the stochastically barcoded fragments of the first target chromosome in the indexed library. Determining the sequences of the at least some of the stochastically barcoded fragments of the first target chromosome in the indexed library can comprise generating sequences with read lengths of 50 or more bases. The one or more copies of the first target chromosome can be inside one or more cells. In some embodiments, the one or more copies of the first target chromosome can be not inside any cell.

In some embodiments, the one or more copies of the first target chromosome comprise chromosomes from fetal cells. In some embodiments, the one or more copies of the first target chromosome comprise chromosomes from cancer cells. The first target chromosome can be a human chromosome.

In some embodiments, the sample comprises one or more copies of a second target chromosome, and wherein each of at least 10% of the plurality of partitioned samples comprises one copy of the second target chromosome, the methods further comprise: stochastically barcoding one or more copies of the second target chromosome in the plurality of partitioned samples using a second plurality of stochastic barcodes, wherein each of the second plurality of stochastic barcodes comprises a second chromosome label and a second molecular label, and wherein the first chromosome labels of the first plurality of stochastic barcodes and the second chromosome labels of the second plurality of stochastic barcodes differ by at least one nucleotide; and estimating the copy number of the second target chromosome in the sample using the second chromosome label and the second molecular label.

In some embodiments, the sample comprises one or more copies of each of n target chromosomes, wherein n is an integer greater than one, and wherein, for each of the n target chromosomes, each of at least 10% of the plurality of partitioned samples comprises one copy of the n^{th} target chromosomes, the methods further comprises: for each of the n target chromosomes in the plurality of partitioned samples, stochastically barcoding the one or more copies of the n^{th} target chromosome using a n^{th} plurality of stochastic barcodes, wherein each of the n^{th} plurality of stochastic barcodes comprises a n^{th} chromosome label and a n^{th} molecular label, and wherein the first chromosome labels of the first plurality of stochastic barcodes and the n^{h} chromosome labels of the n^{th} plurality of stochastic barcodes differ by at least one nucleotide; and estimating the copy number of each of the plurality of n^{th} target chromosomes in the sample using the n^{th} chromosome label and the n^{th} molecular label. The method can be multiplexed.

Disclosed herein are methods for haplotype phasing two or more gene targets on a target chromosome in a sample. In some embodiments, the methods comprise: providing a sample comprising one or more copies of a target chromosome, wherein the target chromosome comprises two or more gene targets; partitioning the sample into a plurality of partitioned samples, wherein each of at least 10% of the plurality of partitioned samples comprises one copy of the target chromosome; stochastically barcoding the one or more copies of the target chromosome in the plurality of partitioned samples using a plurality of stochastic barcodes, wherein each of the plurality of stochastic barcodes comprises a chromosome label and a molecular label; and determining the haplotype phasing of the two or more gene targets on the target chromosome in the sample using the chromosome label and the molecular label.

In some embodiments, partitioning the sample comprises adjusting the volume of the sample to alter the concentration of the target chromosome in the sample. In some embodiments, partitioning the sample comprises adjusting the volume of the sample partitioned into each of the plurality of partitioned samples. Partitioning the sample can comprise introducing the plurality of partitioned samples into a plurality of wells of a substrate. Each of the plurality of partitioned samples can be introduced to a well of the plurality of wells. Each of the plurality of partitioned samples is a droplet in an emulsion.

In some embodiments, stochastically barcoding the one or more copies of the target chromosome comprises fragmenting the one or more copies of the target chromosome to generate fragments of the target chromosome. The fragments of the target chromosome can be at least 10 kilo bases in length.

In some embodiments, stochastically barcoding the one or more copies of the target chromosome in the plurality of partitioned samples can comprise hybridizing the plurality of stochastic barcodes to the fragments of the target chromosome. Stochastically barcoding the one or more copies of the target chromosome in the plurality of partitioned samples can comprise generating stochastically barcoded fragments of the target chromosome. Stochastically barcoding the one or more copies of the target chromosome can comprise generating an indexed library of the stochastically barcoded fragments of the target chromosome.

In some embodiments, the plurality of the stochastic barcodes is associated with a solid support. The solid support can be a synthetic particle.

In some embodiments, determining the haplotype phasing of the two or more gene targets on the target chromosome comprises determining sequences of at least some of the stochastically barcoded fragments in the indexed library. Determining the sequences of the at least some of the stochastically barcoded fragments in the indexed library can comprise determining sequences of the two or more gene targets.

In some embodiments, the methods further comprise: identifying one or more variations of the two or more gene targets in the sequences of the two or more gene targets determined. At least two of the two or more gene targets can be separated from one another on the target chromosome by at least 10 kilo bases, 100 kilo bases, or 1000 kilo bases.

Disclosed herein are methods for determining aneuploidy of one or more cells. In some embodiments, the methods comprise: providing a sample comprising chromosomes from one or more cells; partitioning the sample into a plurality of partitioned samples, wherein each of at least 10% of the plurality of partitioned samples comprises one copy of a first target chromosome; stochastically barcoding the one or more copies of the first target chromosome in the plurality of partitioned samples using a first plurality of stochastic barcodes, wherein each of the first plurality of stochastic barcodes comprises a first chromosome label and a first molecular label; and determining the aneuploidy of the one or more cells in the sample, wherein determining the aneuploidy of the one or more cells in the sample comprises determining the number of a first gene target on the first target chromosome using the first chromosome label and the first molecular label.

In some embodiments, partitioning the sample comprises adjusting the volume of the sample to alter the concentration of the first target chromosome in the sample. In some embodiments, partitioning the sample comprises adjusting the volume of the sample partitioned into each of the plurality of partitioned samples. Partitioning the sample can comprise introducing the plurality of partitioned samples into a plurality of wells of a substrate. Each of the plurality of partitioned samples can be introduced to a well of the plurality of wells. Each of the plurality of partitioned samples can be a droplet in an emulsion.

In some embodiments, stochastically barcoding the one or more copies of the first target chromosome comprises fragmenting the one or more copies of the first target chromosome to generate fragments of the first target chromosome. The fragments of the first target chromosome can be at least 10 kilo bases in length.

Stochastically barcoding the one or more copies of the first target chromosome in the plurality of partitioned samples comprises hybridizing the first plurality of stochastic barcodes to the fragments of the first target chromosome. Stochastically barcoding the one or more copies of the first target chromosome in the plurality of partitioned samples can comprise generating stochastically barcoded fragments of the first target chromosome. Stochastically barcoding the one or more copies of the first target chromosome can comprise generating an indexed library of the stochastically barcoded fragments of first target chromosome.

In some embodiments, the plurality of the stochastic barcodes is associated with a solid support. The solid support can be a synthetic particle.

In some embodiments, the aneuploidy is a trisomy. The trisomy can be an autosomal trisomy.

In some embodiments, the sample comprises one or more copies of a second target chromosome, and wherein each of at least 10% of the plurality of partitioned samples comprises one copy of the second target chromosomes, the methods further comprise: stochastically barcoding the one or more copies of the second target chromosome in the plurality of partitioned samples using a second plurality of stochastic barcodes, wherein each of the second plurality of stochastic barcodes comprises a second chromosome label and a second molecular label, wherein stochastically barcoding the one or more copies of the second target chromosome comprises fragmenting the one or more copies of the second target chromosome to generate fragments of the second target chromosome and generating an indexed library of stochastically barcoded fragments of the second target chromosome, and wherein determining the aneuploidy of the one or more cells in the sample further comprises determining the number of a second gene target on the second target chromosome using the second chromosome label and the second molecular label and comparing the number of the first gene target and the number of the second gene target.

In some embodiments, the sample comprises one or more copies of each of n target chromosomes, wherein n is an integer greater than one, and wherein each of the plurality of partitioned samples comprises one copy of each of the n target chromosomes, the methods further comprise: for each of the n target chromosomes in the plurality of partitioned samples, stochastically barcoding the one or more copies of the n^{th} target chromosome using a n^{th} plurality of stochastic barcodes, wherein each of the n^{th} stochastic barcodes comprises a n^{th} chromosome label and a n^{th} molecular label, wherein stochastically barcoding the one or more copies of the n^{th} target chromosome comprises fragmenting the one or more copies of the n^{th} target chromosome to generate fragments of the n^{th} target chromosome and generating an indexed library of stochastically barcoded fragments of the n^{th} target chromosome, and wherein determining the aneuploidy of the one or more cells in the sample further comprises, for each of n target chromosomes, determining the number of a n^{th} gene target on the n^{th} target chromosome in the indexed library and comparing the number of the first gene target and the number of the n^{th} gene target.

Disclosed herein are methods for sequencing a first target chromosome in a sample. In some embodiments, the methods comprise: providing a sample comprising one or more copies of a first target chromosome; partitioning the sample into a plurality of partitioned samples, wherein each of at least 10% of the plurality of partitioned samples comprises one copy of the first target chromosome; stochastically barcoding the one or more copies of the first target chromosome in the plurality of partitioned samples using a first plurality of stochastic barcodes, wherein each of the first plurality of stochastic barcodes comprises a first chromosome label and a first molecular label; and obtaining sequence information of the first target chromosome using the first chromosome label and the first molecular label.

In some embodiments, partitioning the sample comprises adjusting the volume of the sample to alter the concentration of the first target chromosome in the sample. In some embodiments, partitioning the sample comprises adjusting the volume of the sample partitioned into each of the plurality of partitioned samples. Partitioning the sample can comprise introducing the plurality of partitioned samples into a plurality of wells of a substrate. Each of the plurality of partitioned samples can be introduced to a well of the plurality of wells. Each of the plurality of partitioned samples can be a droplet in an emulsion.

In some embodiments, stochastically barcoding the one or more copies of the first target chromosome comprises fragmenting the one or more copies of the first target chromosome to generate fragments of the first target chromosome. The fragments of the first target chromosome can be at least 10 kilo bases(kb) in length.

In some embodiments, stochastically barcoding the one or more copies of the first target chromosome in the plurality of partitioned samples comprises hybridizing the plurality of stochastic barcodes to the fragments of the first target chromosome. Stochastically barcoding the one or more copies of the first target chromosome in the plurality of partitioned samples can comprise generating stochastically barcoded fragments of the first target chromosome. Stochastically barcoding the one or more copies of the first target chromosome can comprise generating an indexed library of the stochastically barcoded fragments of the first target chromosome.

In some embodiments, the plurality of the stochastic barcodes is associated with a solid support. The solid support can be a synthetic particle.

In some embodiments, obtaining the sequence information of the first target chromosome comprises determining sequences of at least some of the stochastically barcoded fragments in the indexed library. Determining the sequences of the at least some of the stochastically barcoded fragments of the first target chromosome in the indexed library can comprise generating sequences with read lengths of 50 or more bases. Sequencing the at least some of the stochastically barcoded fragments in the indexed library can comprise deconvoluting the sequencing result from sequencing the indexed library. Deconvoluting the sequencing result can comprise using a software-as-a-service platform. In some embodiments, obtaining the sequence information of the first target chromosome comprises obtaining the sequence information of at least 10% of the base pairs of the first target chromosome.

In some embodiments, the sample comprises one or more copies of a second target chromosome, and wherein each of at least 10% of the plurality of partitioned samples comprises one copy of the second target chromosome, the method further comprise: stochastically barcoding the one or more copies of the second target chromosome in the plurality of partitioned samples using a second plurality of stochastic barcodes, wherein each of the second plurality of stochastic barcodes comprises a second chromosome label and a second molecular label, and wherein the first chromosome labels of the first plurality of stochastic barcodes and the second chromosome labels of the second plurality of stochastic barcodes differ by at least one nucleotide, wherein stochastically barcoding the one or more copies of the second target chromosome comprises fragmenting the one or more copies of the second target chromosome to generate fragments of the second target chromosome and generating an indexed library of stochastically barcoded fragments of the second target chromosome; obtaining sequence information of the second target chromosome using the second chromosome label and the second molecular label, wherein obtaining sequence information of the second target chromosome comprises determining sequences of at least some of the stochastically barcoded fragments of the second target chromosome in the indexed library.

In some embodiments, the sample comprises one or more copies of each of n target chromosomes, and wherein, for each of the n target chromosomes, each of at least 10% of the plurality of partitioned samples comprises one copy of the n^{th} target chromosome, the method further comprises: for each of the n target chromosomes, stochastically barcoding the one or more copies of the n^{th} target chromosome in the plurality of partitioned samples using a n^{th} plurality of stochastic barcodes, wherein each of the n^{th} plurality of stochastic barcodes comprises a n^{th} chromosome label and a n^{th} molecular label, and wherein the first chromosome labels of the first plurality of stochastic barcodes and the n^{th} chromosome labels of the n^{th} plurality of stochastic barcodes differ by at least one nucleotide, and wherein stochastically barcoding the one or more copies of the n^{th} target chromosome comprises fragmenting the one or more copies of the n^{th} target chromosome to generate fragments of the n^{th} target chromosome and generating an indexed library of stochastically barcoded fragments of the n^{th} target chromosome; for each of the n target chromosomes, obtaining sequence information of the n^{th} target chromosome using the n^{th} chromosome label and the n^{th} molecular label, wherein obtaining sequence information of the n^{th} target chromosome comprises determining sequences of at least some of the stochastically barcoded fragments of n^{th} target chromosome in the indexed library.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a non-limiting exemplary stochastic barcode.

FIG. 2 shows a non-limiting exemplary workflow of stochastic barcoding and digital counting.

FIG. 3 is a schematic illustration showing a non-limiting exemplary process for generating an indexed library of the stochastically barcoded targets from a plurality of targets.

FIG. 4 is a flowchart showing non-limiting exemplary steps of data analysis.

FIG. 5 shows a non-limiting exemplary instrument used in the methods of the disclosure.

FIG. 6 illustrates a non-limiting exemplary architecture of a computer system that can be used in connection with embodiments of the present disclosure.

FIG. 7 illustrates a non-limiting exemplary architecture showing a network with a plurality of computer systems for use in the methods of the disclosure.

FIG. 8 illustrates a non-limiting exemplary architecture of a multiprocessor computer system using a shared virtual address memory space in accordance with the methods of the disclosure.

FIGS. 9A-C depict a non-limiting exemplary cartridge for use in the methods of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Methods and compositions for labeling nucleic acid molecules for amplification or sequencing have been developed. Stochastic counting on nucleic acid targets is an important quantification method. Stochastic counting can be used to determine genetic phasing. Disclosed herein are methods and compositions for labeling targets for stochastic counting.

A method for estimating the copy number of chromosomes in a sample is disclosed. In some embodiments, the method comprises: contacting the chromosomes to a microwell in a substrate; associating the chromosomes in the sample with a stochastic barcode attached to a solid support; amplifying the chromosomes; and estimating the copy number of the chromosomes by determining a portion of the sequence of the targets. In some embodiments, the contacting comprises diluting the chromosomes. In some embodiments, the chromosomes are inside a cell. In some embodiments, the chromosomes are outside of a cell. In some embodiments, the chromosomes comprise gene fragments originating from the chromosomes. In some embodiments, the sample is from a pregnant woman. In some embodiments, the sample is a fetal sample.

A method for determining haplotype phasing of a target in a sample is disclosed. In some embodiments, the method comprises: contacting the sample to a microwell in a substrate; associating the target in the sample with a stochastic barcode attached to a solid support, amplifying the target; and determining haplotype phasing of the target. In some embodiments, the determining haplotype phasing comprises determining if the target originated from a maternal chromosome. In some embodiments, the determining haplotype phasing comprises determining if the target originated from a paternal chromosome. In some embodiments, the determining haplotype phasing comprises determining the parental origin of the target.

A method for determining aneuploidy of a sample is disclosed. In some embodiments, the method comprises: contacting the sample to a microwell in a substrate; associating one or more targets in the sample with a stochastic barcode attached to a solid support; amplifying the one or more targets; and determining the aneuploidy of the sample. In some embodiments, the determining comprises determining autosomal trisomies. In some embodiments, the sample is from a pregnant woman. In some embodiments, the sample is a fetal sample.

### Definitions

Unless defined otherwise, technical and scientific terms used herein have the same meaning as commonly understood by one of ordinary skill in the art to which the present disclosure belongs. See. e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, the term “adaptor” can mean a sequence to facilitate amplification or sequencing of associated nucleic acids. The associated nucleic acids can comprise target nucleic acids. The associated nucleic acids can comprise one or more of spatial labels, target labels, sample labels, indexing label, barcodes, stochastic barcodes, or molecular labels. The adapters can be linear. The adaptors can be pre-adenylated adapters. The adaptors can be double- or single-stranded. One or more adaptor can be located on the 5′ or 3′ end of a nucleic acid. When the adaptors comprise known sequences on the 5′ and 3′ ends, the known sequences can be the same or different sequences. An adaptor located on the 5′ and/or 3′ ends of a polynucleotide can be capable of hybridizing to one or more oligonucleotides immobilized on a surface. An adapter can, in some embodiments, comprise a universal sequence. A universal sequence can be a region of nucleotide sequence that is common to two or more nucleic acid molecules. The two or more nucleic acid molecules can also have regions of different sequence. Thus, for example, the 5′ adapters can comprise identical and/or universal nucleic acid sequences and the 3′ adapters can comprise identical and/or universal sequences. A universal sequence that may be present in different members of a plurality of nucleic acid molecules can allow the replication or amplification of multiple different sequences using a single universal primer that is complementary to the universal sequence. Similarly, at least one, two (e.g., a pair) or more universal sequences that may be present in different members of a collection of nucleic acid molecules can allow the replication or amplification of multiple different sequences using at least one, two (e.g., a pair) or more single universal primers that are complementary to the universal sequences. Thus, a universal primer includes a sequence that can hybridize to such a universal sequence. The target nucleic acid sequence-bearing molecules may be modified to attach universal adapters (e.g., non-target nucleic acid sequences) to one or both ends of the different target nucleic acid sequences. The one or more universal primers attached to the target nucleic acid can provide sites for hybridization of universal primers. The one or more universal primers attached to the target nucleic acid can be the same or different from each other.

As used herein the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label.

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “digital counting” can refer to a method for estimating a number of target molecules in a sample. Digital counting can include the step of determining a number of unique labels that have been associated with targets in a sample. This stochastic methodology transforms the problem of counting molecules from one of locating and identifying identical molecules to a series of yes/no digital questions regarding detection of a set of predefined labels.

As used herein, the term “label” or “labels” can refer to nucleic acid codes associated with a target within a sample. A label can be, for example, a nucleic acid label. A label can be an entirely or partially amplifiable label. A label can be entirely or partially sequencable label. A label can be a portion of a native nucleic acid that is identifiable as distinct. A label can be a known sequence. A label can comprise a junction of nucleic acid sequences, for example a junction of a native and non-native sequence. As used herein, the term “label” can be used interchangeably with the terms, “index”, “tag,” or “label-tag.” Labels can convey information. For example, in various embodiments, labels can be used to determine an identity of a sample, a source of a sample, an identity of a cell, and/or a target.

As used herein, the term “non-depleting reservoirs” can refer to a pool of stochastic barcodes made up of many different labels. A non-depleting reservoir can comprise large numbers of different stochastic barcodes such that when the non-depleting reservoir is associated with a pool of targets each target is likely to be associated with a unique stochastic barcode. The uniqueness of each labeled target molecule can be determined by the statistics of random choice, and depends on the number of copies of identical target molecules in the collection compared to the diversity of labels. The size of the resulting set of labeled target molecules can be determined by the stochastic nature of the barcoding process, and analysis of the number of stochastic barcodes detected then allows calculation of the number of target molecules present in the original collection or sample. When the ratio of the number of copies of a target molecule present to the number of unique stochastic barcodes is low, the labeled target molecules are highly unique (i.e. there is a very low probability that more than one target molecule will have been labeled with a given label).

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g. altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein. “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,′:4,5)pyrrolo[2,3-d]pyrimidin-2-one).

As used herein, the term “sample” can refer to a composition comprising targets. Suitable samples for analysis by the disclosed methods, devices, and systems include cells, tissues, organs, or organisms.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which a plurality of stochastic barcodes may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A plurality of solid supports spaced in an array may not comprise a substrate. A solid support may be used interchangeably with the term “bead.”

A solid support can refer to a “substrate.” A substrate can be a type of solid support. A substrate can refer to a continuous solid or semi-solid surface on which the methods of the disclosure may be performed. A substrate can refer to an array, a cartridge, a chip, a device, and a slide, for example.

As used here, the term, “spatial label” can refer to a label which can be associated with a position in space.

As used herein, the term “stochastic barcode” can refer to a polynucleotide sequence comprising labels. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “gene-specific stochastic barcode” can refer to a polynucleotide sequence comprising labels and a target-binding region that is gene-specific. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “stochastic barcoding” can refer to the random labeling (e.g., barcoding) of nucleic acids. Stochastic barcoding can utilize a recursive Poisson strategy to associate and quantify labels associated with targets. As used herein, the term “stochastic barcoding” can be used interchangeably with “gene-specific stochastic barcoding.”

As used here, the term “target” can refer to a composition which can be associated with a stochastic barcode. Exemplary suitable targets for analysis by the disclosed methods, devices, and systems include oligonucleotides, DNA, RNA, mRNA, microRNA, tRNA, and the like. Targets can be single or double stranded. In some embodiments targets can be proteins. In some embodiments targets are lipids.

As used herein, the term “reverse transcriptases” can refer to a group of enzymes having reverse transcriptase activity (i.e., that catalyze synthesis of DNA from an RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptascs include non-LTR retrotransposon reverse transcriptases, retroplasmid reverse transcriptases, retron reverse transciptases, and group II intron reverse transcriptases. Examples of group II intron reverse transcriptases include the Lactococcus lactis LI.LtrB intron reverse transcriptase, the Thermosynechococcus elongates TeI4c intron reverse transcriptase, or the Geobacillus stearothermophilus GsI-IIC intron reverse transcriptase. Other classes of reverse transcriptases can include many classes of non-retroviral reverse transcriptases (i.e., retrons, group II introns, and diversity-generating retroelements among others).

The terms “universal adaptor primer,” “universal primer adaptor” or “universal adaptor sequence” are used interchangeably to refer to a nucleotide sequence that can be used to hybridize stochastic barcodes to generate gene-specific stochastic barcodes. A universal adaptor sequence can, for example, be a known sequence that is universal across all stochastic barcodes used in methods of the disclosure. For example, when multiple targets are being labeled using the methods disclosed herein, each of the target-specific sequences may be linked to the same universal adaptor sequence. In some embodiments, more than one universal adaptor sequences may be used in the methods disclosed herein. For example, when multiple targets are being labeled using the methods disclosed herein, at least two of the target-specific sequences are linked to different universal adaptor sequences. A universal adaptor primer and its complement may be included in two oligonucleotides, one of which comprises a target-specific sequence and the other comprises a stochastic barcode. For example, a universal adaptor sequence may be part of an oligonucleotide comprising a target-specific sequence to generate a nucleotide sequence that is complementary to a target nucleic acid. A second oligonucleotide comprising a stochastic barcode and a complementary sequence of the universal adaptor sequence may hybridize with the nucleotide sequence and generate a target-specific stochastic barcode. In some embodiments, a universal adaptor primer has a sequence that is different from a universal PCR primer used in the methods of this disclosure.

### Stochastic Barcodes

Stochastic barcoding has been described in, for example, US20150299785 and WO2015031691, the content of these applications is incorporated hereby in its entirety.

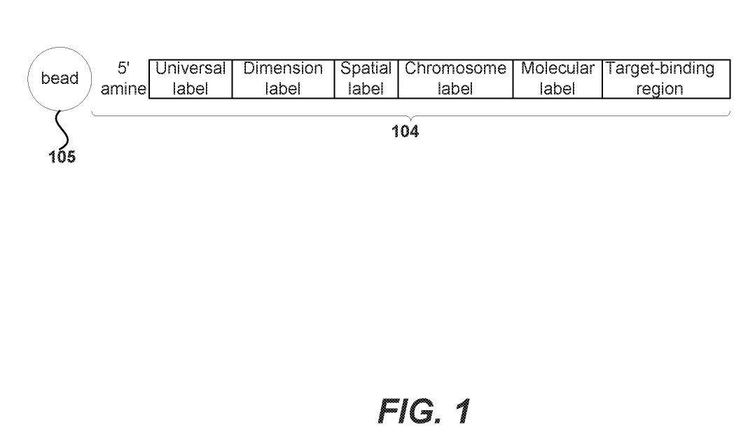

A stochastic barcode is a polynucleotide sequence that may be used to stochastically label (e.g., barcode, tag) a target. A stochastic barcode can comprise one or more labels. Exemplary labels can include a universal label, a chromosome label, a molecular label, a sample label, a plate label, a spatial label, and/or a pre-spatial label. FIG. 1 illustrates an exemplary stochastic barcode 104 with a spatial label. The stochastic barcode 104 can comprise a 5′amine that may link the stochastic barcode to a solid support 105. The stochastic barcode can comprise a universal label, a dimension label, a spatial label, a chromosome label, and/or a molecular label. The order of different labels (including but not limited to the universal label, the dimension label, the spatial label, the chromosome label, and the molecule label) in the stochastic barcode can vary. For example, as shown in FIG. 1, the universal label may be the 5′-most label, and the molecular label may be the 3′-most label. The spatial label, dimension label, and the chromosome label may be in any order. In some embodiments, the universal label, the spatial label, the dimension label, the chromosome label, and the molecular label are in any order.

A label, for example the chromosome label, can comprise a unique set of nucleic acid sub-sequences of defined length, e.g. 7 nucleotides each (equivalent to the number of bits used in some Hamming error correction codes), which can be designed to provide error correction capability. The set of error correction sub-sequences comprise 7 nucleotide sequences can be designed such that any pairwise combination of sequences in the set exhibits a defined “genetic distance” (or number of mismatched bases), for example, a set of error correction sub-sequences can be designed to exhibit a genetic distance of 3 nucleotides. In this case, review of the error correction sequences in the set of sequence data for labeled target nucleic acid molecules (described more fully below) can allow one to detect or correct amplification or sequencing errors. In some embodiments, the length of the nucleic acid sub-sequences used for creating error correction codes can vary, for example, they can be 3 nucleotides, 7 nucleotides, 15 nucleotides, or 31 nucleotides in length. In some embodiments, nucleic acid sub-sequences of other lengths can be used for creating error correction codes.

The stochastic barcode can comprise a target-binding region. The target-binding region can interact with a target in a sample. The target can be, or comprise, ribonucleic acids (RNAs), messenger RNAs (mRNAs), microRNAs, small interfering RNAs (siRNAs), RNA degradation products, RNAs each comprising a poly(A) tail, and any combination thereof. In some embodiments, the plurality of targets can include deoxyribonucleic acids (DNAs).

In some embodiments, a target-binding region can comprise an oligo(dT) sequence which can interact with poly(A) tails of mRNAs. One or more of the labels of the stochastic barcode (e.g., the universal label, the dimension label, the spatial label, the chromosome label, and the molecular label) can be separated by a spacer from another one or two of the remaining labels of the stochastic barcode. The spacer can be, for example, 1, 2, 3, 4, 5, 6, 7, 8, 9, 10, 11, 12, 13, 14, 15, 16, 17, 18, 19, or 20 or more nucleotides. In some embodiments, none of the labels of the stochastic barcode is separated by spacer.

### Universal Labels

A stochastic barcode can comprise one or more universal labels. In some embodiments, the one or more universal labels can be the same for all stochastic barcodes in the set of stochastic barcodes attached to a given solid support. In some embodiments, the one or more universal labels can be the same for all stochastic barcodes attached to a plurality of beads. In some embodiments, a universal label can comprise a nucleic acid sequence that is capable of hybridizing to a sequencing primer. Sequencing primers can be used for sequencing stochastic barcodes comprising a universal label. Sequencing primers (e.g., universal sequencing primers) can comprise sequencing primers associated with high-throughput sequencing platforms. In some embodiments, a universal label can comprise a nucleic acid sequence that is capable of hybridizing to a PCR primer. In some embodiments, the universal label can comprise a nucleic acid sequence that is capable of hybridizing to a sequencing primer and a PCR primer. The nucleic acid sequence of the universal label that is capable of hybridizing to a sequencing or PCR primer can be referred to as a primer binding site. A universal label can comprise a sequence that can be used to initiate transcription of the stochastic barcode. A universal label can comprise a sequence that can be used for extension of the stochastic barcode or a region within the stochastic barcode. A universal label can be, can be about, can be at least, or can be at least about, 1, 2, 3, 4, 5, 10, 15, 20, 25, 30, 35, 40, 45, 50, or a number or a range between any two of these values, nucleotides in length. A universal label can comprise at least about 10 nucleotides. A universal label can be, can be at most, or can be at most about, 1, 2, 3, 4, 5, 10, 15, 20, 25, 30, 35, 40, 45, 50, or a number or a range between any two of these values, nucleotides in length. In some embodiments, a cleavable linker or modified nucleotide can be part of the universal label sequence to enable the stochastic barcode to be cleaved off from the support.

### Dimension Labels

A stochastic barcode can comprise one or more dimension labels. In some embodiments, a dimension label can comprise a nucleic acid sequence that provides information about a dimension in which the stochastic labeling occurred. For example, a dimension label can provide information about the time at which a target was stochastically barcoded. A dimension label can be associated with a time of stochastic barcoding in a sample. A dimension label can be activated at the time of stochastic labeling. Different dimension labels can be activated at different times. The dimension label provides information about the order in which targets, groups of targets, and/or samples were stochastically barcoded. For example, a population of cells can be stochastically barcoded at the G0 phase of the cell cycle. The cells can be pulsed again with stochastic barcodes at the G1 phase of the cell cycle. The cells can be pulsed again with stochastic barcodes at the S phase of the cell cycle, and so on. Stochastic barcodes at each pulse (e.g., each phase of the cell cycle), can comprise different dimension labels. In this way, the dimension label provides information about which targets were labelled at which phase of the cell cycle. Dimension labels can interrogate many different biological times. Exemplary biological times can include, but are not limited to, the cell cycle, transcription (e.g., transcription initiation), and transcript degradation. In another example, a sample (e.g., a cell, a population of cells) can be stochastically labeled before and/or after treatment with a drug and/or therapy. The changes in the number of copies of distinct targets can be indicative of the sample's response to the drug and/or therapy.

A dimension label can be activatable. An activatable dimension label can be activated at a specific time point. The activatable label can be, for example, constitutively activated (e.g., not turned off). The activatable dimension label can be, for example, reversibly activated (e.g., the activatable dimension label can be turned on and turned off). The dimension label can be, for example, reversibly activatable at least 1, 2, 3, 4, 5, 6, 7, 8, 9, or 10 or more times. The dimension label can be reversibly activatable, for example, at least 1, 2, 3, 4, 5, 6, 7, 8, 9, or 10 or more times. In some embodiments, the dimension label can be activated with fluorescence, light, a chemical event (e.g., cleavage, ligation of another molecule, addition of modifications (e.g., pegylated, sumoylated, acetylated, methylated, deacetylated, demethylated), a photochemical event (e.g., photocaging), and introduction of a non-natural nucleotide.

The dimension label can, in some embodiments, be identical for all stochastic barcodes attached to a given solid support (e.g., bead), but different for different solid supports (e.g., beads). In some embodiments, at least 60%, 70%, 80%, 85%, 90%, 95%, 97%, 99% or 100% of stochastic barcodes on the same solid support can comprise the same dimension label. In some embodiments, at least 60% of stochastic barcodes on the same solid support can comprise the same dimension label. In some embodiments, at least 95% of stochastic barcodes on the same solid support can comprise the same dimension label.

There can be as many as 10⁶ or more unique dimension label sequences represented in a plurality of solid supports (e.g., beads). A dimension label can be, can be about, can be at least, or can be at least about 1, 2, 3, 4, 5, 10, 15, 20, 25, 30, 35, 40, 45, 50, or a number or a range between any two of these values, nucleotides in length. A dimension label can be, can be at most, or can be at most about, 300, 200, 100, 90, 80, 70, 60, 50, 40, 30, 20, 15, 12, 10, 9, 8, 7, 6, 5, 4, 3, 2, 1, or a number or a range between any two of these values, nucleotides in length. A dimension label can comprise between about 5 to about 200 nucleotides. A dimension label can comprise between about 10 to about 150 nucleotides. A dimension label can comprise between about 20 to about 125 nucleotides in length.

### Spatial Labels

A stochastic barcode can comprise one or more spatial labels. In some embodiments, a spatial label can comprise a nucleic acid sequence that provides information about the spatial orientation of a target molecule which is associated with the stochastic barcode. A spatial label can be associated with a coordinate in a sample. The coordinate can be a fixed coordinate. For example a coordinate can be fixed in reference to a substrate. A spatial label can be in reference to a two or three-dimensional grid. A coordinate can be fixed in reference to a landmark. The landmark can be identifiable in space. A landmark can be a structure which can be imaged. A landmark can be a biological structure, for example an anatomical landmark. A landmark can be a cellular landmark, for instance an organelle. A landmark can be a non-natural landmark such as a structure with an identifiable identifier such as a color code, bar code, magnetic property, fluorescents, radioactivity, or a unique size or shape. A spatial label can be associated with a physical partition (e.g. a well, a container, or a droplet). In some embodiments, multiple spatial labels are used together to encode one or more positions in space.

The spatial label can be identical for all stochastic barcodes attached to a given solid support (e.g., bead), but different for different solid supports (e.g., beads). In some embodiments, at least or at least about, 60%, 70%, 80%, 85%, 90%, 95%, 97%, 99%, 100%, or a number or a range between any two of these values, of stochastic barcodes on the same solid support can comprise the same spatial label. In some embodiments, at least 60% of stochastic barcodes on the same solid support can comprise the same spatial label. In some embodiments, at least 95% of stochastic barcodes on the same solid support can comprise the same spatial label.

There can be as many as 10⁶ or more unique spatial label sequences represented in a plurality of solid supports (e.g., beads). A spatial label can be, can be about, can be at least, or can be at least about, 1, 2, 3, 4, 5, 10, 15, 20, 25, 30, 35, 40, 45, 50, or a number or a range between any two of these values, nucleotides in length. A spatial label can be, can be at most, or can be at most about, 300, 200, 100, 90, 80, 70, 60, 50, 40, 30, 20, 15, 12, 10, 9, 8, 7, 6, 5, 4, 3, 2, 1, or a number or a range between any two of these values, nucleotides in length. A spatial label can comprise between about 5 to about 200 nucleotides. A spatial label can comprise between about 10 to about 150 nucleotides. A spatial label can comprise between about 20 to about 125 nucleotides in length.

### Chromosome Labels

A stochastic barcodes can comprise one or more chromosome labels. In some embodiments, a chromosome label can comprise a nucleic acid sequence that provides information for determining which target nucleic acid originated from which chromosome. For example, for labeling human chromosomes, a chromosome label can be used to determine whether a target nucleic acid is from, for example, chromosome 21, or chromosome 18. In some embodiments, the chromosome label is identical for all stochastic barcodes attached to a given solid support (e.g., bead), but different for different solid supports (e.g., beads). In some embodiments, about, at least, or at least about, 60%, 70%, 80%, 85%, 90%, 95%, 97%, 99%, 100%, or a number or a range between any two of these values, of stochastic barcodes on the same solid support can comprise the same chromosome label. In some embodiments, at least 60% of stochastic barcodes on the same solid support can comprise the same chromosome label. In some embodiment, at least 95% of stochastic barcodes on the same solid support can comprise the same chromosome label.

There can be as many as 10⁶ or more unique chromosome label sequences represented in a plurality of solid supports (e.g., beads). A chromosome label can be, can be about, can be at least, or can be at least about, 1, 2, 3, 4, 5, 10, 15, 20, 25, 30, 35, 40, 45, 50, 60, 70, 80, 90, 100, 200, 300, or a number or a range between any two of these values, nucleotides in length. A chromosome label can be, can be at most, or can be at most about, 300, 200, 100, 90, 80, 70, 60, 50, 40, 30, 20, 15, 12, 10, 9, 8, 7, 6, 5, 4, or a number or a range between any two of these values, nucleotides in length. A chromosome label can comprise between about 5 to about 200 nucleotides. A chromosome label can comprise between about 10 to about 150 nucleotides. A chromosome label can comprise between about 20 to about 125 nucleotides in length.

### Molecular Labels

A stochastic barcodes can comprise one or more molecular labels. In some embodiments, a molecular label can comprise a nucleic acid sequence that provides identifying information for the specific type of target nucleic acid species hybridized to the stochastic barcode. A molecular label can comprise a nucleic acid sequence that provides a counter for the specific occurrence of the target nucleic acid species hybridized to the stochastic barcode (e.g., target-binding region). In some embodiments, a diverse set of molecular labels are attached to a given solid support (e.g., bead). In some embodiments, there can be as many as 10′ or more unique molecular label sequences attached to a given solid support (e.g., bead). In some embodiments, there can be as many as 10′ or more unique molecular label sequences attached to a given solid support (e.g., bead). In some embodiments, there can be as many as 10′ or more unique molecular label sequences attached to a given solid support (e.g., bead). In some embodiments, there can be as many as 10² or more unique molecular label sequences attached to a given solid support (e.g., bead). A molecular label can be at least about 1, 2, 3, 4, 5, 10, 15, 20, 25, 30, 35, 40, 45, 50 or more nucleotides in length. A molecular label can be at most about 300, 200, 100, 90, 80, 70, 60, 50, 40, 30, 20, 15, 12, 10, 9, 8, 7, 6, 5, 4 or fewer nucleotides in length.

### Target-Binding Region

A stochastic barcodes can comprise one or more target binding regions. In some embodiments, a target-binding region can hybridize with a target of interest. In some embodiments, the target binding regions can comprise a nucleic acid sequence that hybridizes specifically to a target (e.g. target nucleic acid, target molecule, e.g., a cellular nucleic acid to be analyzed), for example to a specific gene sequence. In some embodiments, a target binding region can comprise a nucleic acid sequence that can attach (e.g., hybridize) to a specific location of a specific target nucleic acid. In some embodiments, the target binding region can comprise a nucleic acid sequence that is capable of specific hybridization to a restriction enzyme site overhang (e.g. an EcoRI sticky-end overhang). The stochastic barcode can then ligate to any nucleic acid molecule comprising a sequence complementary to the restriction site overhang.

In some embodiments, a target binding region can comprise a non-specific target nucleic acid sequence. A non-specific target nucleic acid sequence can refer to a sequence that can bind to multiple target nucleic acids, independent of the specific sequence of the target nucleic acid. For example, target binding region can comprise a random multimer sequence, or an oligo(dT) sequence that hybridizes to the poly(A) tail on mRNA molecules. A random multimer sequence can be, for example, a random dimer, trimer, quatramer, pentamer, hexamer, septamer, octamer, nonamer, decamer, or higher multimer sequence of any length. In some embodiments, the target binding region is the same for all stochastic barcodes attached to a given bead. In some embodiments, the target binding regions for the plurality of stochastic barcodes attached to a given bead can comprise two or more different target binding sequences. A target binding region can be at least about 5, 10, 15, 20, 25, 30, 35, 40, 45, 50 or more nucleotides in length. A target binding region can be at most about 5, 10, 15, 20, 25, 30, 35, 40, 45, 50 or more nucleotides in length.

In some embodiments, a target-binding region can comprise an oligo(dT) which can hybridize with mRNAs comprising poly-adenylated ends. A target-binding region can be gene-specific. For example, a target-binding region can be configured to hybridize to a specific region of a target. A target-binding region can be, can be about, can be at least, or can be at least about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these values, nucleotides in length. A target-binding region can be, can be at most, or can be at most about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these values, nucleotides in length. A target-binding region can be about 5-30 nucleotides in length. When a stochastic barcode comprises a gene-specific target-binding region, the stochastic barcode can be referred to as a gene-specific stochastic barcode.

### Orientation Property

A stochastic barcode can comprise one or more orientation properties which can be used to orient (e.g., align) the stochastic barcodes. A stochastic barcode can comprise a moiety for isoelectric focusing. Different stochastic barcodes can comprise different isoelectric focusing points. When these stochastic barcodes are introduced to a sample, the sample can undergo isoelectric focusing in order to orient the stochastic barcodes into a known way. In this way, the orientation property can be used to develop a known map of stochastic barcodes in a sample. Exemplary orientation properties can include, electrophoretic mobility (e.g., based on size of the stochastic barcode), isoelectric point, spin, conductivity, and/or self-assembly. For example, stochastic barcodes with an orientation property of self-assembly, can self-assemble into a specific orientation (e.g., nucleic acid nanostructure) upon activation.

### Affinity Property

A stochastic barcode can comprise one or more affinity properties. For example, a spatial label can comprise an affinity property. An affinity property can include a chemical and/or biological moiety that can facilitate binding of the stochastic barcode to another entity (e.g., cell receptor). For example, an affinity property can comprise an antibody, for example, an antibody specific for a specific moiety (e.g., receptor) on a sample. In some embodiments, the antibody can guide the stochastic barcode to a specific cell type or molecule. Targets at and/or near the specific cell type or molecule can be stochastically labeled. The affinity property can, in some embodiments, provide spatial information in addition to the nucleotide sequence of the spatial label because the antibody can guide the stochastic barcode to a specific location. The antibody can be a therapeutic antibody, for example a monoclonal antibody or a polyclonal antibody. The antibody can be humanized or chimeric. The antibody can be a naked antibody or a fusion antibody.

The antibody can be a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

The antibody fragment can be, for example, a portion of an antibody such as F(ab′)2, Fab′, Fab, Fv, sFv and the like. In some embodiments, the antibody fragment can bind with the same antigen that is recognized by the full-length antibody. The antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (CD8, CD34, CD45), and therapeutic antibodies.

### Universal Adaptor Primer

A stochastic barcode can comprise one or more universal adaptor primers. For example, a gene-specific stochastic barcode can comprise a universal adaptor primer. A universal adaptor primer can refer to a nucleotide sequence that is universal across all stochastic barcodes. A universal adaptor primer can be used for building gene-specific stochastic barcodes. A universal adaptor primer can be at least 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 or more nucleotides in length. A universal adaptor primer can be at most 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 or more nucleotides in length. A universal adaptor primer can be from 5-30 nucleotides in length.

### Estimating Copy Number of a Target Chromosome in a Sample

Disclosed herein are methods, compositions, devices, systems and systems for estimating copy number of a target chromosome in a sample. In some embodiments, the methods comprise: providing a sample comprising one or more copies of a first target chromosome; partitioning the sample into a plurality of partitioned samples, wherein each of a desirable percentage of the plurality of partitioned samples comprises one copy of the first target chromosome; stochastically barcoding the one or more copies of the first target chromosome in the plurality of partitioned samples using a first plurality of stochastic barcodes, wherein each of the first plurality of stochastic barcodes comprises a first chromosome label and a first molecular label; and estimating the copy number of the first target chromosome in the sample using the first chromosome label and the first molecular label. In some embodiments, the first chromosome label can be used to identify the first target chromosome. The desirable percentage of the plurality of partitioned sample can be, can be about, can be at least, or can be at most, for example, 10%, 15%, 20%, 25%, 30%, 40%, 50%, 60%, 70%, 80%, 90%, 95%, 99%, or a number or a range between any two of these values, of the plurality of partitioned sample.

In some embodiments, estimating the copy number of the first target chromosome in the sample comprises determining sequences of at least some of the stochastically barcoded fragments of the first target chromosome in the indexed library. The number of stochastically barcoded fragments of the first target chromosome in the indexed library with sequences determined can vary. In some embodiments, the number of stochastically barcoded fragments with sequences determined can be, can be about, can be more than, can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, 10⁴, 10⁵, 10⁶, 10⁷, 10⁸, 10⁹, 10¹⁰, or a number or a range between any two of these values.

Determining the sequences of the at least some of the stochastically barcoded fragments of the first target chromosome in the indexed library can comprise generating sequences. Read lengths of the sequences generated can vary. In some embodiments, read lengths can be, can be about, can be at least, or can be at most, 10, 20, 30, 40, 50, 60, 70, 80, 90, 100, 200, 300, 400, 500, 600, 700, 800, 900, 1000, 10⁴, 10⁵, 10⁶, 10⁷, 10⁸, 10⁹, 10¹⁰, or a number or a range between any two of these values, bases.

In some embodiments, the sample can comprise more than one target chromosomes. For example, the sample can comprise one or more copies of a first target chromosome and one or more copies of a second target chromosome. Each of at least 10% of the plurality of partitioned samples can comprise one copy of the first target chromosome. Each of at least 10% of the plurality of partitioned samples can comprise one copy of the second target chromosome. In some embodiments, the methods further comprise: stochastically barcoding one or more copies of the first target chromosome in the plurality of partitioned samples using a first plurality of stochastic barcodes, and stochastically barcoding one or more copies of the second target chromosome in the plurality of partitioned samples using a second plurality of stochastic barcodes.

Each of the first plurality of stochastic barcodes can comprise a first chromosome label and a first molecular label. Each of the second plurality of stochastic barcodes can comprise a second chromosome label and a second molecular label. The first chromosome label can be used to identify the first target chromosome. The second chromosome label can be used to identify the second target chromosome. The first chromosome labels can be the same. The second chromosome labels can be the same. The first chromosome labels of the first plurality of stochastic barcodes and the second chromosome labels of the second plurality of stochastic barcodes can be different. In some embodiments, the first chromosome labels of the first plurality of stochastic barcodes and the second chromosome labels of the second plurality of stochastic barcodes differ by, by about, by at least, or by at most, 1, 2, 3, 4, 5, 6, 7, 8, 9, 10, 20, 30, 40, 50, 60, 70, 80, 90, 100, 200, 300, or a number or a range between any two of these values, nucleotides. In some embodiments, the methods further comprise: estimating the copy number of the second target chromosome in the sample using the second chromosome label and the second molecular label.

In some embodiments, the sample can comprise a plurality of target chromosomes. For example, the sample can comprise one or more copies of a first target chromosome and one or more copies of each of n target chromosomes, wherein n is an integer greater than one. Each of at least 10% of the plurality of partitioned samples can comprise one copy of the first target chromosome. For each of the n target chromosomes, each of at least 10% of the plurality of partitioned samples can comprise one copy of the n^{th} target chromosomes. In some embodiments, the methods further comprises: for each of the n target chromosomes in the plurality of partitioned samples, stochastically barcoding the one or more copies of the n^{th} target chromosome using a n^{th} plurality of stochastic barcodes.

Each of the first plurality of stochastic barcodes can comprise a first chromosome label and a first molecular label. Each of the n^{th} plurality of stochastic barcodes can comprise a n^{th} chromosome label and a n^{th} molecular label. The first chromosome label can be used to identify the first target chromosome. The n^{th} chromosome label can be used to identify the n^{th} target chromosome. The first chromosome labels of the first plurality of stochastic barcodes can be the same. The n^{th} chromosome labels of the n^{th} plurality of stochastic barcodes can be the same. The first chromosome labels of the first plurality of stochastic barcodes and the n^{th} chromosome labels of the n^{th} plurality of stochastic barcodes can be different. In some embodiments, the first chromosome labels of the first plurality of stochastic barcodes and the n^{th} chromosome labels of the n^{th} plurality of stochastic barcodes can differ by, by about, by at least, or by at most, 1, 2, 3, 4, 5, 6, 7, 8, 9, 10, 20, 30, 40, 50, 60, 70, 80, 90, 100, 200, 300, or a number or a range between any two of these values, nucleotides. Chromosome labels of two different pluralities of stochastic barcodes, for example a first chromosome labels of a first plurality of stochastic barcodes and a second chromosome labels of a second plurality of stochastic barcodes, can be different. In some embodiments, chromosome labels of two different pluralities of stochastic barcodes can differ by, by about, by at least, or by at most, 1, 2, 3, 4, 5, 6, 7, 8, 9, 10, 20, 30, 40, 50, 60, 70, 80, 90, 100, 200, 300, or a number or a range between any two of these values, nucleotides.

In some embodiments, the methods further comprise: estimating the copy number of each of the plurality of n^{th} target chromosomes in the sample using the n^{th} chromosome label and the n^{th} molecular label. In some embodiments, the methods can be multiplexed.

### Sample Partitioning

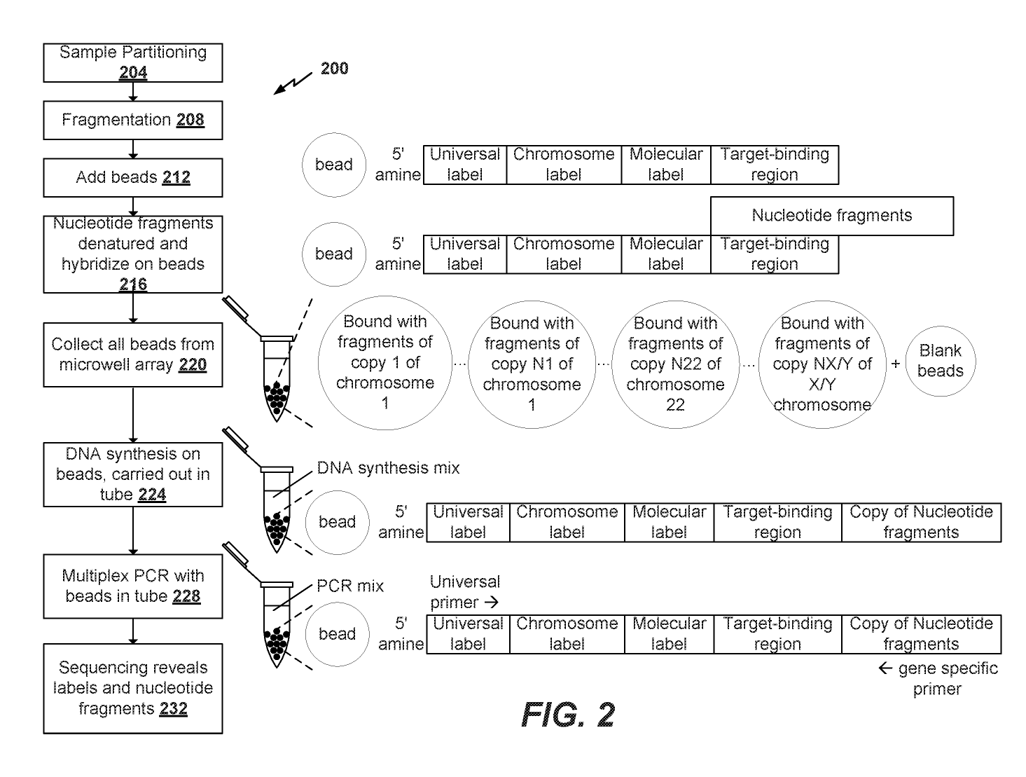

In some embodiments described herein, a sample comprising one or more target chromosomes can be partitioned. For example, in the non-limiting exemplary embodiment of a stochastic barcoding method 200 shown in FIG. 2, at 204, the sample can be partitioned into a plurality of partitioned samples. The plurality of partitioned samples can be, for example, introduced into a plurality of microwells of a well array.

In some embodiments, a sample can be partitioned. Partitioning the sample can comprise introducing the plurality of partitioned samples into a plurality of wells of a substrate. The substrate can be, for example, a well array. In some embodiments, each of the plurality of partitioned samples is introduced to a well of the plurality of wells. In some embodiments, one or more of the plurality of partitioned samples can be a droplet in an emulsion.

In some embodiments, there is one target chromosome (e.g., human chromosome 18). The target chromosome can be, for example, the first target chromosome. In some embodiments, partitioning the sample can comprise adjusting the volume of the sample to alter the concentration of a target chromosome (e.g., the first target chromosome) in the sample. The desired concentration of the target chromosome can vary. In some embodiments, the desired concentration of the target chromosome in the sample can be, can be about, can be more than, or can be at most, one copy of the target chromosome per 1, 2, 3, 4, 5, 6, 7, 8, 9, 10, 20, 30, 40, 50, 60, 70, 80, 90, 100, 200, 300, 400, 500, 600, 700, 800, 900, 1000 microliters, or a number or a range between any two of these values. In some embodiments, the desired concentration of the target chromosome in the sample can be, can be about, can be more than, or can be at most one copy of the target chromosome per 1, 2, 3, 4, 5, 6, 7, 8, 9, 10, 20, 30, 40, 50, 60, 70, 80, 90, 100, 200, 300, 400, 500, 600, 700, 800, 900, 1000 nanoliters, or a number or a range between any two of these values. In some embodiments, the desired concentration of the target chromosome in the sample can be, can be about, can be more than, or can be at most one copy of the target chromosome per 1, 2, 3, 4, 5, 6, 7, 8, 9, 10, 20, 30, 40, 50, 60, 70, 80, 90, 100, 200, 300, 400, 500, 600, 700, 800, 900, 1000 picoliters, or a number or a range between any two of these values. In some embodiments, the desired concentration of the target chromosome in the sample can be, can be about, can be more than, or can be at most one copy of the chromosome per 1, 2, 3, 4, 5, 6, 7, 8, 9, 10, 20, 30, 40, 50, 60, 70, 80, 90, 100, 200, 300, 400, 500, 600, 700, 800, 900, 1000 femtoliters, or a number or a range between any two of these values.

In some embodiments, each of, of about, of more than, or of at least, 1%, 2%, 30, 4%, 5%, 6%, 7%, 8%, 9%, 10%, 20%, 30%, 40%, 50%, 60%, 70%, 80%, 90%, 99.9%, or a number or a range between any two of these values, of the plurality of partitioned samples can comprise one copy of the target chromosome. In some embodiments, each of at least 10% of the plurality of partitioned samples can comprise one copy of the target chromosome. For example, for partitioned samples of 10 picoliters, the desired concentration of the samples can be one copy of the target chromosome per 100 picoliters to achieve that each of at least 10% of the plurality of partitioned sample comprises one copy of the target chromosome. In some embodiments, the sample volume is adjusted to achieve the desired concentration of the target chromosome. In some embodiments, each of at least 20% of the plurality of partitioned samples can comprise one copy of the target chromosome. For example, for partitioned samples of 10 picoliters, the desired concentration of the sample can be two copies of the target chromosome per 100 picoliters to achieve that each of at least 20% of the plurality of partitioned sample comprises one copy of the target chromosome. In some embodiments, the sample volume can be adjusted to achieve the desired concentration of the target chromosome. In some embodiments, each of at least 30% of the plurality of partitioned samples can comprise one copy of the target chromosome. For example, for partitioned samples of 10 picoliters, the desired concentration of the sample can be three copies of the target chromosome per 100 picoliters to achieve that each of at least 30% of the plurality of partitioned sample comprises one copy of the target chromosome. In some embodiments, the sample volume can be adjusted to achieve the desired concentration of the target chromosome.

In some embodiments, partitioning the sample comprises adjusting the volume of the sample partitioned into each of the plurality of partitioned samples. The desired volume of the sample partitioned into each of the plurality of partitioned samples can vary. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 microliters, or a number or a range between any two of these values. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 nanoliters, or a number or a range between any two of these values. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 picoliters, or a number or a range between any two of these values. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 femtoliters, or a number or a range between any two of these values.

In some embodiments, each of at least 10% of the plurality of partitioned samples can comprise one copy of the target chromosome. For example, for a sample with a target chromosome concentration of one copy of the target chromosome per 100 picoliters, the methods can comprise partitioning 10 picoliters of the sample into the plurality of partitioned samples to achieve that each of at least 10% of the plurality of partitioned samples comprises one copy of the target chromosome. For example, for a sample with a target chromosome concentration of one copy of the target chromosome per 50 picoliters, the methods can comprise partitioning 5 picoliters of the sample into the plurality of partitioned samples to achieve that each of at least 10% of the plurality of partitioned samples comprises one copy of the target chromosome. For example, for a sample with a target chromosome concentration of one copy of the target chromosome per 10 picoliters, the methods can comprise partitioning 1 picoliter of the sample into the plurality of partitioned samples to achieve that each of at least 10% of the plurality of partitioned samples comprises one copy of the target chromosome.

Methods for estimating copy numbers of a plurality of target chromosomes in a sample are also disclosed. For example, the sample can comprise two target chromosomes (e.g., human chromosomes 18 and 21), and the methods can be used to estimate copy number of the first target chromosome (e.g. human chromosome 18) and copy number of the second target chromosome (e.g., human chromosome 21). In some embodiments, the sample can comprise a first target chromosome, a second target chromosome, and a third target chromosome. In some embodiments, the sample can comprise N target chromosome (N is an integer greater than 1). The methods can comprise providing a sample comprising one or more copies of each of a plurality of target chromosomes, and partitioning the sample into a plurality of partitioned samples, wherein each of at least 10% of the plurality of partitioned samples comprises only one copy of each of the plurality of target chromosomes. For example, the sample can comprise one or more copies of a first target chromosome and a second target chromosome, and each of at least 10% of the plurality of partitioned samples comprises only one copy of the first target chromosome and only one copy of the second target chromosome. In some embodiments, partitioning the sample can comprise adjusting the volume of the sample to alter the concentration of each of the plurality of target chromosomes in the sample. For example, the plurality of target chromosomes can be two or more human chromosomes 1-22, X chromosome, and Y chromosome. The number of the plurality of target chromosomes can vary. In some embodiments, the number of the plurality of target chromosomes can be, can be about, can be more than, or can be at most, 1, 2, 3, 4, 5, 6, 7, 8, 9, 10, 20, 30, 40, 50, 60, 70, 80, 90, 100.

In some embodiments, the number of the plurality of target chromosomes is 2, for example the first target chromosome and the second target chromosome. The concentration of the plurality of target chromosomes is the sum of the concentration of the first target chromosome and the concentration of the second target chromosome. In some embodiments, the number of the plurality of target chromosomes is 24, for example human chromosomes 1-22, X chromosome, and Y chromosome. The concentration of the plurality of target chromosomes is the sum of the concentrations of the 24 target chromosomes.

The desired concentration of the plurality of target chromosomes can vary. In some embodiments, the desired concentration of the plurality of target chromosomes can be, can be about, can be more than, or can be at most one copy of each of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 microliters, or a number or a range between any two of these values. In some embodiments, the desired concentration of the plurality of target chromosomes can be, can be about, can be more than, or can be at most one copy of each of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 nanoliters, or a number or a range between any two of these values. In some embodiments, the desired concentration of the plurality of target chromosomes can be, can be about, can be more than, or can be at most one copy of each of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 picoliters, or a number or a range between any two of these values. In some embodiments, the desired concentration of the plurality of target chromosomes can be, can be about, can be more than, or can be at most one copy of each of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 femtoliters, or a number or a range between any two of these values.

The desired concentration of the plurality of target chromosomes can vary. In some embodiments, the desired concentration of the plurality of target chromosomes can be, can be about, can be more than, or can be at most one copy of any one of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 microliters, or a number or a range between any two of these values. In some embodiments, the desired concentration of the plurality of target chromosomes can be, can be about, can be more than, or can be at most one copy of any one of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 nanoliters, or a number or a range between any two of these values. In some embodiments, the desired concentration of the plurality of target chromosomes can be, can be about, can be more than, or can be at most one copy of any one of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 picoliters, or a number or a range between any two of these values. In some embodiments, the desired concentration of the plurality of target chromosomes can be, can be about, can be more than, or can be at most one copy of any one of the plurality of target chromosomes per 1, 2, 3, 4, 5, 6, 7, 8, 9, 10, 20, 30, 40, 50, 60, 70, 80, 90, 100, 200, 300, 400, 500, 600, 700, 800, 900, 1000 femtoliters, or a number or a range between any two of these values.

In some embodiments, for each of the plurality of target chromosomes, each of, of about, of at least, or of at most, 1%, 2%, 3%, 4%, 5%, 6%, 7%, 8%, 9%, 10%, 20%, 30%, 40%, 50%, 60%, 70%, 80%, 90%, 99.9%, or a number or a range between any two of these values, of the plurality of partitioned samples can comprise one copy of the target chromosome. In some embodiments, for each of the plurality of target chromosomes, each of at least 10% of the plurality of partitioned samples can comprise one copy of the target chromosome. For example, for partitioned samples of 10 picoliters, the desired concentration of the sample can be one copy of each of the plurality of target chromosomes per 100 picoliters to achieve that, for each of the plurality of target chromosomes, each of at least 10% of the plurality of partitioned samples comprises one copy of the target chromosome. The sample volume can be adjusted to achieve the desired chromosome concentration. In some embodiments, for each of the plurality of target chromosomes, each of at least 20% of the plurality of partitioned samples can comprise one copy of the target chromosomes. For example, for partitioned samples of 10 picoliters, the desired concentration of the sample can be two copies of each of the plurality of target chromosomes per 100 picoliters to achieve that, for each of the plurality of target chromosomes, each of at least 20% of the plurality of partitioned sample comprises one copy of the target chromosome. The sample volume can be adjusted to achieve the desired chromosome concentration. In some embodiments, for each of the plurality of target chromosomes, each of at least 30% of the plurality of partitioned samples can comprise one copy of the target chromosome. For partitioned samples of 10 picoliters, the desired concentration of the sample can be three copies of each of the plurality of target chromosomes per 100 picoliters to achieve that, for each of the plurality of target chromosomes, each of at least 30% of the plurality of partitioned sample comprises one copy of the target chromosomes. The sample volume can be adjusted to achieve the desired chromosome concentration.

In some embodiments, each of, of about, of at least, or of at most, 1%, 2%, 3%, 4%, 5%, 6%, 7%, 8%, 9%, 10%, 20%, 30%, 40%, 50%, 60%, 70%, 80%, 90%, 99.9%, or a number or a range between any two of these values, of the plurality of partitioned samples can comprise one copy of any one of the plurality of target chromosomes. In some embodiments, each of at least 10% of the plurality of partitioned samples can comprise one copy of any one of the plurality of target chromosomes. For example, for partitioned samples of 10 picoliters, the desired concentration of the sample can be one copy of any one of the plurality of target chromosomes per 100 picoliters to achieve that each of at least 10% of the plurality of partitioned sample comprises one copy of the any one of the plurality of target chromosomes. The sample volume can be adjusted to achieve the desired chromosome concentration. In some embodiments, each of at least 20% of the plurality of partitioned samples can comprise one copy of any one of the plurality of target chromosomes. For example, for partitioned samples of 10 picoliters, the desired concentration of the sample can be two copies of each of the plurality of target chromosomes per 100 picoliters to achieve that each of at least 20% of the plurality of partitioned sample comprises one copy of any one of the plurality of target chromosomes. The sample volume can be adjusted to achieve the desired chromosome concentration. In some embodiments, each of at least 30% of the plurality of partitioned samples can comprise one copy of any one of the plurality of target chromosomes. For partitioned samples of 10 picoliters, the desired concentration of the sample can be three copies of each of the plurality of target chromosomes per 100 picoliters to achieve that each of at least 30% of the plurality of partitioned sample comprises one copy of any one of the plurality of target chromosomes. The sample volume can be adjusted to achieve the desired chromosome concentration.

In some embodiments, partitioning the sample comprises adjusting the volume of the sample partitioned into each of the plurality of partitioned samples. The desired volume of the sample partitioned into each of the plurality of partitioned samples can vary. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 microliters, or a number or a range between any two of these values. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 nanoliters, or a number or a range between any two of these values. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 picoliters, or a number or a range between any two of these values. In some embodiments, the desired volume can be, can be about, can be more than, or can be at most 1, 2, 3, 4, 5, 6, 7, 8, 9, 10, 20, 30, 40, 50, 60, 70, 80, 90, 100, 200, 300, 400, 500, 600, 700, 800, 900, 1000 femtoliters, or a number or a range between any two of these values.

In some embodiments, for each of the plurality of target chromosomes, each of at least 10% of the plurality of partitioned samples can comprise one copy of the target chromosome. For example, for a sample with a concentration of one copy of each of the plurality of target chromosomes per 100 picoliters, the methods can comprise partitioning 10 picoliters of the sample into the plurality of partitioned samples to achieve that, for each of the plurality of target chromosomes, each of at least 10% of the plurality of partitioned sample comprises one copy of the target chromosome. For a sample with a concentration of one copy of each of the plurality of target chromosomes per 50 picoliters, the methods can comprise partitioning 5 picoliters of the sample into the plurality of partitioned samples to achieve that, for each of the plurality of target chromosomes, each of at least 10% of the plurality of partitioned sample comprises one copy of the target chromosome. For a sample with a chromosome concentration of one copy of each of the plurality of target chromosomes per 10 picoliters, the methods can comprise partitioning 1 picoliter of the sample into the plurality of partitioned samples to achieve that, for each of the plurality of target chromosomes, each of at least 10% of the plurality of partitioned sample comprises one copy of any one of the target chromosomes.

In some embodiments, each of at least 10% of the plurality of partitioned samples can comprise one copy of any one of the plurality of target chromosomes. For example, for a sample with a concentration of one copy of any one of the plurality of target chromosomes per 100 picoliters, the methods can comprise partitioning 10 picoliters of the sample into the plurality of partitioned samples to achieve that each of at least 10% of the plurality of partitioned sample comprises one copy of any one of the target chromosomes. For a sample with a concentration of one copy of any one of the plurality of target chromosomes per 50 picoliters, the methods can comprise partitioning 5 picoliters of the sample into the plurality of partitioned samples to achieve that each of at least 10% of the plurality of partitioned sample comprises one copy of any one of the target chromosomes. For a sample with a chromosome concentration of one copy of each of the plurality of target chromosomes per 10 picoliters, the methods can comprise partitioning 1 picoliter of the sample into the plurality of partitioned samples to achieve that each of at least 10% of the plurality of partitioned sample comprises one copy of any one of the target chromosomes.

### Chromosome Fragmentation

In some embodiments of the methods disclosed herein, a sample can comprise one or more target chromosomes, and the one or more target chromosomes can be fragmented. As illustrated in FIG. 2, at 208, stochastically barcoding one or more copies of the one or more target chromosomes can comprise fragmenting the one or more copies of the one or more target chromosomes to generate fragments of the one or more target chromosomes. For example, the sample can comprise a first target chromosome and stochastically barcoding the first target chromosome can comprise fragmenting the one or more copies of the first target chromosome to generate fragments of the first target chromosome. For example, the sample can comprise a first target chromosome and a second target chromosome, and stochastically barcoding the first target chromosome and the second target chromosome can comprise fragmenting the one or more copies of the first target chromosome and the second target chromosome to generate fragments of the first target chromosome and the second target chromosome. For example, the sample can comprise a first target chromosome, a second target chromosome, and a third target chromosome, and stochastically barcoding one or more copies of the one or more target chromosomes can comprise fragmenting the one or more copies of the first target chromosome, the second target chromosome, and the third target chromosome to generate fragments of the first target chromosome, the second target chromosome, and the third target chromosome. For example, the sample can comprise N target chromosome (N is an integer greater than 1), and stochastically barcoding one or more copies of the one or more target chromosomes can comprise fragmenting the one or more copies of each of the n target chromosomes to generate fragments of the n target chromosomes.

The length of the fragments of the one or more target chromosomes can vary. In some embodiments, the fragments of the one or more target chromosomes can be, can be about, can be at least, or can be at most, 10, 20, 30, 40, 50, 60, 70, 80, 90, 100, 200, 300, 400, 500, 600, 700, 800, 900, 1000 kilo bases, or a number or a range between any two of these values, in length.

For a target chromosome, the stochastically barcoded target chromosome can comprise stochastically barcoded fragments of the target chromosome. For example, the stochastically barcoded first target chromosome can comprise stochastically barcoded fragments of the first target chromosome. For example, the stochastically barcoded second target chromosome can comprise stochastically barcoded fragments of the first target chromosome. For example, the stochastically barcoded n^{th} target chromosome can comprise stochastically barcoded fragments of the n^{th} target chromosome.

The number of stochastically barcoded fragments of a target chromosome in the stochastically barcoded target chromosome can vary. In some embodiments, the number of stochastically barcoded fragments of the target chromosome in the stochastically barcoded target chromosome can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, 10⁴, 10⁵, 10⁶, 10⁷, or a number or a range between any two of these values.

### Haplotype Phasing

Disclosed herein are methods for haplotype phasing two or more gene targets on a target chromosome in a sample. In some embodiments, the methods comprise: providing a sample comprising one or more copies of a target chromosome, wherein the target chromosome comprises two or more gene targets; partitioning the sample into a plurality of partitioned samples, wherein each of a desirable percentage of the plurality of partitioned samples comprises one copy of the target chromosome; stochastically barcoding the one or more copies of the target chromosome in the plurality of partitioned samples using a plurality of stochastic barcodes, wherein each of the plurality of stochastic barcodes comprises a chromosome label and a molecular label; and determining the haplotype phasing of the two or more gene targets on the target chromosome in the sample using the chromosome label and the molecular label. The desirable percentage of the plurality of partitioned sample can be, can be about, can be at least, or can be at most, for example, 10%, 15%, 20%, 25%, 30%, 40%, 50%, 60%, 70%, 80%, 90%, 95%, 99%, or a number or a range between any two of these values, of the plurality of partitioned sample.

The disclosure provides for methods for stochastically labeling a sample (e.g., chromosomes), for example, for use in haplotype phasing. In some embodiments, a plurality of chromosomes from a sample (e.g., a single cell) can be distributed into microwells of a substrate, wherein the microwell comprises one chromosome. The chromosome can be contacted with a stochastic barcode. The stochastic barcode can be attached to a solid support (e.g., bead). The stochastic barcode can comprise a gene-specific region that can hybridize to a target (e.g., gene) on the chromosome. The stochastic barcode can stochastically label the chromosome.

The nucleic acid sample (e.g., a sample comprising chromosomes) can be diluted such that one chromosome is in one microwell of a substrate. In some embodiments, at least 10, 20, 30, 40, 50, 60, 70, 80, or 90% or more microwells may not comprise a chromosome. In some embodiments, at most 10, 20, 30, 40, 50, 60, 70, 80, or 90% or more microwells may not comprise a chromosome. In some embodiments, at least 10, 20, 30, 40, 50, 60, 70, 80, or 90% or more microwells may comprise a chromosome. In some embodiments, at most 10, 20, 30, 40, 50, 60, 70, 80, or 90% or more microwells may comprise a chromosome.

In some embodiments, the chromosome in the microwell can be modified prior to stochastic barcoding. The chromosome can be partially or fully unwound. The chromosome can be, for example, acetylated, methylated, deacetylated, demethylated, and the like. The chromosome can be contacted with a modifying agent (e.g., a histone modifying agent, i.e., methyltransferase, helicase, acetytransferase, etc). The chromosome can transcribed into RNA (e.g., in vitro transcription). The stochastic barcode can contact the transcribed RNA.

In some embodiments, the chromosome can be fragmented. Individual chromosome fragments can be stochastically barcoded according to the methods of the disclosure. In some embodiments, the alleles of a chromosome can be labelled. The alleles can be counted. Allelic calling can be performed. The methods can comprise determining the genotype of a target molecule (e.g., originating from a chromosome, or a cellular sample).

Methods disclosed herein can be used for haplotype analysis, haplotype construction, genetic phasing, determination of the chromosomal original of a target nucleic acid (e.g., maternal or paternal). Methods disclosed herein can be used for building diploid reference genomes. Methods disclosed herein can be used for determining structural rearrangements in a chromosome (e.g., genetic mobility event).

In some embodiments, the disclosure provides a method to determine haplotype phasing comprising a step of identifying one or more sites of heterozygosity in the plurality of read pairs, wherein phasing data for allelic variants can be determined by identifying read pairs that comprise a pair of heterozygous sites.

In some embodiments, the disclosure provides a method of haplotype phasing, comprising generating a plurality of read-pairs from a single DNA molecule and assembling a plurality of contigs of the DNA molecule using the read-pairs. In some embodiments, at least 1% of the read-pairs spans a distance greater than 50 kilo bases (kb) on the single DNA molecule and the haplotvpe phasing is performed at greater than 70% accuracy. In some embodiments, at least 10% of the read-pairs span a distance greater than 50 kilo bases (kb) on the single DNA molecule. In some embodiments, wherein at least 1% of the read-pairs span a distance greater than 100 kilo bases (kb) on the single DNA molecule. In some embodiments, the haplotype phasing is performed at greater than 90% accuracy.

In some embodiments, the disclosure provides a method of haplotype phasing, comprising generating a plurality of read-pairs from a single DNA molecule (e.g., a single chromosome) in a well and assembling a plurality of contigs of the DNA molecule using the read-pairs. In some embodiments, at least 1% of the read-pairs spans a distance greater than 30 kilo bases (kb) on the single DNA molecule and the haplotype phasing is performed at greater than 70% accuracy. In some embodiments, at least 10% of the read-pairs span a distance greater than 30 kilo bases (kb) on the single DNA molecule. In some embodiments, at least 1% of the read-pairs span a distance greater than 50 kilo bases (kb) on the single DNA molecule. In some embodiments, the haplotype phasing is performed at greater than 90% accuracy. In some embodiments, the haplotype phasing is performed at greater than 70% accuracy.

### Aneuploidy Determination

Disclosed herein are methods for determining aneuploidy of one or more cells. In some embodiments, the methods comprise: providing a sample comprising chromosomes from one or more cells; partitioning the sample into a plurality of partitioned samples, wherein each of at least 10% of the plurality of partitioned samples comprises one copy of a first target chromosome; stochastically barcoding the one or more copies of the first target chromosome in the plurality of partitioned samples using a first plurality of stochastic barcodes, wherein each of the first plurality of stochastic barcodes comprises a first chromosome label and a first molecular label; and determining the aneuploidy of the one or more cells in the sample, wherein determining the aneuploidy of the one or more cells in the sample comprises determining the number of a first gene target on the first target chromosome using the first chromosome label and the first molecular label. In some embodiments, the ancuploidy is a trisomy. The trisomy can be an autosomal trisomy.

The methods disclosed herein can be used for prenatal diagnostics. The methods and kits disclosed herein can comprise diagnosing a fetal condition in a pregnant subject. The methods and kits disclosed herein can comprise identifying fetal mutations or genetic abnormalities. Molecules (e.g., chromosomes) to be stochastically labeled may be from a fetal cell or tissue. In some embodiments, the molecules (e.g., chromosomses) to be labeled may be from the pregnant subject.

The methods and kits disclosed herein can be used in the diagnosis, prediction or monitoring of autosomal trisomies (e.g., Trisomy 13, 15, 16, 18, 21, or 22). In some cases the trisomy may be associated with an increased chance of miscarriage (e.g., Trisomy 15, 16, or 22). In some embodiments, the trisomy that is detected is a liveborn trisomy that may indicate that an infant will be born with birth defects (e.g., Trisomy 13 (Patau Syndrome), Trisomy 18 (Edwards Syndrome), and Trisomy 21 (Down Syndrome)). The abnormality may also be of a sex chromosome (e.g., XXY (Klinefelter's Syndrome), XYY (Jacobs Syndrome), or XXX (Trisomy X). The molecule(s) to be labeled may be on one or more of the following chromosomes: 13, 18, 21, X, or Y. For example, the molecule is on chromosome 21 and/or on chromosome 18, and/or on chromosome 13.

Non-limiting fetal conditions that may be determined based on the methods and kits disclosed herein include monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g., XXXX, XXYY, XXXY, XYYY, XXXXX, XXXXY, XXXYY, XYYYY and XXYYY), monoploidy, triploidy (three of every chromosome, e.g., 69 chromosomes in humans), tetraploidy (four of every chromosome, e.g., 92 chromosomes in humans), pentaploidy and multiploidy.

In some embodiments, the sample comprises one or more copies of a second target chromosome, and wherein each of at least 10% of the plurality of partitioned samples comprises one copy of the second target chromosomes, the methods further comprise: stochastically barcoding the one or more copies of the second target chromosome in the plurality of partitioned samples using a second plurality of stochastic barcodes, wherein each of the second plurality of stochastic barcodes comprises a second chromosome label and a second molecular label, wherein stochastically barcoding the one or more copies of the second target chromosome comprises fragmenting the one or more copies of the second target chromosome to generate fragments of the second target chromosome and generating an indexed library of stochastically barcoded fragments of the second target chromosome, and wherein determining the aneuploidy of the one or more cells in the sample further comprises determining the number of a second gene target on the second target chromosome using the second chromosome label and the second molecular label and comparing the number of the first gene target and the number of the second gene target.

In some embodiments, the sample comprises one or more copies of each of n target chromosomes, wherein n is an integer greater than one, and wherein each of the plurality of partitioned samples comprises one copy of each of the n target chromosomes, the methods further comprise: for each of the n target chromosomes in the plurality of partitioned samples, stochastically barcoding the one or more copies of the n^{th} target chromosome using a n^{th} plurality of stochastic barcodes, wherein each of the n^{th} stochastic barcodes comprises a n^{th} chromosome label and a n^{th} molecular label, wherein stochastically barcoding the one or more copies of the n^{th} target chromosome comprises fragmenting the one or more copies of the n^{th} target chromosome to generate fragments of the n^{th} target chromosome and generating an indexed library of stochastically barcoded fragments of the n^{th} target chromosome, and wherein determining the aneuploidy of the one or more cells in the sample further comprises, for each of n target chromosomes, determining the number of a n^{th} gene target on the n^{th} target chromosome in the indexed library and comparing the number of the first gene target and the number of the n^{th} gene target.

### Sequence Determination

Disclosed herein are methods for sequencing a first target chromosome in a sample. In some embodiments, the methods comprise: providing a sample comprising one or more copies of a first target chromosome, partitioning the sample into a plurality of partitioned samples, wherein each of at least 10% of the plurality of partitioned samples comprises one copy of the first target chromosome; stochastically barcoding the one or more copies of the first target chromosome in the plurality of partitioned samples using a plurality of stochastic barcodes, wherein each of the plurality of stochastic barcodes comprises a chromosome label and a molecular label; and obtaining sequence information of the first target chromosome using the chromosome label and the molecular label. The methods can be used for whole genome sequencing.

The disclosure provides methods for greatly accelerating and improving de novo genome assembly. The methods disclosed herein can utilize methods for data analysis that allow for rapid and inexpensive de novo assembly of genomes from one or more subjects.

In some embodiments, obtaining the sequence information of the first target chromosome comprises determining sequences of at least some of the stochastically barcoded fragments in the indexed library. Determining the sequences of the at least some of the stochastically barcoded fragments of the first target chromosome in the indexed library can comprise generating sequences. Read lengths of the sequences generated can vary. In some embodiments, read lengths can be, can be about, can be at least, or can be at most, 10, 20, 30, 40, 50, 60, 70, 80, 90, 100, 200, 300, 400, 500, 600, 700, 800, 900, 1000, 10⁴, 10⁵, 10⁶, 10⁷, 10⁸, 10⁹, 10¹⁰, or a number or a range between any two of these values, bases.

Sequencing the at least some of the stochastically barcoded fragments in the indexed library can comprise deconvoluting the sequencing result from sequencing the indexed library. Deconvoluting the sequencing result can comprise using a software-as-a-service platform. In some embodiments, the sample comprises one or more copies of a second target chromosome, and wherein each of at least 10% of the plurality of partitioned samples comprises one copy of the second target chromosome, the method further comprise: stochastically barcoding the one or more copies of the second target chromosome in the plurality of partitioned samples using a second plurality of stochastic barcodes, wherein each of the second plurality of stochastic barcodes comprises a second chromosome label and a second molecular label, and wherein the first chromosome labels of the first plurality of stochastic barcodes and the second chromosome labels of the second plurality of stochastic barcodes differ by at least one nucleotide, wherein stochastically barcoding the one or more copies of the second target chromosome comprises fragmenting the one or more copies of the second target chromosome to generate fragments of the second target chromosome and generating an indexed library of stochastically barcoded fragments of the second target chromosome; obtaining sequence information of the second target chromosome using the second chromosome label and the second molecular label, wherein obtaining sequence information of the second target chromosome comprises determining sequences of at least some of the stochastically barcoded fragments of the second target chromosome in the indexed library.

In some embodiments, the sample comprises one or more copies of each of n target chromosomes, and wherein, for each of the n target chromosomes, each of at least 10% of the plurality of partitioned samples comprises one copy of the n^{th} target chromosome, the method further comprises: for each of the n target chromosomes, stochastically barcoding the one or more copies of the n^{th} target chromosome in the plurality of partitioned samples using a n^{th} plurality of stochastic barcodes, wherein each of the n^{th} plurality of stochastic barcodes comprises a n^{th} chromosome label and a n^{th} molecular label, and wherein the first chromosome labels of the first plurality of stochastic barcodes and the n^{th} chromosome labels of the nm plurality of stochastic barcodes differ by at least one nucleotide, and wherein stochastically barcoding the one or more copies of the n^{th} target chromosome comprises fragmenting the one or more copies of the n^{th} target chromosome to generate fragments of the n^{th} target chromosome and generating an indexed library of stochastically barcoded fragments of the n^{th} target chromosome, for each of the n target chromosomes, obtaining sequence information of the n^{t}h target chromosome using the n^{th} chromosome label and the n^{th} molecular label, wherein obtaining sequence information of the n^{th} target chromosome comprises determining sequences of at least some of the stochastically barcoded fragments of n^{th} target chromosome in the indexed library.

In some embodiments, obtaining the sequence information of a target chromosome, for example the first target chromosome or the second target chromosome, can comprise obtaining the sequence information of at least 10% of the base pairs of the target chromosome. Sequence information of different percentages of the base pairs of the target chromosome can be obtained. In some embodiments, the percentage of the base pairs of the target chromosome with obtained sequence information can be, can be about, can be at least, or can be at most, 0.0001%, 0.001%, 0.01%, 0.1%, 1%, 2%, 3%, 4%, 5%, 6%, 7%, 8%, 9%, 10%, 20%, 30%, 40%, 50%, 60%, 70%, 80%, 90%, 99%, 99.9%, or a number or a range between any two of these values, of the base pairs of the target chromosome. In some embodiments, the number of the base pairs of the target chromosome with obtained sequence information can be, can be about, can be at least, or can be at most, 100, 200, 300, 400, 500, 600, 700, 800, 900, 1000 base pairs, or a number or a range between any two of these values. In some embodiments, the number of the base pairs of the target chromosome with obtained sequence information can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000 kilo base pairs (kbp), or a number or a range between any two of these values. In some embodiments, the number of the base pairs of the target chromosome with obtained sequence information can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000 mega base pairs (Mbp), or a number or a range between any two of these values.

### Solid Supports

Stochastic barcodes disclosed herein can, in some embodiments, be associated with a solid support. The solid support can be, for example, a synthetic particle. In some embodiments, some or all of the molecular labels (e.g., the first molecular labels) of a plurality of stochastic barcodes (e.g., the first plurality of stochastic barcodes) on a solid support differ by at least one nucleotide. The chromosome labels of the stochastic barcodes on the same solid support can be the same. The chromosome labels of the stochastic barcodes on different solid supports can differ by at least one nucleotide. For example, first chromosome labels of a first plurality of stochastic barcodes on a first solid support can have the same sequence, and second chromosome labels of a second plurality of stochastic barcodes on a second solid support can have the same sequence. The first chromosome labels of the first plurality of stochastic barcodes on the first solid support and the second chromosome labels of the second plurality of stochastic barcodes on the second solid support can differ by at least one nucleotide. A chromosome label can be, for example, about 5-20 nucleotides long. A molecular label can be, for example, about 5-20 nucleotides long. The synthetic particle can be, for example, a bead. The bead can be, for example, a silica gel bead, a controlled pore glass bead, a magnetic bead, a Dynabead, a Sephadex/Sepharose bead, a cellulose bead, a polystyrene bead, or any combination thereof.

For example, in a non-limiting example of stochastic barcoding illustrated in FIG. 2, at 212, beads can be introduced onto the plurality of microwells of the well array. Each microwell can comprise one bead. The beads can comprise a plurality of stochastic barcodes. A stochastic barcode can comprise a 5′ amine region attached to a bead. The stochastic barcode can comprise a universal label, a molecular label, a target-binding region, or any combination thereof.

The stochastic barcodes disclosed herein can be associated to (e.g., attached to) a solid support (e.g., a bead). In some embodiments, stochastically barcoding the plurality of targets in the sample can be performed with a solid support including a plurality of synthetic particles associated with the plurality of stochastic barcodes. In some embodiments, the solid support can include a plurality of synthetic particles associated with the plurality of stochastic barcodes. The spatial labels of the plurality of stochastic barcodes on different solid supports can differ by at least one nucleotide. The solid support can, for example, include the plurality of stochastic barcodes in two dimensions or three dimensions. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can include a polymer, a matrix, a hydrogel, a needle array device, an antibody, or any combination thereof. In some embodiments, the solid supports can be free floating. In some embodiments, the solid supports can be embedded in a semi-solid or solid array. The stochastic barcodes may not be associated with solid supports. The stochastic barcodes can be individual nucleotides. The stochastic barcodes can be associated with a substrate.

As used herein, the terms “tethered”, “attached”, and “immobilized” are used interchangeably, and can refer to covalent or non-covalent means for attaching stochastic barcodes to a solid support. Any of a variety of different solid supports can be used as solid supports for attaching pre-synthesized stochastic barcodes or for in situ solid-phase synthesis of stochastic barcode.

In some embodiments, the solid support is a bead. The bead can comprise one or more types of solid, porous, or hollow sphere, ball, bearing, cylinder, or other similar configuration which a nucleic acid can be immobilized (e.g., covalently or non-covalently). The bead can be, for example, composed of plastic, ceramic, metal, polymeric material, or any combination thereof. A bead can be, or comprise, a discrete particle that is spherical (e.g., microspheres) or have a non-spherical or irregular shape, such as cubic, cuboid, pyramidal, cylindrical, conical, oblong, or disc-shaped, and the like. In some embodiments, a bead can be non-spherical in shape.

Beads can comprise a variety of materials including, but not limited to, paramagnetic materials (e.g. magnesium, molybdenum, lithium, and tantalum), superparamagnetic materials (e.g. ferrite (Fe₃O₄; magnetite) nanoparticles), ferromagnetic materials (e.g. iron, nickel, cobalt, some alloys thereof, and some rare earth metal compounds), ceramic, plastic, glass, polystyrene, silica, methylstyrene, acrylic polymers, titanium, latex, sepharose, agarose, hydrogel, polymer, cellulose, nylon, and any combination thereof.

The diameter of the beads can vary, for example, be, be at least, or be at least about, 100 nm, 500 nm, 1 μm, 5 μm, 10 μm, 20 μm, 25 μm, 30 μm, 35 μm, 40 μm, 45 μm, 50 μm, or a number or a range between any two of these values. In some embodiments, the diameter of the beads can be, be at most, or be at most about, 100 nm, 500 nm, 1 μm, 5 μm, 10 μm, 20 μm, 25 μm, 30 μm, 35 μm, 40 μm, 45 μm, 50 μm, or a number or a range between any two of these values. In some embodiments, the diameter of the bead can be related to the diameter of the wells of the substrate. For example, the diameter of the bead can be, be at least, or be at least about, 10%, 20%, 30%, 40%, 50%, 60%, 70%, 80%, 90%, 100%, or a number or a range between any two of these values, longer or shorter than the diameter of the well. In some embodiments, the diameter of the bead can be, be at most, or be at most about, 10%, 20%, 30%, 40%, 50%, 60%, 70%, 80%, 90%, 100%, or a number or a range between any two of these values, longer or shorter than the diameter of the well. The diameter of the bead can be related to the diameter of a cell (e.g., a single cell entrapped by a well of the substrate). The diameter of the bead can be, be at least, or be at least about, 10%, 20%, 30%, 40%, 50%, 60%, 70%, 80%, 90%, 100%, 150%, 200%, 250%, 300%, or a number or a range between any two of these values, longer or shorter than the diameter of the cell. The diameter of the bead can be, be at most, or be at most, 10%, 20%, 30%, 40%, 50%, 60%, 70%, 80%, 90%, 100%, 150%, 200%, 250%, 300%, or a number or a range between any two of these values, longer or shorter than the diameter of the cell.

A bead can be attached to and/or embedded in a substrate. A bead can be attached to and/or embedded in a gel, hydrogel, polymer and/or matrix. The spatial position of a bead within a substrate (e.g., gel, matrix, scaffold, or polymer) can be identified using the spatial label present on the stochastic barcode on the bead which can serve as a location address.

Examples of beads can include, but are not limited to, streptavidin beads, agarose beads, magnetic beads, Dynabeads®, MACS, microbeads, antibody conjugated beads (e.g., anti-immunoglobulin microbeads), protein A conjugated beads, protein G conjugated beads, protein A/G conjugated beads, protein L conjugated beads, oligo(dT) conjugated beads, silica beads, silica-like beads, anti-biotin microbeads, anti-fluorochrome microbeads, and BcMag™ Carboxyl-Terminated Magnetic Beads.

A bead can be associated with (e.g. impregnated with) quantum dots or fluorescent dyes to make it fluorescent in one fluorescence optical channel or multiple optical channels. A bead can be associated with iron oxide or chromium oxide to make it paramagnetic or ferromagnetic. Beads can be identifiable. For example, a bead can be imaged using a camera. A bead can have a detectable code associated with the bead. For example, a bead can comprise a stochastic barcode. A bead can change size, for example due to swelling in an organic or inorganic solution. A bead can be hydrophobic. A bead can be hydrophilic. A bead can be biocompatible.

A solid support (e.g., bead) can be visualized. The solid support can comprise a visualizing tag (e.g., fluorescent dye). A solid support (e.g., bead) can be etched with an identifier (e.g., a number). The identifier can be visualized through imaging the beads.

A solid support can refer to an insoluble, semi-soluble, or insoluble material. A solid support can be referred to as “functionalized” when it includes a linker, a scaffold, a building block, or other reactive moiety attached thereto, whereas a solid support can be “nonfunctionalized” when it lack such a reactive moiety attached thereto. The solid support can be employed free in solution, such as in a microtiter well format; in a flow-through format, such as in a column; or in a dipstick.

The solid support can comprise a membrane, paper, plastic, coated surface, flat surface, glass, slide, chip, or any combination thereof. A solid support can take the form of resins, gels, microspheres, or other geometric configurations. A solid support can comprise silica chips, synthetic particles, nanoparticles, plates, and arrays. Solid supports can include beads (e.g., silica gel, controlled pore glass, magnetic beads, Dynabeads, Wang resin; Merrifield resin, Sephadex/Sepharose beads, cellulose beads, polystyrene beads etc.), capillaries, flat supports such as glass fiber filters, glass surfaces, metal surfaces (steel, gold silver, aluminum, silicon and copper), glass supports, plastic supports, silicon supports, chips, filters, membranes, microwell plates, slides, or the like. plastic materials including multiwell plates or membranes (e.g., formed of polyethylene, polypropylene, polyamide, polyvinylidene difluoride), wafers, combs, pins or needles (e.g., arrays of pins suitable for combinatorial synthesis or analysis) or beads in an array of pits or nanoliter wells of flat surfaces such as wafers (e.g., silicon wafers), wafers with pits with or without filter bottoms.

In some embodiments stochastic barcodes of the disclosure can be attached to a polymer matrix (e.g., gel, hydrogel). The polymer matrix can be able to permeate intracellular space (e.g., around organelles). The polymer matrix can able to be pumped throughout the circulatory system.

A solid support can be a biological molecule. For example a solid support can be a nucleic acid, a protein, an antibody, a histone, a cellular compartment, a lipid, a carbohydrate, and the like. Solid supports that are biological molecules can be amplified, translated, transcribed, degraded, and/or modified (e.g., pegylated, sumoylated). A solid support that is a biological molecule can provide spatial and time information in addition to the spatial label that is attached to the biological molecule. For example, a biological molecule can comprise a first confirmation when unmodified, but can change to a second confirmation when modified. The different conformations can expose stochastic barcodes of the disclosure to targets. For example, a biological molecule can comprise stochastic barcodes that are inaccessible due to folding of the biological molecule. Upon modification of the biological molecule (e.g., acetylation), the biological molecule can change conformation to expose the stochastic labels. The timing of the modification can provide another time dimension to the method of stochastic barcoding of the disclosure.

In another example, the biological molecule comprising stochastic barcodes of the disclosure can be located in the cytoplasm of a cell. Upon activation, the biological molecule can move to the nucleus, whereupon stochastic barcoding can take place. In this way, modification of the biological molecule can encode additional space-time information for the targets identified by the stochastic barcodes.

A dimension label can provide information about space-time of a biological event (e.g., cell division). For example, a dimension label can be added to a first cell, the first cell can divide generating a second daughter cell, the second daughter cell can comprise all, some or none of the dimension labels. The dimension labels can be activated in the original cell and the daughter cell. In this way, the dimension label can provide information about time of stochastic barcoded in distinct spaces.

### Substrates

As used herein, a substrate can refer to a type of solid support. A substrate can refer to a solid support that can comprise stochastic barcodes of the disclosure. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. In some embodiments, a microwell can entrap only one solid support. In some embodiments, a microwell entraps a single cell and a single solid support (e.g., bead).

The microwells of the array can be fabricated in a variety of shapes and sizes. Appropriate well geometries can include, but are not limited to, cylindrical, conical, hemispherical, rectangular, or polyhedral (e.g., three dimensional geometries comprised of several planar faces, for example, hexagonal columns, octagonal columns, inverted triangular pyramids, inverted square pyramids, inverted pentagonal pyramids, inverted hexagonal pyramids, or inverted truncated pyramids). The microwells can comprise a shape that combines two or more of these geometries. For example, a microwell can be partly cylindrical, with the remainder having the shape of an inverted cone. A microwell can include two side-by-side cylinders, one of larger diameter (e.g. that corresponds roughly to the diameter of the beads) than the other (e.g. that corresponds roughly to the diameter of the cells), that are connected by a vertical channel (that is, parallel to the cylinder axes) that extends the full length (depth) of the cylinders. The opening of the microwell can be at the upper surface of the substrate. The opening of the microwell can be at the lower surface of the substrate. The closed end (or bottom) of the microwell can be flat. The closed end (or bottom) of the microwell can have a curved surface (e.g., convex or concave). The shape and/or size of the microwell can be determined based on the types of cells or solid supports to be trapped within the microwells.

Microwell dimensions can be characterized in terms of the diameter and depth of the well. As used herein, the diameter of the microwell refers to the largest circle that can be inscribed within the planar cross-section of the microwell geometry. The diameter of the microwells can range from about 1-fold to about 10-folds the diameter of the cells or solid supports to be trapped within the microwells. The microwell diameter can be at least 1-fold, at least 1.5-fold, at least 2-folds, at least 3-folds, at least 4-folds, at least 5-folds, or at least 10-folds the diameter of the cells or solid supports to be trapped within the microwells. The microwell diameter can be at most 10-folds, at most 5-folds, at most 4-folds, at most 3-folds, at most 2-folds, at most 1.5-fold, or at most 1-fold the diameter of the cells or solid supports to be trapped within the microwells. The microwell diameter can be about 2.5-folds the diameter of the cells or solid supports to be trapped within the microwells.

The diameter of the microwells can be specified in terms of absolute dimensions. The diameter of the microwells can range from about 5 to about 50 micrometers. The microwell diameter can be, can be at least, or can be at least about, 5, 10, 15, 20, 25, 30, 35, 40, 45, 50 micrometers, or a number or a range between any two of these values. The microwell diameter can be, can be at most, or can be at most about, 50, 45, 40, 35, 30, 25, 20, 15, 10, 5 micrometers, or a number or a range between any two of these values. The microwell diameter can be about 30 micrometers.

In some embodiments, the diameter of each microwell can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, nanometers. In some embodiments, the diameter of each microwell can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, micrometers. In some embodiments, the diameter of each microwell can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, minimeters.

The microwell depth can be chosen to provide efficient trapping of cells and solid supports. The microwell depth can be chosen to provide efficient exchange of assay buffers and other reagents contained within the wells. The ratio of diameter to height (i.e. aspect ratio) can be chosen such that once a cell and solid support settle inside a microwell, they will not be displaced by fluid motion above the microwell. In some embodiments, the height of the microwell can be smaller than the diameter of the bead. For example, the height of the microwell can be at least 5, 10, 15, 20, 25, 30, 35, 40, 45, 50, 55, 60, 65, 70, 75, 80, 85, 90, 95, 100%, or a number or a range between any two of these values, of the diameter of the bead. The bead can protrude outside of the microwell.

The dimensions of the microwell can be chosen such that the microwell has sufficient space to accommodate a solid support and a cell of various sizes without being dislodged by fluid motion above the microwell. The depth of the microwells can range from about 1-fold to about 10-folds the diameter of the cells or solid supports to be trapped within the microwells. The microwell depth can be at least 1-fold, at least 1.5-fold, at least 2-folds, at least 3-folds, at least 4-folds, at least 5-folds, or at least 10-folds the diameter of the cells or solid supports to be trapped within the microwells. The microwell depth can be at most 10-folds, at most 5-folds, at most 4-folds, at most 3-folds, at most 2-folds, at most 1.5-fold, or at most 1-fold the diameter of the cells or solid supports to be trapped within the microwells. The microwell depth can be about 2.5-folds the diameter of the cells or solid supports to be trapped within the microwells.

The depth of the microwells can be specified in terms of absolute dimensions. The depth of the microwells can range from about 10 micrometers to about 60 micrometers. The microwell depth can be at least 10, 15, 20, 25, 30, 35, 40, 45, 50, 55, 60, micrometers, or a number or a range between any two of these values. The microwell depth can be at most 60, 55, 50, 45, 40, 35, 30, 25, 20, 15, 10 micrometers, or a number or a range between any two of these values. The microwell depth can be about 30 micrometers.

In some embodiments, the depth of each microwell can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, nanometers. In some embodiments, the depth of each microwell can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, micrometers. In some embodiments, the depth of each microwell can be, can be about, can be at least, or can be at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, minimeters.

The volume of the microwells used in the methods, devices, and systems of the present disclosure can vary, for example ranging from about 200 micrometers³ to about 120,000 micrometers³. The microwell volume can be, can be about, can be at least, or can be at least about 200, 500, 1000, 10000, 25000, 50000, 100000, 120000 micrometers³, or a number or a range between any two of these values. The microwell volume can be, can be at most, or can be at most about, 120000, 100000, 50000, 25000, 10000, 1000, 500, 200 micrometers³, or a number or a range between any two of these values. The microwell volume can be about 25,000 micrometers³. The microwell volume can fall within any range bounded by any of these values (e.g. from about 18,000 micrometers³ to about 30,000 micrometers³).

In some embodiments, each of the microwells can have a volume of, of about, of at least, or of at most, 10, 20, 30, 40, 50, 60, 70, 80, 90, 100, 200, 300, 400, 500, 600, 700, 800, 900, 1000, or a number or a range between any two of these values, nanoliters. In some embodiments, each of the microwells can have a volume of, of about, of at least, or of at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, microliters. In some embodiments, each of the microwells can have a volume of, of about, of at least, or of at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, miniliters.

The volumes of the microwells used in the methods, devices, and systems of the present disclosure can be further characterized in terms of the variation in volume from one microwell to another. The coefficient of variation (expressed as a percentage) for microwell volume can range from about 1% to about 10%. The coefficient of variation for microwell volume can be, can be about, can be at least, or can be at least about, 1%, 2%, 3%, 4%, 5%, 6%, 7%, 8%, 9%,10%, or a number or a range between any two of these values. The coefficient of variation for microwell volume can be, can be at most, or can be at most about, 10%, 9%, 8%, 7%, 6%, 5%, 4%, 3%, 2%, 1%, or a number or a range between any two of these values. The coefficient of variation for microwell volume can have any value within a range encompassed by these values, for example between about 1.5% and about 6.5%. In some embodiments, the coefficient of variation of microwell volume can be about 2.5%.

The ratio of the volume of the microwells to the surface area of the beads (or to the surface area of a solid support to which stochastic barcode oligonucleotides can be attached) used in the methods, devices, and systems of the present disclosure can vary, for example range from about 2.5 to about 1520 micrometers. The ratio can be, can be about, can be at least, or can be at least about 2.5, 5, 10, 100, 500, 750, 1000, 1520 micrometers, or a number or a range between any two of these values. The ratio can be, can be at most, or can be at most about, 1520, 1000, 750, 500, 100, 10, 5, 2 micrometers, or a number or a range between any two of these values. In some embodiments, the ratio can be, be about, be at least, or be at most, 67.5 micrometers. The ratio of microwell volume to the surface area of the bead (or solid support used for immobilization) can fall within any range bounded by any of these values (e.g. from about 30 to about 120).

The wells of the microwell array can be arranged in a one dimensional, two dimensional, or three-dimensional array. A three dimensional array can be achieved, for example, by stacking a series of two or more two dimensional arrays (that is, by stacking two or more substrates comprising microwell arrays).

The pattern and spacing between microwells can be chosen to optimize the efficiency of trapping a single cell and single solid support (e.g., bead) in each well, as well as to maximize the number of wells per unit area of the array. The microwells can be distributed according to a variety of random or non-random patterns. For example, they can be distributed entirely randomly across the surface of the array substrate, or they can be arranged in a square grid, rectangular grid, hexagonal grid, or the like. The center-to-center distance (or spacing) between wells can vary from about 15 micrometers to about 75 micrometers. In other embodiments, the spacing between wells is, is about, is at least, or is at least about, 15, 20, 25, 30, 35, 40, 45, 50, 55, 60, 65, 70, 75 micrometers, or a number or a range between any two of these values. The microwell spacing can be, can be at most, or can be at most about, 75, 70, 65, 60, 55, 50, 45, 40, 35, 30, 25, 20, 15 micrometers, or a number or a range between any two of these values. The microwell spacing can be about 55 micrometers. The microwell spacing can fall within any range bounded by any of these values (e.g. from about 18 micrometers to about 72 micrometers).

In some embodiments, microwells can be separated from each other by no more than 0.01, 0.1, 1, 2, 3, 4, 5, 6, 7, 8, 9, 10, 20, 30, 40, 50, 60, 70, 80, 90, 100, 200, 300, 400, 500, 600, 700, 800, 900, 1000, or a number between any two of these values, micrometers. In some embodiments, the microwells can be separated from one another by no more than 1, 2, 3, 4, 5, 6, 7, 8, 9, 10, 20, 30, 40, 50, 60, 70, 80, 90, 100, 200, 300, 400, 500, 600, 700, 800, 900, 1000, or a number between any two of these values, minimeters.

In some embodiments, the microwell array can comprise 100, 200, 300, 400, 500, 600, 700, 800, 900, 1000, 2000, 3000, 4000, 5000, 6000, 7000, 8000, 9000, 10000, or a number between any two of these values, wells per inch². In some embodiments, the microwell array can comprise 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or a number between any two of these values, wells per cm².

The microwell array can comprise surface features between the microwells that are designed to help guide cells and solid supports into the wells and/or prevent them from settling on the surfaces between wells. Examples of suitable surface features can include, but are not limited to, domed, ridged, or peaked surface features that encircle the wells or straddle the surface between wells.

The total number of wells in the microwell array can be determined by the pattern and spacing of the wells and the overall dimensions of the array. The number of microwells in the array can vary, for example, ranging from about 96 to about 5000000. The number of microwells in the array can be, can be about, can be at least, or can be at least about 96, 384, 1536, 5000, 10000, 25000, 50000, 75000, 100000, 500000, 1000000, 5000000, or a number or a range between any two of these values. The number of microwells in the array can be, can be at most, or can be at most about 5000000, 1000000, 75000, 50000, 25000, 10000, 5000, 1536, 384, 96, or a number or a range between any two of these values. The number of microwells in the array can be, can be about, can be at least, or can be at most, 96. The number of microwells can be, can be about, can be at least, or can be at most, 150000. The number of microwells in the array can fall within any range bounded by any of these values (e.g. from about 100 to 325000).

Microwell arrays can be fabricated using any of a number of fabrication techniques. Examples of fabrication methods that can be used include, but are not limited to, bulk micromachining techniques such as photolithography and wet chemical etching, plasma etching, or deep reactive ion etching; micro-molding and micro-embossing; laser micromachining; 3D printing or other direct write fabrication processes using curable materials; and similar techniques.

Microwell arrays can be fabricated from any of a number of substrate materials. The choice of material can depend on the choice of fabrication technique, and vice versa. Examples of suitable materials can include, but are not limited to, silicon, fused-silica, glass, polymers (e.g. agarose, gelatin, hydrogels, polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, thiol-ene based resins, metals or metal films (e.g. aluminum, stainless steel, copper, nickel, chromium, and titanium), and the like. A hydrophilic material can be desirable for fabrication of the microwell arrays (e.g. to enhance wettability and minimize non-specific binding of cells and other biological material). Hydrophobic materials that can be treated or coated (e.g. by oxygen plasma treatment, or grafting of a polyethylene oxide surface layer) can also be used. The use of porous, hydrophilic materials for the fabrication of the microwell array can be desirable in order to facilitate capillary wicking/venting of entrapped air bubbles in the device. The microwell array can be fabricated from a single material. The microwell array can comprise two or more different materials that have been bonded together or mechanically joined.

Microwell arrays can be fabricated using substrates of any of a variety of sizes and shapes. For example, the shape (or footprint) of the substrate within which microwells are fabricated can be square, rectangular, circular, or irregular in shape. The footprint of the microwell array substrate can be similar to that of a microtiter plate. The footprint of the microwell array substrate can be similar to that of standard microscope slides, e.g. about 75 mm long×25 mm wide (about 3″ long×1″ wide), or about 75 mm long×50 mm wide (about 3″ long×2″ wide). The thickness of the substrate within which the microwells are fabricated can range from about 0.1 mm thick to about 10 mm thick, or more. The thickness of the microwell array substrate can be, can be about, can be at least, or can be at least about 0.1, 0.5, 1, 2, 3, 4, 5, 6, 7, 8, 9, 10 mm, or a number or a range between any two of these values. The thickness of the microwell array substrate can be, can be at most, or can be at most about, 10, 9, 8, 7, 6, 5, 4, 3, 2, 1, 0.5, 0.1 mm, or a number or a range between any two of these values. The thickness of the microwell array substrate can be about 1 mm thick. The thickness of the microwell array substrate can be any value within these ranges, for example, the thickness of the microwell array substrate can be between about 0.2 mm and about 9.5 mm.

A variety of surface treatments and surface modification techniques can be used to alter the properties of microwell array surfaces. Examples can include, but are not limited to, oxygen plasma treatments to render hydrophobic material surfaces more hydrophilic, the use of wet or dry etching techniques to smooth (or roughen) glass and silicon surfaces, adsorption or grafting of polyethylene oxide or other polymer layers (such as pluronic), or bovine serum albumin to substrate surfaces to render them more hydrophilic and less prone to non-specific adsorption of biomolecules and cells, the use of silane reactions to graft chemically-reactive functional groups to otherwise inert silicon and glass surfaces, etc. Photodeprotection techniques can be used to selectively activate chemically-reactive functional groups at specific locations in the array structure, for example, the selective addition or activation of chemically-reactive functional groups such as primary amines or carboxyl groups on the inner walls of the microwells can be used to covalently couple oligonucleotide probes, peptides, proteins, or other biomolecules to the walls of the microwells. The choice of surface treatment or surface modification utilized can depend both or either on the type of surface property that is desired and on the type of material from which the microwell array is made.

The openings of microwells can be sealed, for example, during cell lysis steps to prevent cross hybridization of target nucleic acid between adjacent microwells. A microwell (or array of microwells) can be sealed or capped using, for example, a flexible membrane or sheet of solid material (i.e. a plate or platten) that clamps against the surface of the microwell array substrate, or a suitable bead, where the diameter of the bead is larger than the diameter of the microwell.

A seal formed using a flexible membrane or sheet of solid material can comprise, for example, inorganic nanopore membranes (e.g., aluminum oxides), dialysis membranes, glass slides, coverslips, elastomeric films (e.g. PDMS), or hydrophilic polymer films (e.g., a polymer film coated with a thin film of agarose that has been hydrated with lysis buffer).

Solid supports (e.g., beads) used for capping the microwells can comprise any of the solid supports (e.g., beads) of the disclosure. In some embodiments, the solid supports are cross-linked dextran beads (e.g., Sephadex). Cross-linked dextran can range from about 10 micrometers to about 80 micrometers. The cross-linked dextran beads used for capping can be from 20 micrometers to about 50 micrometers. In some embodiments, the beads can be at least about 10, 20, 30, 40, 50, 60, 70, 80 or 90% larger than the diameter of the microwells. The beads used for capping can be at most about 10, 20, 30, 40, 50, 60, 70, 80 or 90% larger than the diameter of the microwells.

The seal or cap can allow buffer to pass into and out of the microwell, while preventing macromolecules (e.g., nucleic acids) from migrating out of the well. A macromolecule of at least about 1, 2, 3, 4, 5, 6, 7, 8, 9, 10, 12, 13, 14, 15, 16, 17, 18, 19, or 20 or more nucleotides can be blocked from migrating into or out of the microwell by the seal or cap. A macromolecule of at most about 1, 2, 3, 4, 5, 6, 7, 8, 9, 10, 12, 13, 14, 15, 16, 17, 18, 19, or 20 or more nucleotides can be blocked from migrating into or out of the microwell by the seal or cap.

Solid supports (e.g., beads) can be distributed among a substrate. Solid supports (e.g., beads) can be distributed among wells of the substrate, removed from the wells of the substrate, or otherwise transported through a device comprising one or more microwell arrays by means of centrifugation or other non-magnetic means. A microwell of a substrate can be pre-loaded with a solid support. A microwell of a substrate can hold at least 1, 2, 3, 4, or 5, or more solid supports. A microwell of a substrate can hold at most 1, 2, 3, 4, or 5 or more solid supports. In some embodiments, a microwell of a substrate can hold one solid support.

Individual cells and beads can be compartmentalized using alternatives to microwells, for example, a single solid support and single cell could be confined within a single droplet in an emulsion (e.g. in a droplet digital microfluidic system).

Cells could potentially be confined within porous beads that themselves comprise the plurality of tethered stochastic barcodes. Individual cells and solid supports can be compartmentalized in any type of container, microcontainer, reaction chamber, reaction vessel, or the like.

Single cell, stochastic barcoding or can be performed without the use of microwells. Single cell, stochastic barcoding assays can be performed without the use of any physical container. For example, stochastic barcoding without a physical container can be performed by embedding cells and beads in close proximity to each other within a polymer layer or gel layer to create a diffusional barrier between different cell/bead pairs. In another example, stochastic barcoding without a physical container can be performed in situ, in vivo, on an intact solid tissue, on an intact cell, and/or subcellularly.

Microwell arrays can be a consumable component of the assay system. Microwell arrays can be reusable. Microwell arrays can be configured for use as a stand-alone device for performing assays manually, or they can be configured to comprise a fixed or removable component of an instrument system that provides for full or partial automation of the assay procedure. In some embodiments of the disclosed methods, the bead-based libraries of stochastic barcodes can be deposited in the wells of the microwell array as part of the assay procedure. In some embodiments, the beads can be pre-loaded into the wells of the microwell array and provided to the user as part of, for example, a kit for performing stochastic barcoding and digital counting of nucleic acid targets.

In some embodiments, two mated microwell arrays can be provided, one pre-loaded with beads which are held in place by a first magnet, and the other for use by the user in loading individual cells. Following distribution of cells into the second microwell array, the two arrays can be placed face-to-face and the first magnet removed while a second magnet is used to draw the beads from the first array down into the corresponding microwells of the second array, thereby ensuring that the beads rest above the cells in the second microwell array and thus minimizing diffusional loss of target molecules following cell lysis, while maximizing efficient attachment of target molecules to the stochastic barcodes on the bead.

In some embodiments, a substrate does not include microwells. For example, beads can be assembled (e.g., self-assembled). The beads can self-assemble into a monolayer. The monolayer can be on a flat surface of the substrate. The monolayer can be on a curved surface of the substrate. The bead monolayer can be formed by any method, such as alcohol evaporation.

### Three-Dimensional Substrates

A three-dimensional substrate can be any shape. A three-dimensional substrate can be made of any material used in a substrate of the disclosure. In some embodiments, a three-dimensional substrate comprises a DNA origami. DNA origami structures incorporate DNA as a building material to make nanoscale shapes. The DNA origami process can involve the folding of one or more long, “scaffold” DNA strands into a particular shape using a plurality of rationally designed “staple DNA strands. The sequences of the staple strands can be designed such that they hybridize to particular portions of the scaffold strands and, in doing so, force the scaffold strands into a particular shape. The DNA origami can include a scaffold strand and a plurality of rationally designed staple strands. The scaffold strand can have any sufficiently non-repetitive sequence.

The sequences of the staple strands can be selected such that the DNA origami has at least one shape to which stochastic labels can be attached. In some embodiments, the DNA origami can be of any shape that has at least one inner surface and at least one outer surface. An inner surface can be any surface area of the DNA origami that is sterically precluded from interacting with the surface of a sample, while an outer surface is any surface area of the DNA origami that is not sterically precluded from interacting with the surface of a sample. In some embodiments, the DNA origami has one or more openings (e.g., two openings), such that an inner surface of the DNA origami can be accessed by particles (e.g., solid supports). For example, in certain embodiments the DNA origami has one or more openings that allow particles smaller than 10 micrometers, 5 micrometers, 1 micrometer, 500 nm, 400 nm, 300 nm, 250 nm, 200 nm, 150 nm, 100 nm, 75 nm, 50 nm, 45 nm or 40 nm to contact an inner surface of the DNA origami.

The DNA origami can change shape (conformation) in response to one or more certain environmental stimuli. Thus an area of the DNA origami can be an inner surface when the DNA origami takes on some conformations, but can be an outer surface when the device takes on other conformations. In some embodiments, the DNA origami can respond to certain environmental stimuli by taking on a new conformation.

In some embodiments, the staple strands of the DNA origami can be selected such that the DNA origami is substantially barrel- or tube-shaped. The staples of the DNA origami can be selected such that the barrel shape is closed at both ends or is open at one or both ends, thereby permitting particles to enter the interior of the barrel and access its inner surface. In certain embodiments, the barrel shape of the DNA origami can be a hexagonal tube.

In some embodiments, the staple strands of the DNA origami can be selected such that the DNA origami has a first domain and a second domain, wherein the first end of the first domain is attached to the first end of the second domain by one or more single-stranded DNA hinges, and the second end of the first domain is attached to the second domain of the second domain by the one or more molecular latches. The plurality of staples can be selected such that the second end of the first domain becomes unattached to the second end of the second domain if all of the molecular latches are contacted by their respective external stimuli. Latches can be formed from two or more staple stands, including at least one staple strand having at least one stimulus-binding domain that is able to bind to an external stimulus, such as a nucleic acid, a lipid or a protein, and at least one other staple strand having at least one latch domain that binds to the stimulus binding domain. The binding of the stimulus-binding domain to the latch domain supports the stability of a first conformation of the DNA origami.

Spatial labels can be delivered to a sample in three dimensions. For example a sample can be associated with an array, wherein the array has spatial labels distributed or distributable in three dimensions. A three dimensional array can be a scaffolding, a porous substrate, a gel, a series of channels, or the like.

A three dimensional pattern of spatial labels can be associated with a sample by injecting the samples into known locations with the sample, for example using a robot. A single needle can be used to serially inject spatial labels at different depths into a sample. An array of needles can inject spatial labels at different depths to generate a three dimensional distribution of labels.

In some embodiments, a three dimensional solid support can be a device. For example, a needle array device (e.g., a biopsy needle array device) can be a substrate. Stochastic barcodes of the disclosure can be attached to the device. Placing the device in and/or on a sample can bring the stochastic barcodes of the disclosure into proximity with targets in and/or on the sample. Different parts of the device can have stochastic barcodes with different spatial labels. For example, on a needle array device, each needle of the device can be coated with stochastic barcodes with different spatial labels on each needle. In this way, spatial labels can provide information about the location of the targets (e.g., location in orientation to the needle array).

### Probes

The solid support/substrate of the disclosure can comprise a plurality of probes. The probes can be, can be about, can be at least, or can be at least about, 1, 2, 3, 4, 5, 6, 7, 8, 9, 10, 11, 12, 13, 14, 15, 16, 17, 18, 19, or 20 or more nucleotides in length. The probes can be, can be at most, or can be at most about, 1, 2, 3, 4, 5, 6, 7, 8, 9, 10, 11, 12, 13, 14, 15, 16, 17, 18, 19, or 20 or more nucleotides in length.

The probes can be oligo(dT) probes. The probes can be any homopolymer sequence (e.g., poly(A), poly(C), poly(G), poly(U)).

The probes can be gene-specific. The probes can target any location of a gene (e.g., 3′ UTR, 5′ UTR, coding region, promoter). The probes on the substrate can be gene-specific for a plurality of genes. For example, a substrate can comprise probes that are gene-specific for, for about, for at least, or for at least about 10, 20, 30, 40, 50, 60, 70, 80, 90, 100, or a number or a range between any two of these values, genes. A substrate can comprise probes that are gene-specific for, for at most, or for at most about, 10, 20, 30, 40, 50, 60, 70, 80, 90, 100, or a number or a range between any two of these values, genes.

The plurality of gene-specific probes can be dispersed throughout the substrate evenly. The plurality of gene-specific probes can be dispersed throughout the substrate in discrete locations. There can be an equivalent number of gene-specific probes for each gene. There can be an inequivalent number of gene-specific probes for each gene. For examples, one or more gene-specific probes can be represented on the substrate at least or at least about 10%, 20%, 30%, 40%, 50%, 60%, 70%, 80%, or a number or a range between any two of these values, compared to one or more other gene-specific probes. One or more gene-specific probes can be represented on the substrate at most or at most about 10%, 20%, 30%, 40%, 50%, 60%, 70%, 80%, or a number or a range between any two of these values, compared to one or more other gene-specific probes.

The substrate can comprise a plurality of gene-specific probes for a plurality of genes and a plurality of oligo(dT) probes. The combination of gene-specific probes and oligo(dT) probes can be useful for bridge amplification methods of the disclosure. The ratio of a gene-specific probe to an oligo(dT) probe can be, can be about, can be at least, or can be at least about 1:1, 1:2, 1:3, 1:4, or 1:5 or more. The ratio of a gene-specific probe to an oligo(dT) probe can be, can be at most, or can be at most about, 1:1, 1:2, 1:3, 1:4, or 1:5 or more. The ratio of an oligo(dT) probe to a gene-specific probe can be, can be about, can be at least, or can be at least about, 1:1, 1:2, 1:3, 1:4, or 1:5 or more. The ratio of an oligo(dT) probe to a gene-specific probe can be at most or can be at most about 1:1, 1:2, 1:3, 1:4, or 1:5 or more.

The probes on the replicate substrate can comprise any of the probes, or combination of probes of the disclosure. The probes on the replicate substrate can be the same as the initial substrate. The probes on the replicate substrate can be different from the initial substrate. For example, the probes on the initial substrate can be gene-specific for a first location of a gene. The probes on the replicate slide can be gene-specific for a second location on the same gene. In this way, the probes can be used to identify (e.g., generate and/or detect) multiple amplicons from the same gene. The multiple amplicons can comprise different genetic features such as SNPs. Identification of multiple amplicons on the same gene can be useful for identification of SNPs and/or genetic mobility events (e.g., truncations, translocations, transpositions).

In some embodiments, the probes on the initial substrate can be oligo(dT) and the probes on the replicate substrate can be gene-specific or a combination of gene-specific and oligo(dT).

### Synthesis of Stochastic Barcodes on Solid Supports and Substrates

A stochastic barcode can be synthesized on a solid support (e.g., bead). Pre-synthesized stochastic barcodes (e.g., comprising the 5′amine that can link to the solid support) can be attached to solid supports (e.g., beads) through any of a variety of immobilization techniques involving functional group pairs on the solid support and the stochastic barcode. The stochastic barcode can comprise a functional group. The solid support (e.g., bead) can comprise a functional group. The stochastic barcode functional group and the solid support functional group can comprise, for example, biotin, streptavidin, primary amine(s), carboxyl(s), hydroxyl(s), aldehyde(s), ketone(s), and any combination thereof. A stochastic barcode can be tethered to a solid support, for example, by coupling (e.g. using 1-Ethyl-3-(3-dimethylaminopropyl) carbodiimide) a 5′ amino group on the stochastic barcode to the carboxyl group of the functionalized solid support. Residual non-coupled stochastic barcodes can be removed from the reaction mixture by performing multiple rinse steps. In some embodiments, the stochastic barcode and solid support are attached indirectly via linker molecules (e.g. short, functionalized hydrocarbon molecules or polyethylene oxide molecules) using similar attachment chemistries. The linkers can be cleavable linkers, e.g. acid-labile linkers or photo-cleavable linkers.

The stochastic barcodes can be synthesized on solid supports (e.g., beads) using any of a number of solid-phase oligonucleotide synthesis techniques, such as phosphodiester synthesis, phosphotriester synthesis, phosphite triester synthesis, and phosphoramidite synthesis. Single nucleotides can be coupled in step-wise fashion to the growing, tethered stochastic barcode. A short, pre-synthesized sequence (or block) of several oligonucleotides can be coupled to the growing, tethered stochastic barcode.

Stochastic barcodes can be synthesized by interspersing step-wise or block coupling reactions with one or more rounds of split-pool synthesis, in which the total pool of synthesis beads is divided into a number of individual smaller pools which are then each subjected to a different coupling reaction, followed by recombination and mixing of the individual pools to randomize the growing stochastic barcode sequence across the total pool of beads. Split-pool synthesis is an example of a combinatorial synthesis process in which a maximum number of chemical compounds are synthesized using a minimum number of chemical coupling steps. The potential diversity of the compound library thus created is determined by the number of unique building blocks (e.g. nucleotides) available for each coupling step, and the number of coupling steps used to create the library. For example, a split-pool synthesis comprising 10 rounds of coupling using 4 different nucleotides at each step will yield 4¹⁰=1,048,576 unique nucleotide sequences. In some embodiments, split-pool synthesis can be performed using enzymatic methods such as polymerase extension or ligation reactions rather than chemical coupling. For example, in each round of a split-pool polymerase extension reaction, the 3′ ends of the stochastic barcodes tethered to beads in a given pool can be hybridized with the 5′ends of a set of semi-random primers, e.g. primers having a structure of 5′-(M)ₖ-(X)ᵢ-(N)ⱼ-3′, where (X)ᵢ is a random sequence of nucleotides that is i nucleotides long (the set of primers comprising all possible combinations of (X)ᵢ), (N)ⱼ is a specific nucleotide (or series of j nucleotides), and (M)ₖ is a specific nucleotide (or series of k nucleotides), wherein a different deoxyribonucleotide triphosphate (dNTP) is added to each pool and incorporated into the tethered oligonucleotides by the polymerase.

The number of stochastic barcodes conjugated to or synthesized on a solid support can comprise at least 100, 1000, 10000, or 1000000 or more stochastic barcodes. The number of stochastic barcodes conjugated to or synthesized on a solid support can comprise at most 100, 1000, 10000, or 1000000 or more stochastic barcodes. The number of oligonucleotides conjugated to or synthesized on a solid support such as a bead can be at least 1-fold, 2-folds, 3-folds, 4-folds, 5-folds, 6-folds, 7-folds, 8-folds, 9-folds, or 10-folds more than the number of target nucleic acids in a cell. The number of oligonucleotides conjugated to or synthesized on a solid support such as a bead can be at most 1-fold, 2-folds, 3-folds, 4-folds, 5-folds, 6-folds, 7-folds, 8-folds, 9-folds, or 10-folds more than the number of target nucleic acids in a cell. At least 10, 20, 30, 40, 50, 60, 70, 80, 90 or 100% of the stochastic barcode can be bound by a target nucleic acid. At most 10, 20, 30, 40, 50, 60, 70, 80, 90 or 100% of the stochastic barcode can be bound by a target nucleic acid. At least 1, 2, 3, 4, 5, 6, 7, 8, 9, 10, 20, 30, 40, 50, 60, 70, 80, 90 or 100 or more different target nucleic acids can be captured by the stochastic barcode on the solid support. At most 1, 2, 3, 4, 5, 6, 7, 8, 9, 10, 20, 30, 40, 50, 60, 70, 80, 90 or 100 or more different target nucleic acids can be captured by the stochastic barcode on the solid support.

In some embodiments, stochastic barcodes can be synthesized by randomly distributing a single-stranded DNA mixture onto a substrate pre-coated with primers. The single-stranded DNA can hybridize to the primers. Bridge amplification can be performed to convert the single-stranded DNAs into a cluster. Sequencing can be performed to determine the sequence of the DNA at each cluster on the substrate. A sample can be applied to the substrate, followed by the stochastic barcoding methods of the disclosure.

In some embodiments, barcodes can be synthesized using size and/or electrophoretic mobility. For example, a mixture of stochastic barcodes can be prepared and separated into two-dimensions using gel electrophoresis. The gel can be the substrate.

### Methods of Stochastic Barcoding

The disclosure provides for methods for estimating the number of distinct targets at distinct locations in a physical sample (e.g., tissue, organ, tumor, cell). The methods can comprise placing the stochastic barcodes in close proximity with the sample, lysing the sample, associating distinct targets with the stochastic barcodes, amplifying the targets and/or digitally counting the targets. The method can further comprise analyzing and/or visualizing the information obtained from the spatial labels on the stochastic barcodes. In some embodiments, the methods comprise visualizing the plurality of targets in the sample. Mapping the plurality of targets onto the map of the sample can include generating a two dimensional map or a three dimensional map of the sample. The two dimensional map and the three dimensional map can be generated prior to or after stochastically barcoding the plurality of targets in the sample. Visualizing the plurality of targets in the sample can include mapping the plurality of targets onto a map of the sample. Mapping the plurality of targets onto the map of the sample can include generating a two dimensional map or a three dimensional map of the sample. The two dimensional map and the three dimensional map can be generated prior to or after stochastically barcoding the plurality of targets in the sample. in some embodiments, the two dimensional map and the three dimensional map can be generated before or after lysing the sample. Lysing the sample before or after generating the two dimensional map or the three dimensional map can include heating the sample, contacting the sample with a detergent, changing the pH of the sample, or any combination thereof.

### Contacting a Sample and a Stochastic Barcode

The disclosure provides for methods for contacting a sample (e.g., cells) to a substrate of the disclosure. A sample comprising, for example, a cell, organ, or tissue thin section, can be contacted to stochastic barcodes. The cells can be contacted, for example, by gravity flow wherein the cells can settle and create a monolayer. The sample can be a tissue thin section. The thin section can be placed on the substrate. The sample can be one-dimensional (e.g., form a planar surface). The sample (e.g., cells) can be spread across the substrate, for example, by growing/culturing the cells on the substrate.

When stochastic barcodes are in close proximity to targets, the targets can hybridize to the stochastic barcode. The stochastic barcodes can be contacted at a non-depletable ratio such that each distinct target can associate with a distinct stochastic barcode of the disclosure. To ensure efficient association between the target and the stochastic barcode, the targets can be crosslinked to the stochastic barcode.

In some embodiments, stochastically barcoding the one or more copies of a target chromosome (e.g., the first target chromosome) in the plurality of partitioned samples comprises hybridizing the first plurality of stochastic barcodes to the one or more copies of the first target chromosome. Stochastically barcoding the one or more copies of the first target chromosome in the plurality of partitioned samples can comprise generating one or more copies of a stochastically barcoded first target chromosome. Stochastically barcoding the one or more copies of the first target chromosome can comprise generating an indexed library of the stochastically barcoded first target chromosome.

### Cell Lysis

The location of the target chromosome(s) (e.g., the first target chromosome) can vary. The one or more copies of the target chromosome(s) can be inside one or more cells. In some embodiments, the one or more copies of the target chromosome(s) can be not inside any cell. The location of the target chromosome(s) can vary. The one or more copies of the target chromosome(s) can be inside one or more cells. In some embodiments, the one or more copies of the target chromosome(s) can be not inside any cell.

Prior to the distribution of chromosomes and stochastic barcodes, the cells can be lysed to liberate the target molecules. Cell lysis can be accomplished by any of a variety of means, for example, by chemical or biochemical means, by osmotic shock, or by means of thermal lysis, mechanical lysis, or optical lysis. Cells can be lysed by addition of a cell lysis buffer comprising a detergent (e.g. SDS, Li dodecyl sulfate, Triton X-100, Tween-20, or NP-40), an organic solvent (e.g. methanol or acetone), or digestive enzymes (e.g. proteinase K, pepsin, or trypsin), or any combination thereof. To increase the association of a target and a stochastic barcode, the rate of the diffusion of the target molecules can be altered by for example, reducing the temperature and/or increasing the viscosity of the lysate.

In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate.

In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1M or more Tris HCl. A lysis buffer can comprise at most about 0.01, 0.05, 0.1, 0.5, or 1M or more Tris HCL. A lysis buffer can comprise about 0.1M Tris HCl. The pH of the lysis buffer can be at least about 1, 2, 3, 4, 5, 6, 7, 8, 9, or 10 or more. The pH of the lysis buffer can be at most about 1, 2, 3, 4, 5, 6, 7, 8, 9, or 10 or more. In some embodiments, the pH of the lysis buffer is about 7.5. The lysis buffer can comprise a salt (e.g., LiCl). The concentration of salt in the lysis buffer can be at least about 0.1, 0.5, or 1M or more. The concentration of salt in the lysis buffer can be at most about 0.1, 0.5, or 1M or more. In some embodiments, the concentration of salt in the lysis buffer is about 0.5M. The lysis buffer can comprise a detergent (e.g., SDS, Li dodecyl sufate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7% or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7% or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCl, about pH 7.5, about 0.5M LiCl, about 1% lithium dodecyl sulfate, about 10 mM EDTA, and about 5 mM DTT.

ysis can be performed at a temperature of about 4, 10, 15, 20, 25, or 30 C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

### Attachment of Stochastic Barcodes to Target Nucleic Acid Molecules

Following lysis of the cells, release of nucleic acid molecules therefrom, and/or partitioning of the sample, the nucleic acid molecules can randomly associate with the stochastic barcodes of the co-localized solid support. Association can comprise hybridization of a stochastic barcode's target recognition region to a complementary portion of the target nucleic acid molecule (e.g., oligo(dT) of the stochastic barcode can interact with a poly(A) tail of a target). The assay conditions used for hybridization (e.g. buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of the cDNA molecule. For example, in a non-limiting example of stochastic barcoding illustrated in FIG. 2, at 216, double-stranded nucleotide fragmented can be denatured into single-stranded nucleotide fragments, and single-stranded nucleotide fragments can hybridize to stochastic barcodes on beads. For example, single-stranded nucleotide fragments can hybridize to the target-binding regions of stochastic barcodes.

Attachment can further comprise ligation of a stochastic barcode's target recognition region and a portion of the target nucleic acid molecule. For example, the target binding region can comprise a nucleic acid sequence that can be capable of specific hybridization to a restriction site overhang (e.g. an EcoRI sticky-end overhang). The assay procedure can further comprise treating the target nucleic acids with a restriction enzyme (e.g. EcoRI) to create a restriction site overhang. The stochastic barcode can then be ligated to any nucleic acid molecule comprising a sequence complementary to the restriction site overhang. A ligase (e.g., T4 DNA ligase) can be used to join the two fragments.

For example, in a non-limiting example of stochastic barcoding illustrated in FIG. 2, at 220, the labeled targets, for example labeled fragments from one of more target chromosomes (or a plurality of samples) (e.g., target-barcode molecules) can be subsequently pooled, for example, into a tube. The labeled targets from the plurality of target chromosomes can be pooled by, for example, retrieving the stochastic barcodes and/or the beads to which the target-barcode molecules are attached.

In some embodiments, the sample can comprise 24 target chromosomes, for example human chromosomes 1-22, X chromosome, and Y chromosome. For example, if each well contain at most one copy of one of the 24 target chromosomes, fragments from one copy of human chromosome 1 can be in a first microwell and can bind to a first bead. Fragments from a second copy of human chromosome 1 can be in a second microwell and bind to a second bead. Fragments from other copies of human chromosome 1 and human chromosomes other than human chromosome 1 can be in other microwells and bind to other beads. Consequently, fragments of copy 1 of human chromosome 1 can be in microwell_{chromosome 1, 1} and can bind to a bead_{chromosome 1, 1}; fragments of copy 2 of human chromosome 1 can be in microwell_{chromosome 1, 2} and can bind to a bead_{chromosome 1, 2}; . . . ; and fragments of copy N1 of human chromosome 1 can be in microwell_{chromosome 1, N1} and can bind to a bead_{chromosome 1, N1}. Similarly, fragments of copy 1 of human chromosome 2 can be in microwell_{chromosome 2, 1} and can bind to a bead_{chromosome 2, 1}; fragments of copy 2 of human chromosome 2 can be in microwell_{chromosome 2, 2} and can bind to a bead_{chromosome 2, 2}; . . . ; and fragments of copy N2 of human chromosome 1 can be in microwell_{chromosome 2, N2} and can bind to a bead_{chromosome 2, N2}. Similarly, fragments of copy 1 of human X chromosome can be in microwell_{chromosome X, 1} and can bind to a bead_{chromosome X, 1}; fragments of copy 2 of human X chromosome can be in microwell_{chromosome X, 2} and can bind to a bead_{chromosome X, 2}; . . . ; and fragments of copy NX of human X chromosome 1 can be in microwell_{chromosome X, NX} and can bind to a bead_{chromosome X, NX}. Similarly, fragments of copy 1 of human Y chromosome can be in microwell_{chromosome Y, 1} and can bind to a bead_{chromosome Y, 1}; fragments of copy 2 of human Y chromosome can be in microwell_{chromosome Y, 2} and can bind to a bead_{chromosome Y, 2}; . . . ; and fragments of copy NY of human Y chromosome 1 can be in microwell_{chromosome Y, NY} and can bind to a bead_{chromosome Y, NY}. For example, for a non-diseased male human subject and without any loss of target chromosome during sample collection and preparation, N1=N2= . . . =N22=2*NX=2*NY. For example, for a non-diseased female human subject and without any loss of target chromosome during sample collection and preparation, N1=N2= . . . =N22=NY. The bead_{chromosome 1, 1}, bead_{chromosome 1, 2}, . . . bead_{chromosome 1, N1}, bead_{chromosome 2, 1}, bead_{chromosome 2, 2}, . . . bead_{chromosome 1, N12}, . . . , bead_{chromosome X, 1}, bead_{chromosome X, 2}, . . . bead_{chromosome X, NX}, bead_{chromosome Y, 1}, bead_{chromosome Y, 1}, . . . bead_{chromosome 1, N1Y}, can be pooled, for example, into a tube.

The retrieval of solid support-based collections of attached target-barcode molecules can be implemented by use of magnetic beads and an externally-applied magnetic field. Once the target-barcode molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

### Reverse Transcription

The disclosure provides for a method to create a stochastic target-barcode conjugate using reverse transcription. The stochastic target-barcode conjugate can comprise the stochastic barcode and a complementary sequence of all or a portion of the target nucleic acid (i.e. a stochastically barcoded cDNA molecule). Reverse transcription of the associated RNA molecule can occur by the addition of a reverse transcription primer along with the reverse transcriptase. The reverse transcription primer can be an oligo(dT) primer, a random hexanucleotide primer, or a target-specific oligonucleotide primer. Oligo(dT) primers can be, or can be about, 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest.

In some embodiments, reverse transcription of the labeled-RNA molecule can occur by the addition of a reverse transcription primer. In some embodiments, the reverse transcription primer is an oligo(dT) primer, random hexanucleotide primer, or a target-specific oligonucleotide primer. Generally, oligo(dT) primers are 12-18 nucleotides in length and bind to the endogenous poly(A)+ tail at the 3′ end of mammalian mRNA. Random hexanucleotide primers can bind to mRNA at a variety of complementary sites. Target-specific oligonucleotide primers typically selectively prime the mRNA of interest.

Reverse transcription can occur repeatedly to produce multiple labeled-cDNA molecules. The methods disclosed herein can comprise conducting at least about 1, 2, 3, 4, 5, 6, 7, 8, 9, 10, 11, 12, 13, 14, 15, 16, 17, 18, 19, or 20 reverse transcription reactions. The method can comprise conducting at least about 25, 30, 35, 40, 45, 50, 55, 60, 65, 70, 75, 80, 85, 90, 95, or 100 reverse transcription reactions.

### DNA Synthesis

Disclosed herein are methods for creating stochastic target-barcode conjugates using DNA synthesis. The stochastic target-barcode conjugate can comprise the stochastic barcode and a complementary sequence of all or a portion of the target nucleic acid. DNA synthesis of the fragments of the one or more target chromosomes associated with beads (e.g., in 224 of FIG. 2) can occur by the addition of a primer along with the polymerase. The primer can be a random hexanucleotide primer, or a target-specific oligonucleotide primer. The primer can be the target-binding region. The primers can be, can be about, can be at least, or can be at most, 5, 6, 7, 8, 9, 10, 20, 30, 40, 50, 60, 70, 80, 90, 100, or a number or a range between any two of these values, nucleotides in length and bind to the fragments of the target chromosome. Random hexanucleotide primers can bind to fragments of the target chromosome at a variety of complementary sites. Target-specific oligonucleotide primers typically selectively prime the fragments of the target chromosomes that are of interest.

DNA synthesis can occur repeatedly to produce multiple labeled-fragments of the target chromosomes. The methods disclosed herein can comprise conducting about, at least, or at most 1, 2, 3, 4, 5, 6, 7, 8, 9, 10, 11, 12, 13, 14, 15, 16, 17, 18, 19, or 20 DNA synthesis reactions. The method can comprise conducting about, at least, or at most, 25, 30, 35, 40, 45, 50, 55, 60, 65, 70, 75, 80, 85, 90, 95, 100, or a number or a range between any two of these values, DNA synthesis reactions.

### Amplification

One or more nucleic acid amplification reactions (e.g., 228 of FIG. 2) can be performed to create multiple copies of the labeled target nucleic acid molecules, for example labeled fragments of one or more target chromosomes. Amplification can be performed in a multiplexed manner, wherein multiple target nucleic acid sequences are amplified simultaneously. The amplification reaction can be used to add sequencing adaptors to the nucleic acid molecules. The amplification reactions can comprise amplifying at least a portion of a sample label, if present. The amplification reactions can comprise amplifying at least a portion of the cellular and/or molecular label. The amplification reactions can comprise amplifying at least a portion of a sample tag, a chromosome label, a spatial label, a molecular label, a target nucleic acid, or a combination thereof. The amplification reactions can comprise amplifying 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a chromosome label, a spatial label, and/or a molecular label.

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the labeled nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a Qβ replicase (Qβ) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the labeled nucleic acid (e.g., labeled-RNA, labeled-DNA, labeled-cDNA) to produce a stochastically labeled-amplicon. The labeled-amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a chromosome label, and/or a molecular label. The stochastically labeled-amplicon can be a single-stranded molecule. The single-stranded molecule can comprise DNA, RNA, or a combination thereof. The nucleic acids of the disclosure can comprise synthetic or altered nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the plurality of stochastically labeled targets. The one or more primers can anneal to the 3′ end or 5′ end of the plurality of stochastically labeled targets. The one or more primers can anneal to an internal region of the plurality of stochastically labeled targets. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of stochastically labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a chromosome label, a molecular label, a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total stochastically labeled targets in one or more samples. The one or more primers can comprise at least 96 or more custom primers. The one or more primers can comprise at least 960 or more custom primers. The one or more primers can comprise at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 bp×2 sequencing can reveal the chromosome label and molecular index on read 1, the gene on read 2, and the sample index on index 1 read.

Amplification can be performed in one or more rounds. In some embodiments, there are multiple rounds of amplification. There can be two rounds of amplification. The first amplification can be an extension off X′ to generate the gene specific region. The second amplification can occur when a sample nucleic hybridizes to the X strand.

In some embodiments hybridization does not need to occur at the end of a nucleic acid molecule. In some embodiments a target nucleic acid within an intact strand of a longer nucleic acid is hybridized and amplified. For example a target within a longer section of genomic DNA or mRNA. Target can be more than 50 nt, more than 100 nt, or more that 1000 nt from an end of a polynucleotide.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonucelase digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

When the probes are gene-specific, the molecules can hybridize to the probes and be reverse transcribed and/or amplified. In some embodiments, after the nucleic acid has been synthesized (e.g., reverse transcribed), it can be amplified. Amplification can be performed in a multiplex manner, wherein multiple target nucleic acid sequences are amplified simultaneously. Amplification can add sequencing adaptors to the nucleic acid.

In some embodiments, amplification can be performed on the substrate, for example, with bridge amplification. cDNAs can be homopolymer tailed in order to generate a compatible end for bridge amplification using oligo(dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges.

The amplification reactions can comprise amplifying at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR real-time PCR, nested PCR, quantitative PCR multiplexed PCR, digital PCR, suppression PCR, semi-suppressive PCR and assembly PCR

In some embodiments, amplification of the labeled nucleic acids comprises non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), a Qβ replicase (Qβ), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or ramification extension amplification (RAM).

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least about 1, 2, 3, 4, 5, 6, 7, 8, 9, 10, 11, 12, 13, 14, 15, 16, 17, 18, 19, or 20 amplification reactions. Alternatively, the method comprises conducting at least about 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, stochastically barcoding the plurality of targets in the sample further comprises generating an indexed library of the stochastically barcoded fragments. The molecular labels of different stochastic barcodes can be different from one another. Generating an indexed library of the stochastically barcoded targets includes generating a plurality of indexed polynucleotides from the plurality of targets in the sample. For example, for an indexed library of the stochastically barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the stochastically barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the stochastically barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Stochastic barcoding can use nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adaptors can be added for sequencing using, for example, next generation sequencing (NGS). The sequencing results can be used to determine chromosome labels, molecular labels, and sequences of nucleotide fragments of the one or more copies of the one or more target chromosomes, for example at 232 of FIG. 2.

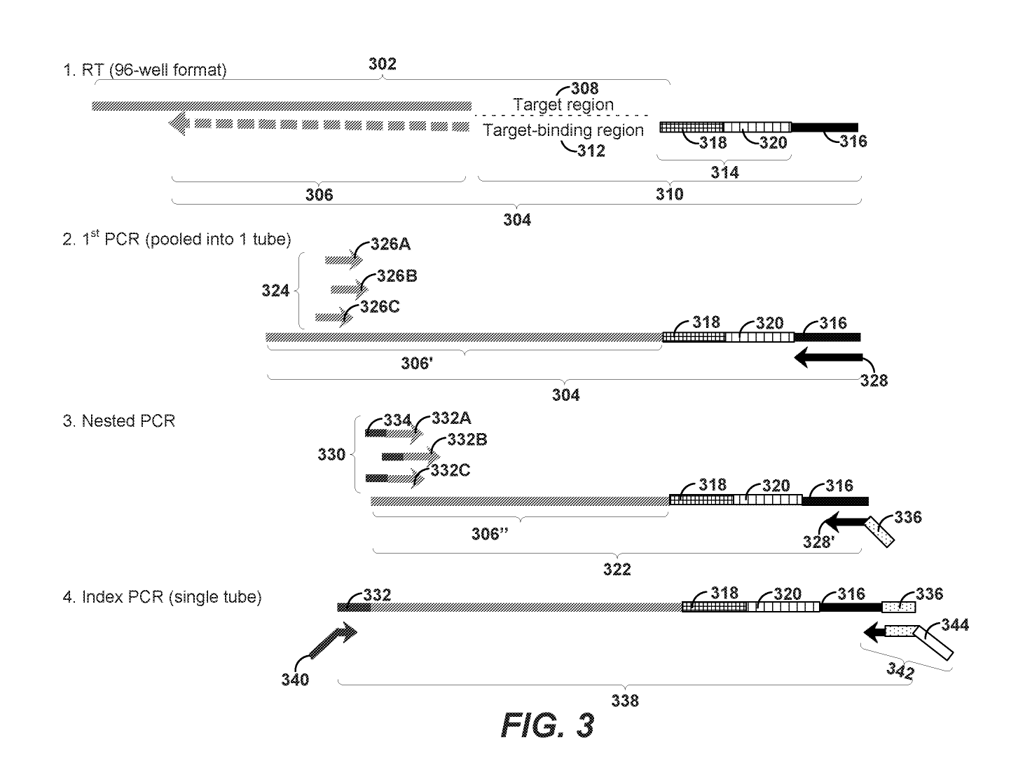

FIG. 3 is a schematic illustration showing a non-limiting exemplary process of generating an indexed library of the stochastically barcoded targets, for example fragments of chromosomes of interest. As shown in step 1, the DNA synthesis process can encode each fragment molecule with a unique molecular label, a chromosome label, and a universal PCR site. In particular, the fragment molecules 302 can be replicated to produce labeled fragment molecules 304, including a fragment portion 306, by the stochastic hybridization of a set of molecular identifier labels 310 to the target region 308 of the fragment molecules 302. Each of the molecular identifier labels 310 can comprise a target-binding region 312, a label region 314, and a universal PCR region 316.

In some embodiments, the chromosome label can include 3 to 20 nucleotides. In some embodiments, the molecular label can include 3 to 20 nucleotides. In some embodiments, each of the plurality of stochastic barcodes further comprises one or more of a universal label and a chromosome label, wherein universal labels are the same for the plurality of stochastic barcodes on the solid support and chromosome labels are the same for the plurality of stochastic barcodes on the solid support. In some embodiments, the universal label can include 3 to 20 nucleotides. In some embodiments, the chromosome label comprises 3 to 20 nucleotides.

In some embodiments, the label region 314 can include a molecular label 318 and a chromosome label 320. In some embodiments, the label region 314 can include one or more of a universal label, a dimension label, and a chromosome label. The molecular label 318 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The chromosome label 320 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The universal label can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. Universal labels can be the same for the plurality of stochastic barcodes on the solid support and chromosome labels are the same for the plurality of stochastic barcodes on the solid support. The dimension label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length.

In some embodiments, the label region 314 can comprise, comprise about, comprise at least, or comprise at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different labels, such as a molecular label 318 and a chromosome label 320. Each label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length. A set of molecular identifier labels 310 can contain, contain about, contain at least, or can be at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, molecular identifier labels 310. And the set of molecular identifier labels 310 can, for example, each contain a unique label region 314. The labeled fragment molecules 304 can be purified to remove excess molecular identifier labels 310. Purification can comprise Ampure bead purification.

As shown in step 2, products from the DNA synthesis process in step 1 can be pooled into 1 tube and PCR amplified with a 1^{st} PCR primer pool and a 1^{st} universal PCR primer. Pooling is possible because of the unique label region 314. In particular, the labeled fragment molecules 304 can be amplified to produce nested PCR labeled amplicons 322. Amplification can comprise multiplex PCR amplification. Amplification can comprise a multiplex PCR amplification with 96 multiplex primers in a single reaction volume. In some embodiments, multiplex PCR amplification can utilize, utilize about, utilize at least, or utilize at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, multiplex primers in a single reaction volume. Amplification can comprise 1^{st} PCR primer pool 324 of custom primers 326A-C targeting specific genes and a universal primer 328. The custom primers 326 can hybridize to a region within the fragment portion 306′ of the labeled fragment molecule 304. The universal primer 328 can hybridize to the universal PCR region 316 of the labeled fragment molecule 304.

As shown in step 3 of FIG. 3, products from PCR amplification in step 2 can be amplified with a nested PCR primers pool and a 2^{nd} universal PCR primer. Nested PCR can minimize PCR amplification bias. In particular, the nested PCR labeled amplicons 322 can be further amplified by nested PCR. The nested PCR can comprise multiplex PCR with nested PCR primers pool 330 of nested PCR primers 332A-C and a 2^{nd} universal PCR primer 328′ in a single reaction volume. The nested PCR primer pool 328 can contain, contain about, contain at least, or contain at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different nested PCR primers 330. The nested PCR primers 332 can contain an adaptor 334 and hybridize to a region within the fragment portion 306″ of the labeled amplicon 322. The universal primer 328′ can contain an adaptor 336 and hybridize to the universal PCR region 316 of the labeled amplicon 322. Thus, step 3 produces adaptor-labeled amplicon 338. In some embodiments, nested PCR primers 332 and the 2^{nd} universal PCR primer 328′ may not contain the adaptors 334 and 336. The adaptors 334 and 336 can instead be ligated to the products of nested PCR to produce adaptor-labeled amplicon 338.

As shown in step 4, PCR products from step 3 can be PCR amplified for sequencing using library amplification primers. In particular, the adaptors 334 and 336 can be used to conduct one or more additional assays on the adaptor-labeled amplicon 338. The adaptors 334 and 336 can be hybridized to primers 340 and 342. The one or more primers 340 and 342 can be PCR amplification primers. The one or more primers 340 and 342 can be sequencing primers. The one or more adaptors 334 and 336 can be used for further amplification of the adaptor-labeled amplicons 338. The one or more adaptors 334 and 336 can be used for sequencing the adaptor-labeled amplicon 338. The primer 342 can contain a plate index 344 so that amplicons generated using the same set of molecular identifier labels 318 can be sequenced in one sequencing reaction using next generation sequencing (NGS).

### Sequencing

Determining the number of different stochastically labeled nucleic acids can comprise determining the sequence of the labeled target, the spatial label, the molecular label, the sample label, and the chromosome label or any product thereof (e.g. labeled-amplicons, labeled-cDNA molecules, labeled fragment molecules). An amplified target can be subjected to sequencing. Determining the sequence of the stochastically labeled nucleic acid or any product thereof can comprise conducting a sequencing reaction to determine the sequence of at least a portion of a sample label, a spatial label, a chromosome label, a molecular label, at least a portion of the stochastically labeled target, a complement thereof, a reverse complement thereof, or any combination thereof.

Determination of the sequence of a nucleic acid (e.g. amplified nucleic acid, labeled nucleic acid, cDNA copy of a labeled nucleic acid, etc.) can be performed using variety of sequencing methods including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads, wobble sequencing, multiplex sequencing, polymerized colony (POLONY) sequencing; nanogrid rolling circle sequencing (ROLONY), allele-specific oligo ligation assays (e.g., oligo ligation assay (OLA), single template molecule OLA using a ligated linear probe and a rolling circle amplification (RCA) readout, ligated padlock probes, or single template molecule OLA using a ligated circular padlock probe and a rolling circle amplification (RCA) readout), and the like.

In some embodiments, determining the sequence of the labeled nucleic acid or any product thereof comprises paired-end sequencing, nanopore sequencing, high-throughput sequencing, shotgun sequencing, dye-terminator sequencing, multiple-primer DNA sequencing, primer walking, Sanger dideoxy sequencing, Maxim-Gilbert sequencing, pyrosequencing, true single molecule sequencing, or any combination thereof. Alternatively, the sequence of the labeled nucleic acid or any product thereof can be determined by electron microscopy or a chemical-sensitive field effect transistor (chemFET) array.

High-throughput sequencing methods, such as cyclic array sequencing using platforms such as Roche 454, Illumina Solexa, ABI-SOLiD, ION Torrent, Complete Genomics, Pacific Bioscience, Helicos, or the Polonator platform, can also be utilized. In some embodiment, sequencing can comprise MiSeq sequencing. In some embodiment, sequencing can comprise HiSeq sequencing.

The stochastically labeled targets can comprise nucleic acids representing from about 0.01% of the genes of an organism's genome to about 100% of the genes of an organism's genome. For example, about 0.01% of the genes of an organism's genome to about 100% of the genes of an organism's genome can be sequenced using a target complimentary region comprising a plurality of multimers by capturing the genes containing a complimentary sequence from the sample. In some embodiments, the labeled nucleic acids comprise nucleic acids representing from about 0.01% of the transcripts of an organism's transcriptome to about 100% of the transcripts of an organism's transcriptome. For example, about 0.501% of the transcripts of an organism's transcriptome to about 100% of the transcripts of an organism's transcriptome can be sequenced using a target complimentary region comprising a poly(T) tail by capturing the mRNAs from the sample.

Determining the sequences of the spatial labels and the molecular labels of the plurality of the stochastic barcodes can include sequencing 0.00001%, 0.0001%, 0.001%, 0.01%, 0.1%, 1%, 2%, 3%, 4%, 5%, 6%, 7%, 8%, 9%, 10%, 20%, 30%, 40%, 50%, 60%, 70%, 80%, 90%, 99%, 100%, or a number or a range between any two of these values, of the plurality of stochastic barcodes. Determining the sequences of the labels of the plurality of stochastic barcodes, for example the cellular labels, the spatial labels, and the molecular labels, can include sequencing 1, 10, 20, 30, 40, 50, 60, 70, 80, 90, 100, 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or a number or a range between any two of these values, of the plurality of stochastic barcodes. Sequencing some or all of the plurality of stochastic barcodes can include generating sequences with read lengths of, of about, of at least, or of at most, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or a number or a range between any two of these values, of nucleotides or bases.

Sequencing can comprise sequencing at least or at least about 10, 20, 30, 40, 50, 60, 70, 80, 90, 100 or more nucleotides or base pairs of the labeled nucleic acid. Sequencing can comprise sequencing at least or at least about 200, 300, 400, 500, 600, 700, 800, 900, 1,000 or more nucleotides or base pairs of the labeled nucleic acid. Sequencing can comprise sequencing at least or at least about 1500, 2000, 3000, 4000, 5000, 6000, 7000, 8000, 9000, or 10000 or more nucleotides or base pairs of the labeled nucleic acid.

Sequencing can comprise at least about 200, 300, 400, 500, 600, 700, 800, 900, 1,000 or more sequencing reads per run. In some embodiments, sequencing comprises sequencing at least or at least about 1500, 2000, 3000, 4000, 5000, 6000, 7000, 8000, 9000, or 10000 or more sequencing reads per run. Sequencing can comprise less than or equal to about 1,600,000,000 sequencing reads per run. Sequencing can comprise less than or equal to about 200,000,000 reads per run.

### Samples

In some embodiments, the one or more copies of the target chromosome (e.g., the first target chromosome) comprise chromosomes from fetal cells. In some embodiments, the one or more copies of the target chromosome (e.g., the first target chromosome) comprise chromosome fragments from a biological sample (e.g., blood) of a pregnant woman. In some embodiments, the one or more copies of the first target chromosome comprise chromosomes from cancer cells. The first target chromosome can be a human chromosome.

A sample for use in the method of the disclosure can comprise one or more cells. A sample can refer to one or more cells. In some embodiments, the plurality of cells can include one or more cell types. At least one of the one or more cell types can be brain cell, heart cell, cancer cell, circulating tumor cell, organ cell, epithelial cell, metastatic cell, benign cell, primary cell, circulatory cell, or any combination thereof. In some embodiments, the cells are cancer cells excised from a cancerous tissue, for example, breast cancer, lung cancer, colon cancer, prostate cancer, ovarian cancer, pancreatic cancer, brain cancer, melanoma and non-melanoma skin cancers, and the like. In some embodiments, the cells are derived from a cancer but collected from a bodily fluid (e.g. circulating tumor cells). Non-limiting examples of cancers can include, adenoma, adenocarcinoma, squamous cell carcinoma, basal cell carcinoma, small cell carcinoma, large cell undifferentiated carcinoma, chondrosarcoma, and fibrosarcoma. The sample can include a tissue, a cell monolayer, fixed cells, a tissue section, or any combination thereof. The sample can include a biological sample, a clinical sample, an environmental sample, a biological fluid, a tissue, or a cell from a subject. The sample can be obtained from a human, a mammal, a dog, a rat, a mouse, a fish, a fly, a worm, a plant, a fungus, a bacterium, a virus, a vertebrate, or an invertebrate.

In some embodiments, the cells are cells that have been infected with virus and contain viral oligonucleotides. In some embodiments, the viral infection can be caused by a virus selected from the group consisting of double-stranded DNA viruses (e.g. adenoviruses, herpes viruses, pox viruses), single-stranded (+ strand or ““sense””) DNA viruses (e.g. parvoviruses), double-stranded RNA viruses (e.g. reoviruses), single-stranded (+ strand or sense) RNA viruses (e.g. picornaviruses, togaviruses), single-stranded (− strand or antisense) RNA viruses (e.g. orthomyxoviruses, rhabdoviruses), single-stranded ((+ strand or sense) RNA viruses with a DNA intermediate in their life-cycle) RNA-RT viruses (e.g. retroviruses), and double-stranded DNA-RT viruses (e.g. hepadnaviruses). Exemplary viruses can include, but are not limited to, SARS, HIV, coronaviruses, Ebola, Malaria, Dengue, Hepatitis C, Hepatitis B, and Influenza.

In some embodiments, the cells are bacteria. These can include either gram-positive or gram-negative bacteria. Examples of bacteria that can be analyzed using the disclosed methods, devices, and systems include, but are not limited to, Actinomedurae, Actinomyces israelii, Bacillus anthracis. Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae and the like. Gram negative bacteria include, but are not limited to, Afipia felis, Bacteroides, Bartonella bacilliformis, Bortadella pertussis, Borrelia burgdorferi, Borrelia recurrentis. Brucella, Calymmatobacterium granulomatis, Campylobacter, Escherichia coli, Francisella tularensis, Gardnerella vaginalis, Haemophilius aegyptius, Haemophilius ducreyi, Haemophilius influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum. Vibrio cholerae, Yersinia enterocolitica, Yersinia pestis and the like. Other bacteria can include Myobacterium avium, Myobacterium leprae, Myobacterium tuberculosis, Bartonella henseiae, Chlamydia psittaci, Chlamydia trachomatis, Coxiella bumetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci and the like.

In some embodiments, the cells are fungi. Non-limiting examples of fungi that can be analyzed using the disclosed methods, devices, and systems include, but are not limited to, Aspergilli, Candidac, Candida albicans, Coccidioides immitis, Cryptococci, and combinations thereof.

In some embodiments, the cells are protozoans or other parasites. Examples of parasites to be analyzed using the methods, devices, and systems of the present disclosure include, but are not limited to, Balantidium coli, Cryptosporidium parvum, Cyclospora cayatanensis, Encephalitozoa, Entamoeba histolytica, Enterocytozoon bieneusi, Giardia lamblia, Leishmaniae, Plasmodii. Toxoplasma gondii, Trypanosomae, trapezoidal amoeba, worms (e.g., helminthes), particularly parasitic worms including, but not limited to, Nematoda (roundworms. e.g., whipworms, hookworms, pinworms, ascarids, filarids and the like), Cestoda (e.g., tapeworms).

As used herein, the term ““cell”” can refer to one or more cells. In some embodiments, the cells are normal cells, for example, human cells in different stages of development, or human cells from different organs or tissue types (e.g. white blood cells, red blood cells, platelets, epithelial cells, endothelial cells, neurons, glial cells, fibroblasts, skeletal muscle cells, smooth muscle cells, gametes, or cells from the heart, lungs, brain, liver, kidney, spleen, pancreas, thymus, bladder, stomach, colon, small intestine). In some embodiments, the cells can be undifferentiated human stem cells, or human stem cells that have been induced to differentiate. In some embodiments, the cells can be fetal human cells. The fetal human cells can be obtained from a mother pregnant with the fetus. In some embodiments, the cells are rare cells. A rare cell can be, for example, a circulating tumor cell (CTC), circulating epithelial cell, circulating endothelial cell, circulating endometrial cell, circulating stem cell, stem cell, undifferentiated stem cell, cancer stem cell, bone marrow cell, progenitor cell, foam cell, mesenchymal cell, trophoblast, immune system cell (host or graft), cellular fragment, cellular organelle (e.g. mitochondria or nuclei), pathogen infected cell, and the like.

In some embodiments, the cells are non-human cells, for example, other types of mammalian cells (e.g. mouse, rat, pig, dog, cow, or horse). In some embodiments, the cells are other types of animal or plant cells. In other embodiments, the cells can be any prokaryotic or eukaryotic cells.

In some embodiments, a first cell sample is obtained from a person not having a disease or condition, and a second cell sample is obtained from a person having the disease or condition. In some embodiments, the persons are different. In some embodiments, the persons are the same but cell samples are taken at different time points. In some embodiments, the persons are patients, and the cell samples are patient samples. The disease or condition can be a cancer, a bacterial infection, a viral infection, an inflammatory disease, a neurodegenerative disease, a fungal disease, a parasitic disease, a genetic disorder, or any combination thereof.

In some embodiments, cells suitable for use in the presently disclosed methods can range in size from about 2 micrometers to about 100 micrometers in diameter. In some embodiments, the cells can have diameters of, of about, of at least, or of at least about, 2, 5, 10, 15, 20, 30, 40, 50, 60, 70, 80, 90, 100 micrometers, or a number or a range between any two of these values. In some embodiments, the cells can have diameters of, of at most, or of at most about, 100, 90, 80, 70, 60, 50, 40, 30, 20, 15, 10, 5, 2 micrometers, or a number or a range between any two of these values. The cells can have a diameter of any value within a range, for example from about 5 micrometers to about 85 micrometers. In some embodiments, the cells have diameters of about 10 micrometers.

In some embodiments the cells are sorted prior to associating a cell with a bead. For example the cells can be sorted by fluorescence-activated cell sorting or magnetic-activated cell sorting, or more generally by flow cytometry. The cells can be filtered by size. In some embodiments a retentate contains the cells to be associated with the bead. In some embodiments the flow through contains the cells to be associated with the bead.

A sample can refer to a plurality of cells. The sample can refer to a monolayer of cells. The sample can refer to a thin section (e.g., tissue thin section). The sample can refer to a solid or semi-solid collection of cells that can be place in one dimension on an array.

### Diffusion Across a Substrate

When a sample (e.g., cell) is stochastically barcoded according to the methods of the disclosure, the cell can be lysed. In some embodiments, lysis of a cell can result in the diffusion of the contents of the lysis (e.g., cell contents) away from the initial location of lysis. In other words, the lysis contents can move into a larger surface area than the surface area taken up by the cell.

Diffusion of sample lysis mixture (e.g., comprising targets) can be modulated by various parameters including, but not limited to, viscosity of the lysis mixture, temperature of the lysis mixture, the size of the targets, the size of physical barriers in a substrate, the concentration of the lysis mixture, and the like. For example, the temperature of the lysis reaction can be performed at a temperature of at least 1, 2, 3, 4, 5, 10, 15, 20, 25, 30, 35, or 40° C. or more. The temperature of the lysis reaction can be performed at a temperature of at most 1, 2, 3, 4, 5, 10, 15, 20, 25, 30, 35, or 40° C. or more. The viscosity of the lysis mixture can be altered by, for example, adding thickening reagents (e.g., glycerol, beads) to slow the rate of diffusion. The viscosity of the lysis mixture can be altered by, for example, adding thinning reagents (e.g., water) to increase the rate of diffusion. A substrate can comprise physical barriers (e.g., wells, microwells, microhills) that can alter the rate of diffusion of targets from a sample. The concentration of the lysis mixture can be altered to increase or decrease the rate of diffusion of targets from a sample. The concentration of a lysis mixture can be increased or decreased by at least 1, 2, 3, 4, 5, 6, 7, 8, or 9 or more fold. The concentration of a lysis mixture can be increased or decreased by at most 1, 2, 3, 4, 5, 6, 7, 8, or 9 or more fold.

The rate of diffusion can be increased. The rate of diffusion can be decreased. The rate of diffusion of a lysis mixture can be increased or decreased by at least 1, 2, 3, 4, 5, 6, 7, 8, 9, or 10 or more fold compared to an un-altered lysis mixture. The rate of diffusion of a lysis mixture can be increased or decreased by at most 1, 2, 3, 4, 5, 6, 7, 8, 9, or 10 or more fold compared to an un-altered lysis mixture. The rate of diffusion of a lysis mixture can be increased or decreased by at least 10, 20, 30, 40, 50, 60, 70, 80, 90 or 100% compared to an un-altered lysis mixture. The rate of diffusion of a lysis mixture can be increased or decreased by at least 10, 20, 30, 40, 50, 60, 70, 80, 90 or 100% compared to an un-altered lysis mixture.

### Data Analysis and Display Software

Sequencing the molecularly indexed polynucleotide library can, in some embodiments, include deconvoluting the sequencing result from sequencing the library, using, for example, a software-as-a-service platform.

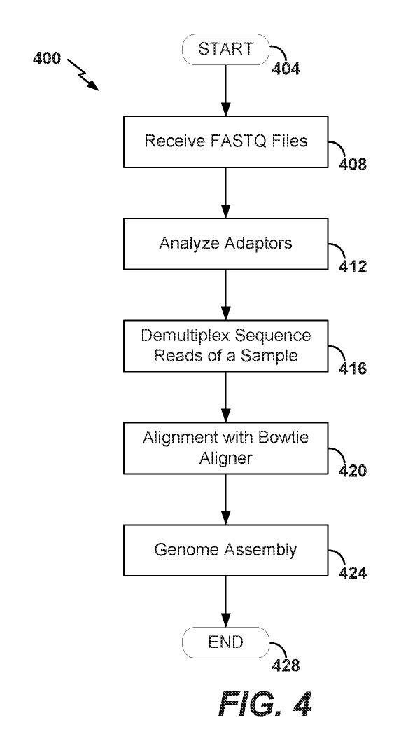

FIG. 4 is a flowchart showing non-limiting exemplary steps of data analysis 400 for use, for example, at 124B of FIG. 1B. Data analysis can be provided in a secure online cloud environment. In some embodiments, data analysis can be performed using a software-as-a-service platform. Non-limiting examples of secure online cloud environments include the Seven Bridges Genomics platform. The Seven Bridges Genomics platform is a non-limiting example of a software-as-a-service platform.

As shown in FIG. 4, data analysis 400 starts at 404. At 408, a sequencing result is received from, the sequencing of the indexed library. Non-limiting examples of the formats of the sequencing result received include EMBL, FASTA, and FASTQ format. The sequencing result can include sequence reads of a molecularly indexed polynucleotide library. The molecularly indexed polynucleotide library can include sequence information of a plurality of single cells. Sequence information of multiple single cells can be deconvoluted by the following steps. At 412 the sequences of the adaptors used for sequencing at 122B are determined, analyzed, and discarded for subsequent analysis. The one or more adaptors can include the adaptor 334 and 336 in FIG. 3.

At 416, the sequencing result of a molecularly indexed polynucleotide library is demultiplexed. Demultiplexing can include classifying the sequence reads as belonging to one of a plurality of single cells. Classifying the sequence reads as belonging to one of a plurality of single cells can be based on the label region 314, for example the sample label 320. The sequence reads belonging to one fragment molecule can be distinguished from those belonging to another fragment molecule based on the label region 314, for example the molecular label 318. At 420, sequence reads can be aligned to genome sequences using an aligner. Non-limiting examples of the aligner used at 420 include the Bowtie aligner, ClustalW, BLAST, ExPASy, and T-COFFEE. At 424, the genome sequence is reconstructed from the fragment sequences that are uniquely identified by the label region 314. The output of data analysis 400 can include a spreadsheet of read alignment and genome sequence. Data analysis 400 ends at 428.

### Data Analysis and Visualization of Spatial Resolution of Targets

The disclosure provides for methods for estimating the number and position of targets with stochastic barcoding and digital counting using spatial labels. The data obtained from the methods of the disclosure can be visualized on a map. A map of the number and location of targets from a sample can be constructed using information generated using the methods described herein. The map can be used to locate a physical location of a target. The map can be used to identify the location of multiple targets. The multiple targets can be the same species of target, or the multiple targets can be multiple different targets. For example a map of a brain can be constructed to show the digital count and location of multiple targets.

The map can be generated from data from a single sample. The map can be constructed using data from multiple samples, thereby generating a combined map. The map can be constructed with data from tens, hundreds, and/or thousands of samples. A map constructed from multiple samples can show a distribution of digital counts of targets associated with regions common to the multiple samples. For example, replicated assays can be displayed on the same map. At least 1, 2, 3, 4, 5, 6, 7, 8, 9, or 10 or more replicates can be displayed (e.g., overlaid) on the same map. At most 1, 2, 3, 4, 5, 6, 7, 8, 9, or 10 or more replicates can be displayed (e.g., overlaid) on the same map. The spatial distribution and number of targets can be represented by a variety of statistics.

Combining data from multiple samples can increase the locational resolution of the combined map. The orientation of multiple samples can be registered by common landmarks, wherein the individual locational measurements across samples are at least in part non-contiguous. A particular example is sectioning a sample using a microtome on one axis and then sectioning a second sample along a different access. The combined dataset will give three dimensional spatial locations associated with digital counts of targets. Multiplexing the above approach will allow for high resolution three dimensional maps of digital counting statistics.

In some embodiments of the instrument system, the system will comprise computer-readable media that includes code for providing data analysis for the sequence datasets generated by performing single cell, stochastic barcoding assays. Examples of data analysis functionality that can be provided by the data analysis software include, but are not limited to, (i) algorithms for decoding/demultiplexing of the sample label, chromosome label, spatial label, and molecular label, and target sequence data provided by sequencing the stochastic barcode library created in running the assay, (ii) algorithms for determining the number of reads per gene per cell, and the number of unique transcript molecules per gene per cell, based on the data, and creating summary tables, (iii) statistical analysis of the sequence data. e.g. for clustering of cells by gene expression data, or for predicting confidence intervals for determinations of the number of transcript molecules per gene per cell, etc., (iv) algorithms for identifying sub-populations of rare cells, for example, using principal component analysis, hierarchical clustering, k-mean clustering, self-organizing maps, neural networks etc., (v) sequence alignment capabilities for alignment of gene sequence data with known reference sequences and detection of mutation, polymorphic markers and splice variants, and (vi) automated clustering of molecular labels to compensate for amplification or sequencing errors. In some embodiments, commercially-available software can be used to perform all or a portion of the data analysis, for example, the Seven Bridges (https://www.sbgenomics.com/) software can be used to compile tables of the number of copies of one or more genes occurring in each cell for the entire collection of cells. In some embodiments, the data analysis software can include options for outputting the sequencing results in useful graphical formats, e.g. heatmaps that indicate the number of copies of one or more genes occurring in each cell of a collection of cells. In some embodiments, the data analysis software can further comprise algorithms for extracting biological meaning from the sequencing results, for example, by correlating the number of copies of one or more genes occurring in each cell of a collection of cells with a type of cell, a type of rare cell, or a cell derived from a subject having a specific disease or condition. In some embodiment, the data analysis software can further comprise algorithms for comparing populations of cells across different biological samples.

In some embodiments all of the data analysis functionality can be packaged within a single software package. In some embodiments, the complete set of data analysis capabilities can comprise a suite of software packages. In some embodiments, the data analysis software can be a standalone package that is made available to users independently of the assay instrument system. In some embodiments, the software can be web-based, and can allow users to share data.

In some embodiments all of the data analysis functionality can be packaged within a single software package. In some embodiments, the complete set of data analysis capabilities can comprise a suite of software packages. In some embodiments, the data analysis software can be a standalone package that is made available to users independently of the assay instrument system. In some embodiments, the software can be web-based, and can allow users to share data.

### System Processors and Networks

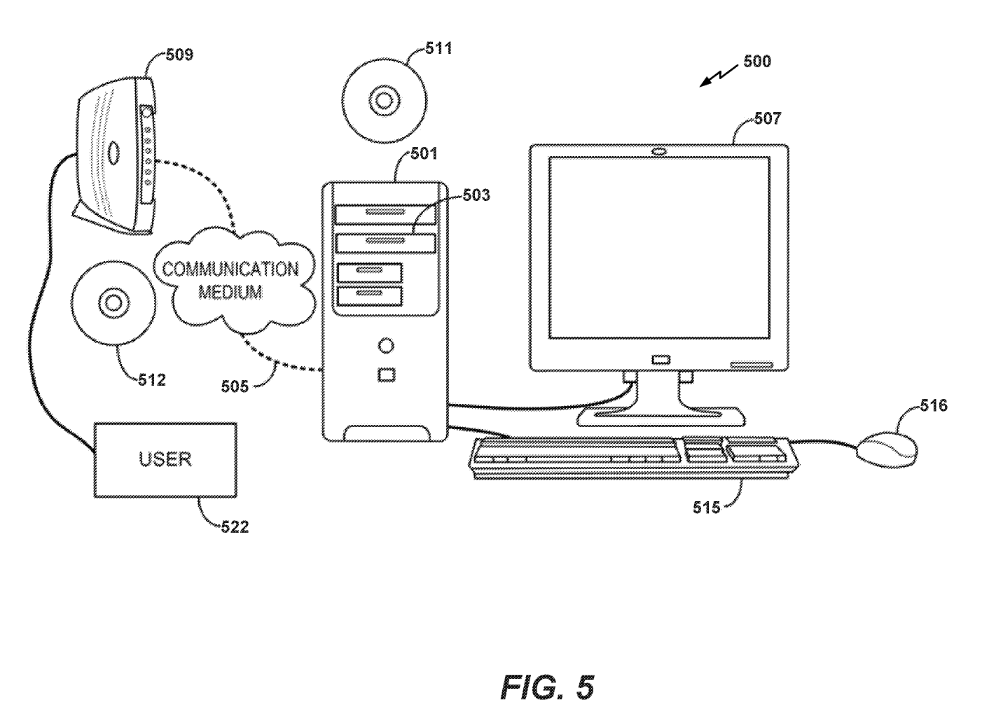

In general, the computer or processor included in the presently disclosed instrument systems, as illustrated in FIG. 5, can be further understood as a logical apparatus that can read instructions from media 511 or a network port 505, which can optionally be connected to server 509 having fixed media 512. The system 500, such as shown in FIG. 5 can include a CPU 501, disk drives 503, optional input devices such as keyboard 515 or mouse 516 and optional monitor 507. Data communication can be achieved through the indicated communication medium to a server at a local or a remote location. The communication medium can include any means of transmitting or receiving data. For example, the communication medium can be a network connection, a wireless connection or an internet connection. Such a connection can provide for communication over the World Wide Web. It is envisioned that data relating to the present disclosure can be transmitted over such networks or connections for reception or review by a party 522 as illustrated in FIG. 5.

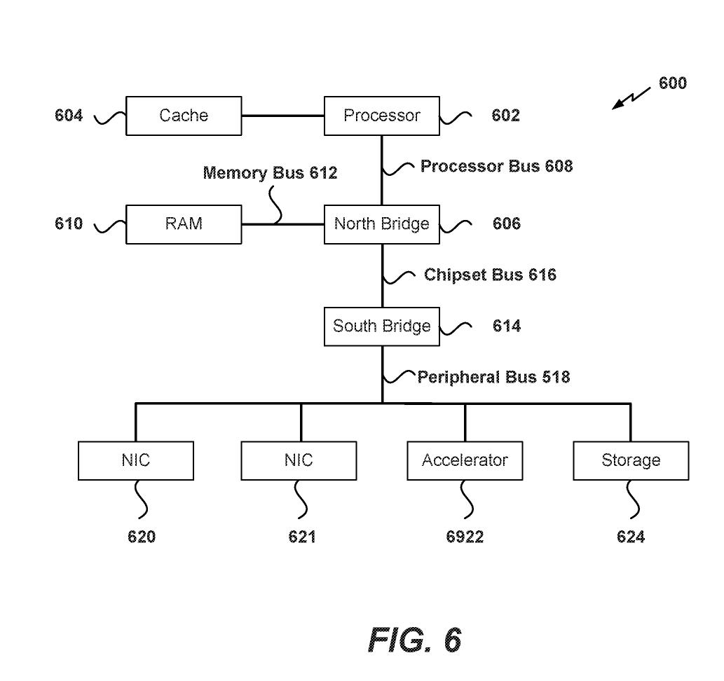

FIG. 6 illustrates an exemplary embodiment of a first example architecture of a computer system 600 that can be used in connection with example embodiments of the present disclosure. As depicted in FIG. 6, the example computer system can include a processor 602 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some embodiments, multiple processors or processors with multiple cores can also be used, whether in a single computer system, in a cluster, or distributed across systems over a network comprising a plurality of computers, cell phones, or personal data assistant devices.

As illustrated in FIG. 6, a high speed cache 604 can be connected to, or incorporated in, the processor 602 to provide a high speed memory for instructions or data that have been recently, or are frequently, used by processor 602. The processor 602 is connected to a north bridge 606 by a processor bus 608. The north bridge 606 is connected to random access memory (RAM) 610 by a memory bus 612 and manages access to the RAM 610 by the processor 602. The north bridge 606 is also connected to a south bridge 614 by a chipset bus 616. The south bridge 614 is, in turn, connected to a peripheral bus 618. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 118. In some alternative architectures, the functionality of the north bridge can be incorporated into the processor instead of using a separate north bridge chip.

In some embodiments, system 600 can include an accelerator card 622 attached to the peripheral bus 618. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 624 and can be loaded into RAM 610 or cache 604 for use by the processor. The system 600 includes an operating system for managing system resources; non-limiting examples of operating systems include: Linux. Windows™, MACOS™, BlackBerry OS™, iOS™, and other functionally-equivalent operating systems, as well as application software running on top of the operating system for managing data storage and optimization in accordance with example embodiments of the present invention.

In this example, system 600 also includes network interface cards (NICs) 620 and 621 connected to the peripheral bus for providing network interfaces to external storage, such as Network Attached Storage (NAS) and other computer systems that can be used for distributed parallel processing.

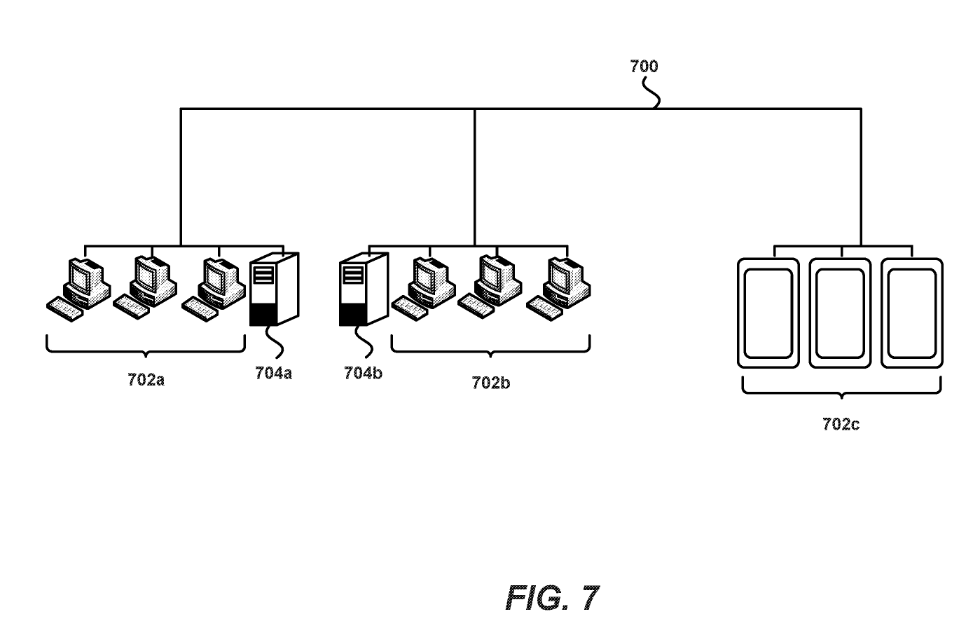

FIG. 7 illustrates an exemplary diagram showing a network 700 with a plurality of computer systems 702a, and 702b, a plurality of cell phones and personal data assistants 702c, and Network Attached Storage (NAS) 704a, and 704b. In example embodiments, systems 712a, 712b, and 712c can manage data storage and optimize data access for data stored in Network Attached Storage (NAS) 714a and 714b. A mathematical model can be used for the data and be evaluated using distributed parallel processing across computer systems 712a, and 712b, and cell phone and personal data assistant systems 712c. Computer systems 712a, and 712b, and cell phone and personal data assistant systems 712c can also provide parallel processing for adaptive data restructuring of the data stored in Network Attached Storage (NAS) 714a and 714b. FIG. 7 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

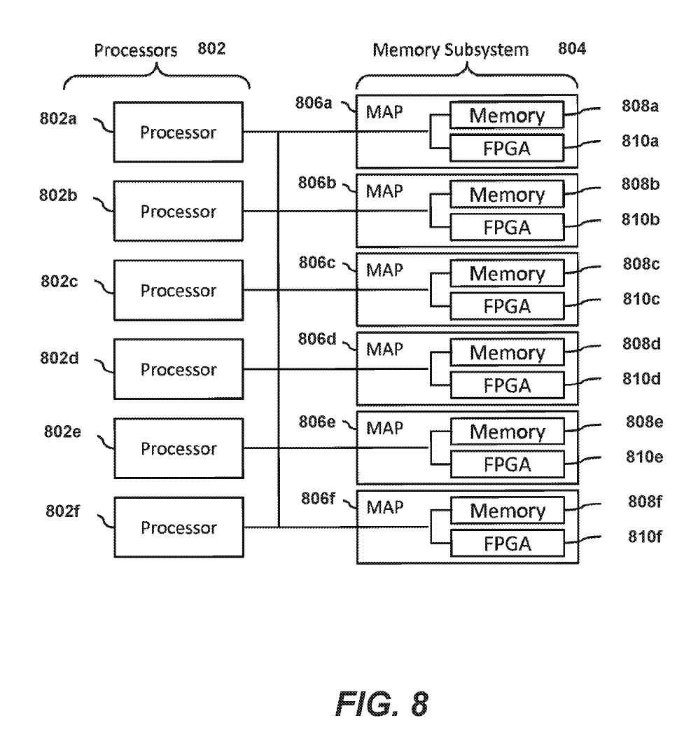

FIG. 8 illustrates an exemplary a block diagram of a multiprocessor computer system 800 using a shared virtual address memory space in accordance with an example embodiment. The system includes a plurality of processors 802a-f that can access a shared memory subsystem 804. The system incorporates a plurality of programmable hardware memory algorithm processors (MAPs) 806a-f in the memory subsystem 804. Each MAP 806a-f can comprise a memory 808a-f and one or more field programmable gate arrays (FPGAs) 810a-f. The MAP provides a configurable functional unit and particular algorithms or portions of algorithms can be provided to the FPGAs 810a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 808a-f, allowing it to execute tasks independently of, and asynchronously from, the respective microprocessor 802a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer subsystem of the present disclosure can be implemented using software modules executing on any of the above or other computer architectures and systems. In other embodiments, the functions of the system can be implemented partially or completely in firmware, programmable logic devices such as field programmable gate arrays (FPGAs), system on chips (SOLs), application specific integrated circuits (ASICs), or other processing and logic elements. For example, the Set Processor and Optimizer can be implemented with hardware acceleration through the use of a hardware accelerator card, such as accelerator card.

### Kits

Disclosed herein are kits for performing single cell, stochastic barcoding assays. The kit can comprise one or more substrates (e.g., microwell array), either as a free-standing substrate (or chip) comprising one or more microwell arrays, or packaged within one or more flow-cells or cartridges, and one or more solid support suspensions, wherein the individual solid supports within a suspension comprise a plurality of attached stochastic barcodes of the disclosure. In some embodiments, the kit can further comprise a mechanical fixture for mounting a free-standing substrate in order to create reaction wells that facilitate the pipetting of samples and reagents into the substrate. The kit can further comprise reagents, e.g. lysis buffers, rinse buffers, or hybridization buffers, for performing the stochastic barcoding assay. The kit can further comprise reagents (e.g. enzymes, primers, dNTPs, NTPs, RNAse inhibitors or buffers) for performing nucleic acid extension reactions, for example, reverse transcription reactions. The kit can further comprise reagents (e.g. enzymes, universal primers, sequencing primers, target-specific primers, or buffers) for performing amplification reactions to prepare sequencing libraries. The kit can comprise reagents for performing the label lithography method of the disclosure (e.g., pre-spatial labels and reagents for activating the activatable consensus sequence).

The kit can comprise one or more molds, for example, molds comprising an array of micropillars, for casting substrates (e.g., microwell arrays), and one or more solid supports (e.g., bead), wherein the individual beads within a suspension comprise a plurality of attached stochastic barcodes of the disclosure. The kit can further comprise a material for use in casting substrates (e.g. agarose, a hydrogel, PDMS, and the like).

The kit can comprise one or more substrates that are pre-loaded with solid supports comprising a plurality of attached stochastic barcodes of the disclosure. In some embodiments, there can be on solid support per microwell of the substrate. In some embodiments, the plurality of stochastic barcodes can be attached directly to a surface of the substrate, rather than to a solid support. In any of these embodiments, the one or more microwell arrays can be provided in the form of free-standing substrates (or chips), or they can be packed in flow-cells or cartridges.

In some embodiments of the disclosed kits, the kit can comprise one or more cartridges that incorporate one or more substrates. In some embodiments, the one or more cartridges can further comprise one or more pre-loaded solid supports, wherein the individual solid supports within a suspension comprise a plurality of attached stochastic barcodes of the disclosure. In some embodiments, the beads can be pre-distributed into the one or more microwell arrays of the cartridge. In some embodiments, the beads, in the form of suspensions, can be pre-loaded and stored within reagent wells of the cartridge. In some embodiments, the one or more cartridges can further comprise other assay reagents that are pre-loaded and stored within reagent reservoirs of the cartridges.

Disclosed herein are kits for performing spatial analysis of nucleic acids in a sample. The kit can comprise one or more substrates (e.g., array) of the disclosure, either as a free-standing substrate (or chip) comprising one or more arrays. The array can comprise probes of the disclosure. The kit can comprise one or more replicate arrays of the disclosure. The replicate arrays can comprise either gene-specific probes or oligo(dT)/poly(A) probes.

The kit can further comprise reagents, e.g. lysis buffers, rinse buffers, or hybridization buffers, for performing the assay. The kit can further comprise reagents (e.g. enzymes, primers, dNTPs, NTPs, RNase inhibitors, or buffers) for performing nucleic acid extension reactions, for example, reverse transcription reactions and primer extension reactions. The kit can further comprise reagents (e.g. enzymes, universal primers, sequencing primers, target-specific primers, or buffers) for performing amplification reactions to prepare sequencing libraries. The kit can comprise reagents for homopolymer tailing of molecules (e.g., a terminal transferase enzyme, and dNTPs). The kit can comprise reagents for, for example, any enzymatic cleavage of the disclosure (e.g., ExoI nuclease, restriction enzyme).

Kits can generally include instructions for carrying out one or more of the methods described herein. Instructions included in kits can be affixed to packaging material or can be included as a package insert. While the instructions are typically written or printed materials they are not limited to such. Any medium capable of storing such instructions and communicating them to an end user is contemplated by the disclosure. Such media can include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

### Devices

### Flow Cells

The microwell array substrate can be packaged within a flow cell that provides for convenient interfacing with the rest of the fluid handling system and facilitates the exchange of fluids, e.g. cell and solid support suspensions, lysis buffers, rinse buffers, etc., that are delivered to the microwell array and/or emulsion droplet. Design features can include: (i) one or more inlet ports for introducing cell samples, solid support suspensions, or other assay reagents, (ii) one or more microwell array chambers designed to provide for uniform filling and efficient fluid-exchange while minimizing back eddies or dead zones, and (iii) one or more outlet ports for delivery of fluids to a sample collection point or a waste reservoir. The design of the flow cell can include a plurality of microarray chambers that interface with a plurality of microwell arrays such that one or more different cell samples can be processed in parallel. The design of the flow cell can further include features for creating uniform flow velocity profiles, i.e. “plug flow”, across the width of the array chamber to provide for more uniform delivery of cells and beads to the microwells, for example, by using a porous barrier located near the chamber inlet and upstream of the microwell array as a “flow diffuser”, or by dividing each array chamber into several subsections that collectively cover the same total array area, but through which the divided inlet fluid stream flows in parallel. In some embodiments, the flow cell can enclose or incorporate more than one microwell array substrate. In some embodiments, the integrated microwell array/flow cell assembly can constitute a fixed component of the system. In some embodiments, the microwell array/flow cell assembly can be removable from the instrument.

In general, the dimensions of fluid channels and the array chamber(s) in flow cell designs will be optimized to (i) provide uniform delivery of cells and beads to the microwell array, and (ii) to minimize sample and reagent consumption. In some embodiments, the width of fluid channels will be between 50 um and 20 mm. In other embodiments, the width of fluid channels can be at least 50 um, at least 100 um, at least 200 um, at least 300 um, at least 400 um, at least 500 um, at least 750 um, at least 1 mm, at least 2.5 mm, at least 5 mm, at least 10 mm, at least 20 mm, at least 50 mm, at least 100 mm, or at least 150 mm. In yet other embodiments, the width of fluid channels can be at most 150 mm, at most 100 mm, at most 50 mm, at most 20 mm, at most 10 mm, at most 5 mm, at most 2.5 mm, at most 1 mm, at most 750 um, at most 500 um, at most 400 um, at most 300 um, at most 200 um, at most 100 um, or at most 50 um. In one embodiment, the width of fluid channels is about 2 mm. The width of the fluid channels can fall within any range bounded by any of these values (e.g. from about 250 um to about 3 mm).

In some embodiments, the depth of the fluid channels will be between 50 um and 2 mm. In other embodiments, the depth of fluid channels can be at least 50 um, at least 100 um, at least 200 um, at least 300 um, at least 400 um, at least 500 um, at least 750 um, at least 1 mm, at least 1.25 mm, at least 1.5 mm, at least 1.75 mm, or at least 2 mm. In yet other embodiments, the depth of fluid channels can at most 2 mm, at most 1.75 mm, at most 1.5 mm, at most 1.25 mm, at most 1 mm, at most 750 um, at most 500 um, at most 400 um, at most 300 um, at most 200 um, at most 100 um, or at most 50 um. In one embodiment, the depth of the fluid channels is about 1 mm. The depth of the fluid channels can fall within any range bounded by any of these values (e.g. from about 800 um to about 1 mm).

Flow cells can be fabricated using a variety of techniques and materials known to those of skill in the art. In general, the flow cell will be fabricated as a separate part and subsequently either mechanically clamped or permanently bonded to the microwell array substrate. Examples of suitable fabrication techniques include conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining. Once the flow cell part has been fabricated it can be attached to the microwell array substrate mechanically, e.g. by clamping it against the microwell array substrate (with or without the use of a gasket), or it can be bonded directly to the microwell array substrate using any of a variety of techniques (depending on the choice of materials used) known to those of skill in the art, for example, through the use of anodic bonding, thermal bonding, or any of a variety of adhesives or adhesive films, including epoxy-based, acrylic-based, silicone-based, UV curable, polyurethane-based, or cyanoacrylate-based adhesives.

Flow cells can be fabricated using a variety of materials known to those of skill in the art. In general, the choice of material used will depend on the choice of fabrication technique used, and vice versa. Examples of suitable materials include, but are not limited to, silicon, fused-silica, glass, any of a variety of polymers, e.g. polydimethylsiloxane (PDMS; clastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COL), polyethylene terephthalate (PET), epoxy resins, metals (e.g. aluminum, stainless steel, copper, nickel, chromium, and titanium), a non-stick material such as teflon (PTFE), or a combination of these materials.

### Cartridges

In some embodiments of the system, the microwell array, with or without an attached flow cell, can be packaged within a consumable cartridge that interfaces with the instrument system. Design features of cartridges can include (i) one or more inlet ports for creating fluid connections with the instrument or manually introducing cell samples, bead suspensions, or other assay reagents into the cartridge, (ii) one or more bypass channels, i.e. for self-metering of cell samples and bead suspensions, to avoid overfilling or back flow, (iii) one or more integrated microwell array/flow cell assemblies, or one or more chambers within which the microarray substrate(s) are positioned, (iv) integrated miniature pumps or other fluid actuation mechanisms for controlling fluid flow through the device, (v) integrated miniature valves (or other containment mechanisms) for compartmentalizing pre-loaded reagents (for example, bead suspensions) or controlling fluid flow through the device, (vi) one or more vents for providing an escape path for trapped air, (vii) one or more sample and reagent waste reservoirs, (viii) one or more outlet ports for creating fluid connections with the instrument or providing a processed sample collection point, (ix) mechanical interface features for reproducibly positioning the removable, consumable cartridge with respect to the instrument system, and for providing access so that external magnets can be brought into close proximity with the microwell array, (x) integrated temperature control components or a thermal interface for providing good thermal contact with the instrument system, and (xi) optical interface features, e.g. a transparent window, for use in optical interrogation of the microwell array.

The cartridge can be designed to process more than one sample in parallel. The cartridge can further comprise one or more removable sample collection chamber(s) that are suitable for interfacing with stand-alone PCR thermal cyclers or sequencing instruments. The cartridge itself can be suitable for interfacing with stand-alone PCR thermal cyclers or sequencing instruments. The term “cartridge” as used in this disclosure can be meant to include any assembly of parts which contains the sample and beads during performance of the assay.

The cartridge can further comprise components that are designed to create physical or chemical barriers that prevent diffusion of (or increase path lengths and diffusion times for) large molecules in order to minimize cross-contamination between microwells. Examples of such barriers can include, but are not limited to, a pattern of serpentine channels used for delivery of cells and solid supports (e.g., beads) to the microwell array, a retractable platen or deformable membrane that is pressed into contact with the surface of the microwell array substrate during lysis or incubation steps, the use of larger beads, e.g. Sephadex beads as described previously, to block the openings of the microwells, or the release of an immiscible, hydrophobic fluid from a reservoir within the cartridge during lysis or incubation steps, to effectively separate and compartmentalize each microwell in the array.

The dimensions of fluid channels and the array chamber(s) in cartridge designs can be optimized to (i) provide uniform delivery of cells and beads to the microwell array, and (ii) to minimize sample and reagent consumption. The width of fluid channels can be between 50 micrometers and 20 mm. In other embodiments, the width of fluid channels can be at least 50 micrometers, at least 100 micrometers, at least 200 micrometers, at least 300 micrometers, at least 400 micrometers, at least 500 micrometers, at least 750 micrometers, at least 1 mm, at least 2.5 mm, at least 5 mm, at least 10 mm, or at least 20 mm. In yet other embodiments, the width of fluid channels can at most 20 mm, at most 10 mm, at most 5 mm, at most 2.5 mm, at most 1 mm, at most 750 micrometers, at most 500 micrometers, at most 400 micrometers, at most 300 micrometers, at most 200 micrometers, at most 100 micrometers, or at most 50 micrometers. The width of fluid channels can be about 2 mm. The width of the fluid channels can fall within any range bounded by any of these values (e.g. from about 250 um to about 3 mm).

The fluid channels in the cartridge can have a depth. The depth of the fluid channels in cartridge designs can be between 50 micrometers and 2 mm. The depth of fluid channels can be at least 50 micrometers, at least 100 micrometers, at least 200 micrometers, at least 300 micrometers, at least 400 micrometers, at least 500 micrometers, at least 750 micrometers, at least 1 mm, at least 1.25 mm, at least 1.5 mm, at least 1.75 mm, or at least 2 mm. The depth of fluid channels can at most 2 mm, at most 1.75 mm, at most 1.5 mm, at most 1.25 mm, at most 1 mm, at most 750 micrometers, at most 500 micrometers, at most 400 micrometers, at most 300 micrometers, at most 200 micrometers, at most 100 micrometers, or at most 50 micrometers. The depth of the fluid channels can be about 1 mm. The depth of the fluid channels can fall within any range bounded by any of these values (e.g. from about 800 micrometers to about 1 mm).

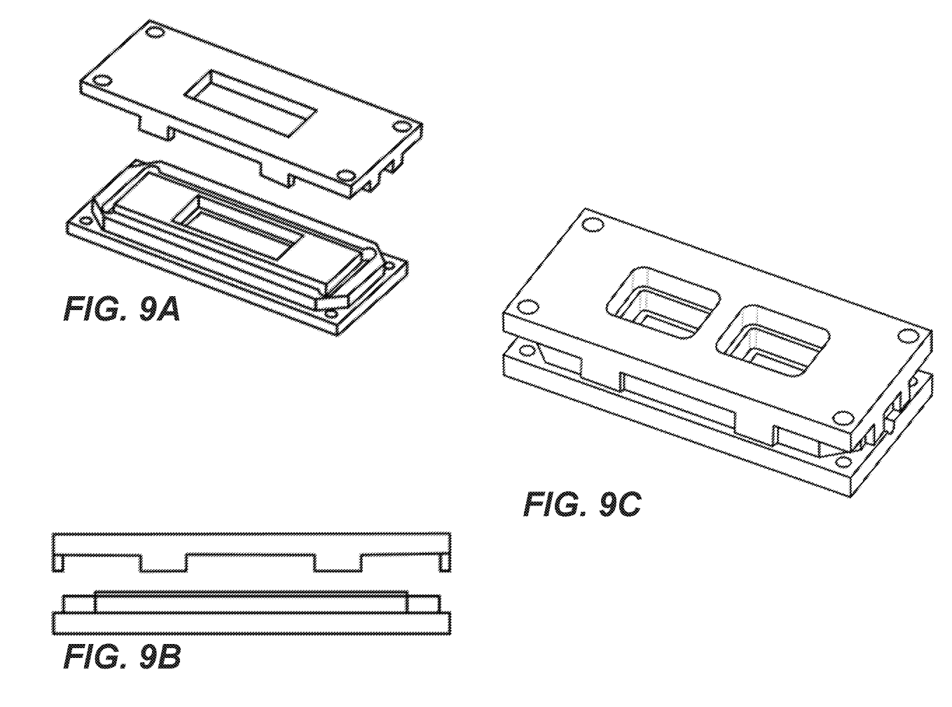

Cartridges can be fabricated using a variety of techniques and materials known to those of skill in the art. In general, the cartridges will be fabricated as a series of separate component parts (FIGS. 9A-C) and subsequently assembled using any of a number of mechanical assemblies or bonding techniques. Examples of suitable fabrication techniques include, but are not limited to, conventional machining. CNC machining, injection molding, thermoforming, and 3D printing. Once the cartridge components have been fabricated they can be mechanically assembled using screws, clips, and the like, or permanently bonded using any of a variety of techniques (depending on the choice of materials used), for example, through the use of thermal bonding/welding or any of a variety of adhesives or adhesive films, including epoxy-based, acrylic-based, silicone-based. UV curable, polyurethane-based, or cyanoacrylate-based adhesives.

Cartridge components can be fabricated using any of a number of suitable materials, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COL), polyethylene terephthalate (PET), epoxy resins, non-stick materials such as teflon (PTFE), metals (e.g. aluminum, stainless steel, copper, nickel, chromium, and titanium), or any combination thereof.

The inlet and outlet features of the cartridge can be designed to provide convenient and leak-proof fluid connections with the instrument, or can serve as open reservoirs for manual pipetting of samples and reagents into or out of the cartridge. Examples of convenient mechanical designs for the inlet and outlet port connectors can include, but are not limited to, threaded connectors. Luer lock connectors, Luer slip or “slip tip” connectors, press fit connectors, and the like. The inlet and outlet ports of the cartridge can further comprise caps, spring-loaded covers or closures, or polymer membranes that can be opened or punctured when the cartridge is positioned in the instrument, and which serve to prevent contamination of internal cartridge surfaces during storage or which prevent fluids from spilling when the cartridge is removed from the instrument. The one or more outlet ports of the cartridge can further comprise a removable sample collection chamber that is suitable for interfacing with stand-alone PCR thermal cyclers or sequencing instruments.

The cartridge can include integrated miniature pumps or other fluid actuation mechanisms for control of fluid flow through the device. Examples of suitable miniature pumps or fluid actuation mechanisms can include, but are not limited to, electromechanically- or pneumatically-actuated miniature syringe or plunger mechanisms, membrane diaphragm pumps actuated pneumatically or by an external piston, pneumatically-actuated reagent pouches or bladders, or electro-osmotic pumps.

The cartridge can include miniature valves for compartmentalizing pre-loaded reagents or controlling fluid flow through the device. Examples of suitable miniature valves can include, but are not limited to, one-shot “valves” fabricated using wax or polymer plugs that can be melted or dissolved, or polymer membranes that can be punctured; pinch valves constructed using a deformable membrane and pneumatic, magnetic, electromagnetic, or electromechanical (solenoid) actuation, one-way valves constructed using deformable membrane flaps, and miniature gate valves.

The cartridge can include vents for providing an escape path for trapped air. Vents can be constructed according to a variety of techniques, for example, using a porous plug of polydimethylsiloxane (PDMS) or other hydrophobic material that allows for capillary wicking of air but blocks penetration by water.

The mechanical interface features of the cartridge can provide for easily removable but highly precise and repeatable positioning of the cartridge relative to the instrument system. Suitable mechanical interface features can include, but are not limited to, alignment pins, alignment guides, mechanical stops, and the like. The mechanical design features can include relief features for bringing external apparatus, e.g. magnets or optical components, into close proximity with the microwell array chamber (FIG. 9B).

The cartridge can also include temperature control components or thermal interface features for mating to external temperature control modules. Examples of suitable temperature control elements can include, but are not limited to, resistive heating elements, miniature infrared-emitting light sources, Peltier heating or cooling devices, heat sinks, thermistors, thermocouples, and the like. Thermal interface features can be fabricated from materials that are good thermal conductors (e.g. copper, gold, silver, etc.) and can comprise one or more flat surfaces capable of making good thermal contact with external heating blocks or cooling blocks.

The cartridge can include optical interface features for use in optical imaging or spectroscopic interrogation of the microwell array. The cartridge can include an optically transparent window, e.g. the microwell substrate itself or the side of the flow cell or microarray chamber that is opposite the microwell array, fabricated from a material that meets the spectral requirements for the imaging or spectroscopic technique used to probe the microwell array. Examples of suitable optical window materials can include, but are not limited to, glass, fused-silica, polymethylmethacrylate (PMMA), polycarbonate (PC), cyclic olefin polymers (COP), or cyclic olefin copolymers (COL).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

### EXAMPLES

Some aspects of the embodiments discussed above are disclosed in further detail in the following examples, which are not in any way intended to limit the scope of the present disclosure.

### Example 1

### Estimating Copy Number of a Target Chromosome

This example describes estimating the copy number of a target chromosome in a sample by partitioning the sample comprising one or more copies of the target chromosome into a plurality of partitioned samples, wherein each of at least 10% of the plurality of partitioned samples comprises one copy of the target chromosome. In this example, the target chromosome is human chromosome 1.

A sample comprising copies of human chromosome 1 is provided. The sample is loaded onto a microfabricated surface with up to 150.000 microwells. Each 30 micron diameter microwell has a volume of approximately 20 picoliters. The concentration of human chromosome 1 is adjusted to 0.01 copy of human chromosome 1 per picoliter by dilution, or one copy of human chromosome 1 per 100 picoliters of the sample. After adjusting the concentration of human chromosome 1 in the sample, 10 picoliters of the sample is loaded onto each of a plurality of microwells. Thus, 1 out of 10 microwells receives a copy of human chromosome 1.

Magnetic beads are loaded onto the microwell array to saturation. The dimension of the bead is chosen such that each microwell may hold only one bead. Each magnetic bead carries approximately one billion stochastic barcodes of oligonucleotides. A stochastic barcode comprises a universal priming site, followed by a chromosome label, a molecular label, and a target binding region. All the stochastic barcodes on each bead have the same chromosome label but contain a diversity of molecular labels. A combinatorial split-pool method can be used to synthesize beads with a diversity of close to one million. The probability of having two copies of the target chromosome being tagged with the same chromosome label is low (on the order of 10⁴) because only 10% of the wells contain one copy of human chromosome 1.

Human chromosome 1 in the microwells is fragmented into 10-kilo base double-stranded nucleotide fragments by sonication. The nucleotide fragments are then denatured by heat to generated single-stranded nucleotide fragments and fast cooled to prevent rehybridization of the single-stranded nucleotide fragments. The human genome contains approximately 3 billion base pairs with approximately 21000 genes. The density of human genes in the human genome is approximately 150000 base pairs per gene. So a gene is fragmented into approximately 15 10-kilo base double-stranded fragments on average. Because the diversity of the molecular labels on a single bead is on the order of 10⁶, the likelihood of two singled-stranded nucleotide fragments of the same gene from the same copy of human chromosome 1 being tagged with the same molecular label is low.

Hybridization buffer is applied onto the surface of the microwell array and diffuses into the microwells. The single-stranded nucleotide fragments hybridize to the target-binding regions on the 3′ end of the stochastic barcodes on the beads. Because the singled-stranded nucleotide fragments are adjacent to the bead, under the high salt conditions of the hybridization buffer and high local concentration of the nucleotide fragments (approximately 26000 10-kilo base single-stranded nucleotide fragments), the singled-stranded nucleotide fragments are captured on the bead.

After hybridization, beads from the microwell array are collected into a tube using a magnet. All reactions in the subsequent experiment steps are carried out in a single tube. DNA synthesis is performed on the beads using conventional protocols. After DNA synthesis, the nucleotide fragments derived from each copy of human chromosome 1 are covalently attached to their corresponding bead, with each tagged on the 5′ end with a chromosome label and a molecular label. Nested multiplex polymerase chain reactions (PCRs) are carried out to amplify genes of interest.

Genes of interest are genes on human chromosome 1. To estimate the copy number of human chromosome 1 in the sample, the kinesin family member 1B (KIF1B) gene can be amplified by nested multiplex PCRs. The copy number of human chromosome 1 can be estimated by the copies of the KIF1B gene. Because the nucleotide fragments from each copy of human chromosome 1 have been copied onto a bead, the beads can be repeatedly amplified and analyzed for a different set of genes. For example, to estimate the copy number of human chromosome 1 in the sample, the brain size determinant (ASPM) gene and the C-reactive protein (CRP) gene can be amplified by nested multiplex PCRs. The copy number of human chromosome 1 in the sample can be estimated by the average number of the ASPM gene and the CRP gene.

Sequencing of the amplicons reveals the chromosome label, the molecular label, and the gene identity. Computational analysis is used to group the reads based on the chromosome label, and collapsed the reads with the same molecular label and gene sequence into a single entry to suppress any amplification bias. The use of the chromosome label and the molecular label enables the measurement of the absolute copy of genes on human chromosome 1, and therefore allow the estimation of the number of human chromosome 1.

### Example 2

### Estimating Copy Numbers of Two Target Chromosomes

This example describes estimating the copy number of two target chromosomes by partitioning a sample comprising one or more copies of each of the two target chromosomes into a plurality of partitioned samples, wherein each of at least 10% of the plurality of partitioned samples comprises one copy of one of the two target chromosomes and each of at least 10% of the plurality of partitioned samples comprises one copy of the other of the two target chromosomes. In this example, the two target chromosomes are human chromosomes 1 and 2.

A sample comprising human chromosome 1 and human chromosome 2 is provided. The sample is loaded onto a microfabricated surface with up to 150.000 microwells. Each 30 micron diameter microwell has a volume of approximately 20 picoliters. The concentration of human chromosome 1 is adjusted to 0.01 copy of human chromosome 1 per picoliter by dilution, or one copy of human chromosome 1 per 100 picoliters of the sample. The dilution also adjusts the concentration of human chromosome 2 to 0.01 copy of human chromosome 2 per picoliter, or one copy of human chromosome 2 per 100 picoliters of the sample. Because a human cell contains one pair of each of chromosomes 1-22 and either a pair of the Y chromosomes or a Y chromosome and an X chromosome, the concentration of chromosomes in the sample is 23 chromosomes per 100 picoliters of the sample. After adjusting the concentration of human chromosome 1 in the sample, 10 picoliters of the sample is loaded onto each of a plurality of microwells. Thus, 1 out of 10 microwells receives a copy of human chromosome 1, and 1 out of 10 microwells receives a copy of human chromosome 2, and 1 out of 100 microwells receives both a copy of human chromosome 1 and a copy of human chromosome 2.

Magnetic beads are loaded onto the microwell array to saturation. The dimension of the bead is chosen such that each microwell may hold only one bead. Each magnetic bead carries approximately one billion stochastic barcodes of oligonucleotides. A stochastic barcode comprises a universal priming site, followed by a chromosome label, a molecular label, and a target binding region. All the stochastic barcodes on each bead have the same chromosome label but contain a diversity of molecular labels. A combinatorial split-pool method can be used to synthesize beads with a diversity of close to one million. The probability of having two copies of the target chromosome being tagged with the same chromosome label is low (on the order of 10⁴) because only 10% of the wells contain one copy of human chromosomes 1 or 2.

The copies of human chromosomes 1 and 2 in the microwells are fragmented into 10-kilo base double-stranded nucleotide fragments by sonication. The nucleotide fragments are then denatured by heat to generated single-stranded nucleotide fragments and fast cooled to prevent rehybridization of the single-stranded nucleotide fragments. The human genome contains approximately 3 billion base pairs with approximately 21000 genes. The density of human genes in the human genome is approximately 150000 base pairs per gene. So a gene is fragmented into approximately 15 10-kilo base (kb) double-stranded fragments on average. Because the diversity of the molecular labels on a single bead is on the order of 10⁶, the likelihood of two singled-stranded nucleotide fragments of the same gene from the same copy of human chromosomes 1 or 2 being tagged with the same molecular label is low.

Hybridization buffer is applied onto the surface of the microwell array and diffuses into the microwells. The single-stranded nucleotide fragments hybridize to the target-binding regions on the 3′ end of the stochastic barcodes on the beads. Because the singled-stranded nucleotide fragments are adjacent to the bead, under the high salt conditions of the hybridization buffer and high local concentration of the nucleotide fragments, the singled-stranded nucleotide fragments are captured on the bead. A microwell with one copy of human chromosome 1 and no human chromosome 2 has approximately 26000 10-kilo base (kb) single-stranded nucleotide fragments of chromosome 1. A microwell with one copy of each of human chromosomes 1 and 2 and no other human chromosome has approximately 52000 10-kilo base (kb) single-stranded nucleotide fragments of chromosomes.

After hybridization, beads from the microwell array are collected into a tube using a magnet. All reactions in the subsequent experiment steps are carried out in a single tube. DNA synthesis is performed on the beads using conventional protocols. After DNA synthesis, the nucleotide fragments derived from each copy of human chromosomes 1 and 2 are covalently attached to their corresponding bead, with each tagged on the 5′ end with a chromosome label and a molecular label. Nested multiplex polymerase chain reactions (PCRs) are carried out to amplify genes of interest.

Genes of interest are genes on human chromosomes 1 and 2. To estimate the copy number of human chromosome 1 in the sample, the kinesin family member 1B (KIF1B) gene can be amplified by nested multiplex PCRs. The copy number of human chromosome 1 can be estimated by the copies of the KIF1B gene. To estimate the copy number of human chromosome 2 in the sample, the otoferlin (OTOF) gene can be amplified by nested multiplex PCRs. The copy number of human chromosome 2 can be estimated by the copies of the OTOF gene.

Because the nucleotide fragments from each copy of human chromosome 1 have been copied onto a bead, the beads can be repeatedly amplified and analyzed for a different set of genes. For example, to estimate the copy number of human chromosome 1 in the sample, the brain size determinant (ASPM) gene and the C-reactive protein (CRP) gene can be amplified by nested multiplex PCRs. The copy number of human chromosome 1 can be estimated by the average number of the ASPM gene and the CRP gene. To estimate the copy number of human chromosome 2 in the sample, the ATP-binding cassette, sub-family A (ABC1), member 12 (ABCA12) gene and the bone morphogenetic protein receptor, type II (serine/threonine kinase) (BMPR2) gene can be amplified by nested multiplex PCRs. The copy number of human chromosome 2 can be estimated by the average number of the ABCA12 gene and the BMPR2 gene.

Sequencing of the amplicons reveals the chromosome label, the molecular label, and the gene identity. Computational analysis is used to group the reads based on the chromosome label, and collapsed the reads with the same molecular label and gene sequence into a single entry to suppress any amplification bias. The use of the chromosome label and the molecular label enables the measurement of the absolute copy of genes on human chromosomes 1 and 2, and therefore allow the estimation of the copy numbers of human chromosomes 1 and 2.

### Example 3

### Haplotype Phasing

This example describes haplotype phasing of two or more gene targets on a target chromosome, for example human chromosome 1, in a sample by partitioning the sample comprising one or more copies of human chromosome 1 into a plurality of partitioned samples, wherein each of at least 10% of the plurality of partitioned samples comprises one copy of human chromosome 1.

A sample comprising one or more copies of human chromosome 1 is provided. The sample is loaded onto a microfabricated surface with up to 150,000 microwells. Each 30 micron diameter microwell has a volume of approximately 20 picoliters. The concentration of human chromosome 1 is adjusted to 0.01 copy of human chromosome 1 per picoliter by dilution, or one copy of human chromosome 1 per 100 picoliters of the sample. After adjusting the concentration of human chromosome 1 in the sample, 10 picoliters of the sample is loaded onto each of a plurality of microwells. Thus, 1 out of 10 microwells receives a copy of human chromosome 1.

Magnetic beads are loaded onto the microwell array to saturation. The dimension of the bead is chosen such that each microwell may hold only one bead. Each magnetic bead carries approximately one billion stochastic barcodes of oligonucleotides. A stochastic barcode comprises a universal priming site, followed by a chromosome label, a molecular label, and a target binding region. All the stochastic barcodes on each bead have the same chromosome label but contain a diversity of molecular labels. A combinatorial split-pool method can be used to synthesize beads with a diversity of close to one million. The probability of having two copies of the target chromosome being tagged with the same chromosome label is low (on the order of 10⁻⁴) because only 10% of the wells contain one copy of human chromosome 1.

Human chromosome 1 in the microwells are fragmented into 10-kilo base double-stranded nucleotide fragments by sonication. The nucleotide fragments are then denatured by heat to generated single-stranded nucleotide fragments and fast cooled to prevent rehybridization of the single-stranded nucleotide fragments. The human genome contains approximately 3 billion base pairs with approximately 21000 genes. The density of human genes in the human genome is approximately 150000 base pairs per gene. So a gene is fragmented into approximately 15 10-kilo base (kb) double-stranded fragments on average. Because the diversity of the molecular labels on a single bead is on the order of 10⁶, the likelihood of two singled-stranded nucleotide fragments of the same gene from the same copy of human chromosome 1 being tagged with the same molecular label is low.

Hybridization buffer is applied onto the surface of the microwell array and diffuses into the microwells. The single-stranded nucleotide fragments hybridize to the target-binding regions on the 3′ end of the stochastic barcodes on the beads. Because the singled-stranded nucleotide fragments are adjacent to the bead, under the high salt conditions of the hybridization buffer and high local concentration of the nucleotide fragments (approximately 26000 10-kilo base (kb) single-stranded nucleotide fragments), the singled-stranded nucleotide fragments are captured on the bead.

After hybridization, beads from the microwell array are collected into a tube using a magnet. All reactions in the subsequent experiment steps are carried out in a single tube. DNA synthesis is performed on the beads using conventional protocols. After DNA synthesis, the nucleotide fragments derived from each copy of human chromosome 1 are covalently attached to their corresponding bead, with each tagged on the 5′ end with a chromosome label and a molecular label. Nested multiplex polymerase chain reactions (PCRs) are carried out to amplify genes of interest.

Genes of interest are the two or more gene targets on human chromosome 1. To determine the haplotype phasing of the two or more gene targets on human chromosome 1, for example the brain size determinant (ASPM) gene and the C-reactive protein (CRP), nucleotide fragments of these two gene can be amplified by nested multiplex PCRs. Because the nucleotide fragments from each copy of human chromosome 1 have been copied onto a bead, the beads can be repeatedly amplified and analyzed for a different set of genes. For example, to determine the haplotype phasing of the UDP-galactose-4-epimerase (GALE) gene and the mitofusin 2 (MFN2) gene, fragments of these two gene can be amplified by nested PCRs.

Sequencing of the amplicons reveals the chromosome label, the molecular label, and the gene identity. Computational analysis is used to group the reads based on the chromosome label, and collapsed the reads with the same molecular label and gene sequence into a single entry to suppress any amplification bias. The use of the chromosome label and the molecular label enables the determination of gene variants on human chromosome 1, and therefore allow haplotype phasing of the two or more gene targets on human chromosome 1.

### Example 4

### Determining Aneuploidy of One or More Cells

This example describes determining ancuploidy of one or more cells using stochastic barcoding.

A sample comprising a target chromosome, for example human chromosomes 1, from one or more cells is provided. The sample is loaded onto a microfabricated surface with up to 150,000 microwells. Each 30 micron diameter microwell has a volume of approximately 20 picoliters. The concentration of human chromosome 1 is adjusted to 0.01 copy of human chromosome 1 per picoliter by dilution, or one copy of human chromosome 1 per 100 picoliters of the sample. After adjusting the concentration of human chromosome 1 in the sample, 10 picoliters of the sample is loaded onto each of a plurality of microwells. Thus, 1 out of 10 microwells receives a copy of human chromosome 1.

Magnetic beads are loaded onto the microwell array to saturation. The dimension of the bead is chosen such that each microwell may hold only one bead. Each magnetic bead carries approximately one billion stochastic barcodes of oligonucleotides. A stochastic barcode comprises a universal priming site, followed by a chromosome label, a molecular label, and a target binding region. All the stochastic barcodes on each bead have the same chromosome label but contain a diversity of molecular labels. A combinatorial split-pool method can be used to synthesize beads with a diversity of close to one million. The probability of having two copies of the target chromosome being tagged with the same chromosome label is low (on the order of 10⁻⁴) because only 10% of the wells contain one copy of human chromosome 1.

The copies of human chromosome 1 in the microwells are fragmented into 10-kilo base double-stranded nucleotide fragments by sonication. The nucleotide fragments are then denatured by heat to generated single-stranded nucleotide fragments and fast cooled to prevent rehybridization of the single-stranded nucleotide fragments. The human genome contains approximately 3 billion base pairs with approximately 21000 genes. The density of human genes in the human genome is approximately 150000 base pairs per gene. So a gene is fragmented into approximately 15 10-kilo base (kb) double-stranded fragments on average. Because the diversity of the molecular labels on a single bead is on the order of 10⁶, the likelihood of two singled-stranded nucleotide fragments of the same gene from the same copy of human chromosome 1 being tagged with the same molecular label is low.

Hybridization buffer is applied onto the surface of the microwell array and diffuses into the microwells. The single-stranded nucleotide fragments hybridize to the target-binding regions on the 3′ end of the stochastic barcodes on the beads. Because the singled-stranded nucleotide fragments are adjacent to the bead, under the high salt conditions of the hybridization buffer and high local concentration of the nucleotide fragments (approximately 26000 10-kilo base (kb) single-stranded nucleotide fragments), the singled-stranded nucleotide fragments are captured on the bead.

After hybridization, beads from the microwell array are collected into a tube using a magnet. All reactions in the subsequent experiment steps are carried out in a single tube. DNA synthesis is performed on the beads using conventional protocols. After DNA synthesis, the nucleotide fragments derived from each copy of human chromosome 1 are covalently attached to their corresponding bead, with each tagged on the 5′ end with a chromosome label and a molecular label. Nested multiplex polymerase chain reactions (PCRs) are carried out to amplify genes of interest.

Genes of interest are genes on human chromosome 1. To estimate the copy number of human chromosome 1 in the sample, the kinesin family member 1B (KIF1B) gene can be amplified by nested multiplex PCRs. The copy number of human chromosome 1 can be estimated by the copies of the KIF1B gene. Based on the copy number of the cells and the number of human chromosome in the sample, aneuploidy of the cells for human chromosome 1 is determined. The copy number of human chromosomes 1-22 and the human X and Y chromosomes are determined, and the aneuploidies of the cells for each human chromosome are determined.

Sequencing of the amplicons reveals the chromosome label, the molecular label, and the gene identity. Computational analysis is used to group the reads based on the chromosome label, and collapsed the reads with the same molecular label and gene sequence into a single entry to suppress any amplification bias. The use of the chromosome label and the molecular label enables the measurement of the absolute copy of genes on human chromosome 1, and therefore allow the estimation of copy number of human chromosome 1 and the determination of aneuploidy of the one or more cells in the sample.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least.” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B. and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A. B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for estimating copy number of a target chromosome in a sample, comprising:
providing a sample comprising one or more copies of a first target chromosome;
partitioning the sample into a plurality of partitioned samples, wherein each of at least 10% of the plurality of partitioned samples comprises one copy of the first target chromosome;
stochastically barcoding the one or more copies of the first target chromosome in the plurality of partitioned samples using a first plurality of stochastic barcodes, wherein each of the first plurality of stochastic barcodes comprises a first chromosome label and a first molecular label; and
estimating the copy number of the first target chromosome in the sample using the first chromosome label and the second molecular label.

2. The method of claim 1, wherein partitioning the sample comprises adjusting the volume of the sample to alter the concentration of the first target chromosome in the sample.

3. The method of claim 1, wherein partitioning the sample comprises adjusting the volume of the sample partitioned into each of the plurality of partitioned samples.

4. The method of claim 1, wherein each of at least 25% of the plurality of partitioned samples comprises one copy of the first target chromosome.

5.-7. (canceled)

8. The method of claim 1, wherein each of the plurality of partitioned samples is introduced to a well of a plurality of wells of a substrate.

9. The method of claim 1, wherein each of the plurality of partitioned samples is a droplet in an emulsion.

10. (canceled)

11. The method of claim 1, wherein stochastically barcoding the one or more copies of the first target chromosome in the plurality of partitioned samples comprises generating one or more copies of a stochastically barcoded first target chromosome.

12. The method of claim 11, wherein stochastically barcoding the one or more copies of the first target chromosome comprises generating an indexed library of the stochastically barcoded first target chromosome.

13. The method of claim 1, wherein stochastically barcoding the one or more copies of the first target chromosome comprises fragmenting the one or more copies of the first target chromosome to generate fragments of the first target chromosome.

14. The method of claim 13, wherein the fragments of the first target chromosome are at least 10 kilo bases (kb), 100 kb, or 1000 kb in length.

15.-16. (canceled)

17. The method of claim 13, wherein the stochastically barcoded first target chromosome comprises stochastically barcoded fragments of the first target chromosome.

18. The method of claim 1, wherein the first plurality of the stochastic barcodes is associated with a solid support.

19.-20. (canceled)

21. The method of claim 18, wherein the first chromosome labels of the first plurality of stochastic barcodes on the solid support are the same.

22. The method of claim 1, wherein the first chromosome label is about 5-20 nucleotides long.

23.-25. (canceled)

26. The method of claim 17, wherein estimating the copy number of the first target chromosome in the sample comprises determining sequences of at least some of the stochastically barcoded fragments of the first target chromosome in the indexed library.

27. (canceled)

28. The method of claim 1, wherein the one or more copies of the first target chromosome are inside or not inside one or more cells.

29.-32. (canceled)

33. The method of claim 1,
wherein the sample comprises one or more copies of a second target chromosome, and
wherein each of at least 10% of the plurality of partitioned samples comprises one copy of the second target chromosome,
the method further comprising:
stochastically barcoding one or more copies of the second target chromosome in the plurality of partitioned samples using a second plurality of stochastic barcodes, wherein each of the second plurality of stochastic barcodes comprises a second chromosome label and a second molecular label, and wherein the first chromosome labels of the first plurality of stochastic barcodes and the second chromosome labels of the second plurality of stochastic barcodes differ by at least one nucleotide; and
estimating the copy number of the second target chromosome in the sample using the second chromosome label and the second molecular label.

34. The method of claim 1,
wherein the sample comprises one or more copies of each of n target chromosomes, wherein n is an integer greater than one, and
wherein, for each of the n target chromosomes, each of at least 10% of the plurality of partitioned samples comprises one copy of the n^{th} target chromosome,
the method further comprising:
for each of the n target chromosomes in the plurality of partitioned samples, stochastically barcoding the one or more copies of the n^{h} target chromosome using a n^{th} plurality of stochastic barcodes,
wherein each of the n^{th} plurality of stochastic barcodes comprises a n^{th} chromosome label and a n^{th} molecular label, and wherein the first chromosome labels of the first plurality of stochastic barcodes and the n^{th} chromosome labels of the n^{th} plurality of stochastic barcodes differ by at least one nucleotide; and
estimating the copy number of each of the plurality of n^{th} target chromosomes in the sample using the n^{th} chromosome label and the n^{th} molecular label.

35. (canceled)

36. A method for haplotype phasing two or more gene targets on a target chromosome in a sample, comprising:
providing a sample comprising one or more copies of a target chromosome, wherein the target chromosome comprises two or more gene targets;
partitioning the sample into a plurality of partitioned samples, wherein each of at least 10% of the plurality of partitioned samples comprises one copy of the target chromosome;
stochastically barcoding the one or more copies of the target chromosome in the plurality of partitioned samples using a plurality of stochastic barcodes, wherein each of the plurality of stochastic barcodes comprises a chromosome label and a molecular label; and
determining the haplotype phasing of the two or more gene targets on the target chromosome in the sample using the chromosome label and the molecular label.

37.-51. (canceled)

52. The method of claim 36, wherein at least two of the two or more gene targets are separated from one another on the target chromosome by at least 10 kb, at least 100 kb, or at least 1000 kb.

53.-54. (canceled)

55. A method for determining aneuploidy of one or more cells, comprising:
providing a sample comprising chromosomes from one or more cells;
partitioning the sample into a plurality of partitioned samples, wherein each of at least 10% of the plurality of partitioned samples comprises one copy of a first target chromosome;
stochastically barcoding the one or more copies of the first target chromosome in the plurality of partitioned samples using a first plurality of stochastic barcodes, wherein each of the first plurality of stochastic barcodes comprises a first chromosome label and a first molecular label; and
determining the aneuploidy of the one or more cells in the sample, wherein determining the aneuploidy of the one or more cells in the sample comprises determining the number of a first gene target on the first target chromosome using the first chromosome label and the first molecular label.

56.-67. (canceled)

68. The method of claim 55, wherein the aneuploidy is a trisomy.

69. The method of claim 68, wherein the trisomy is an autosomal trisomy.

70.-71. (canceled)

72. A method for sequencing a first target chromosome in a sample, comprising:
providing a sample comprising one or more copies of a first target chromosome;
partitioning the sample into a plurality of partitioned samples, wherein each of at least 10% of the plurality of partitioned samples comprises one copy of the first target chromosome;
stochastically barcoding the one or more copies of the first target chromosome in the plurality of partitioned samples using a first plurality of stochastic barcodes, wherein each of the first plurality of stochastic barcodes comprises a first chromosome label and a first molecular label; and
obtaining sequence information of the first target chromosome using the first chromosome label and the first molecular label.

73.-91. (canceled)
